SEQUENCE LISTING (22 sequences)

SEQ ID NO: 1 - DNA, 4074 bp, Homo sapiens
tacctccggctctcccgggtgacgacgggtagaaaagcaggaggagcggagaaaggagagggcggggtagggatgcagctgtgctgcattctgggaagggcgttggtccgtcgctcgcgcagcctcctgggagttgtagtcgcgatcctgaggtaacggataagtttataccatggatagcacaaaggagaagtgtgacagttacaaagatgatcttctgcttaggatgggacttaatgataataaagcaggaatggaaggattagataaagagaaaattaacaaaattataatggaagccacgaaggggtccagattttatggaaatgagctcaagaaagaaaagcaagtcaaccaacgaattgaaaatatgatgcaacaaaaagctcaaatcaccagccaacagctaagaaaagcacaattacaggttgacagatttgcaatggaattagaacaaagccgaaatttgagcaataccatagtgcacattgacatggatgctttctatgcagctgtagaaatgagggacaatccagaattgaaggataaacccattgctgtaggatcaatgagtatgctgtctacttcaaattaccatgcaaggagatttggtgttcgtgcagccatgccaggatttattgctaagaggctgtgcccacaacttataatagtgccccccaactttgacaaataccgagctgtgagtaaagaggttaaggaaatacttgctgattatgatcccaattttatggccatgagtcttgatgaagcctacttgaatataacaaagcacttagaagaaagacaaaattggcctgaggataaaagaaggtatttcatcaaaatgggaagctctgtagaaaatgataatccaggaaaggaagttaataaactgagtgagcatgaacgatccatctctccactactttttgaagagagtccttctgatgtgcagcccccaggagatcctttccaagtgaactttgaagaacaaaacaatcctcaaatactccaaaactcagttgtttttggaacatcagcccaggaagtggtaaaggaaattcgtttcagaattgagcagaaaacaacactgacagccagtgcaggcattgccccaaatacaatgttagcaaaagtatgcagtgataagaataaaccaaatggacaataccaaattcttcccaatagacaagctgtgatggacttcatcaaggatttacccattagaaaggtttctggaataggaaaagttacagagaaaatgttaaaggcccttggaattattacatgtacagaactttaccaacagagggcattgctttctctccttttctctgaaacatcttggcattatttccttcatatctccttgggtctaggttcaacacacctgacgagggatggagagaggaaaagtatgagcgttgagaggacattcagtgagataaataaagcggaagagcaatacagcctatgtcaagaactttgcagtgagcttgctcaggatctacagaaagaaagacttaagggtagaactgttaccattaagttgaagaatgtgaattttgaagtaaaaactcgtgcatctacagtttcatctgttgtttctactgcagaagaaatatttgccattgctaaggaattgctaaaaacagaaattgatgctgattttccacatcccttgagattaaggcttatgggtgttcggatatctagttttcccaatgaagaggacaggaaacaccaacaaaggagcattattggctttttacaggctggaaaccaagccctgtcagccactgagtgtacattagagaaaactgacaaagataagtttgtaaaacctctagaaatgtctcataagaagagtttctttgataaaaaacgatcagaaaggaaatggagtcaccaagatacatttaaatgtgaagccgtgaataaacaaagtttccagacatcacaaccattccaagttttaaagaagaagatgaatgagaatttggaaatatcagagaattcagatgactgtcagatacttacctgtcctgtttgctttagggctcaagggtgcatcagtctggaagccttgaataaacatgtagatgaatgtcttgatggaccttcaatcagtgaaaactttaaaatgttctcgtgttcacatgtttctgctaccaaagttaacaagaaagaaaatgttcctgcttcttcactttgtgagaagcaagattatgaagcccatccaaaaattaaagaaatatcttcagtagattgtatagctttagtagatactatagataactcatctaaagcagaaagcatagatgctttaagtaataagcatagcaaggaagaatgttctagtctcccaagcaagtcttttaatattgaacactgtcatcagaattcttcttctactgtttcattggaaaacgaagatgttggatcatttagacaagaataccgccagccttacttatgtgaagtgaaaacaggccaagctctagtttgtcctgtttgtaacgtagaacaaaagacttcagatctaaccctgttcaatgtgcatgtggatgtttgcttaaataaaagttttatccaagaattaagaaaggataaatttaacccagttaatcaacccaaagaaagctccagaagtactggtagctcaagtggagtacagaaggctgtaacaagaacaaaaaggccaggattgatgacaaagtactcaacatcaaagaaaataaaaccaaacaatcccaaacatacccttgatatattttttaagtaaacattgaacattttatcattaatttttaattgaaactagttattttataatcaatgaatttgttctttctgattttaagtttgcagatttatttagtgaaggcaagtgcaataatccttcctcagatgatgtttgcttttctaagatacatatactgattctgtgtatcttttttataaccatgagaattttacttccattatacatcaattggaaatcaatcctgttaagagataattcttaaaagggaaattaggaatgggataagaaggtgatttttttattatttttatactgaatataaaaacatttgtaagggctctcaaagattcacacatgcctatattatcataagaatttttcagcacttaactactttgttggcattgatcctagtgtctttaaatacttcatgagcattcataattaaaatttatcttaagttctatgaagagtattaatgtaactagcataagtggtttcttcaggaaaataaatatcacagtattatctgtgttaaaatggtttttgcctaaaatataatttttaatttggcttttcttatttaaaattccattatcttatgaataagcacttgaatcagtttttaaaatatttagtctaagatgattcaaagtagttttattttaatacaggacttttaaatggcagtatttcatttcttgtcaattatgttggtactttccacaaatctataaagaaggataaattgtaccatcattttattataatcctcaagagaaaatgtgtaattcaaaagattaatgtgtattaaaacacattatgtatcttagttacatttctatcagtacttttattaatatttgtgaaagaagacagcttaatagtagttagcttaagtagtttctccaagtacttttgtgctatcaatgagttcttctcaaaaaataattagttaggccaggcacaatggctcacacctgtaatgccagccctttgggaggccgaatgggcagatcacttgaggtcaggagtttgagaccagcctcgccaacatggtgagaccctgtctctactaaaacgataaaaaaaaaaaaaaaattagccaggcttggtggcacacgcctgtaatcccagctactcagatggctgaggcaggagaactacttgaacctgggaggtcaaagctgcagtcagccaagatcttgccactgtactccagcctgaagagcgagactctgtctcaataatataataatagttattatttaattgcaacatgaagttggaagccattttctgttact

SEQ ID NO: 2 - protein, 870 aa, Homo sapiens
MDSTKEKCDSYKDDLLLRMGLNDNKAGMEGLDKEKINKIIMEATKGSRFYGNELKKEKQVNQRIENMMQQKAQITSQQLRKAQLQVDRFAMELEQSRNLSNTIVHIDMDAFYAAVEMRDNPELKDKPIAVGSMSMLSTSNYHARRFGVRAAMPGFIAKRLCPQLIIVPPNFDKYRAVSKEVKEILADYDPNFMAMSLDEAYLNITKHLEERQNWPEDKRRYFIKMGSSVENDNPGKEVNKLSEHERSISPLLFEESPSDVQPPGDPFQVNFEEQNNPQILQNSVVFGTSAQEVVKEIRFRIEQKTTLTASAGIAPNTMLAKVCSDKNKPNGQYQILPNRQAVMDFIKDLPIRKVSGIGKVTEKMLKALGIITCTELYQQRALLSLLFSETSWHYFLHISLGLGSTHLTRDGERKSMSVERTFSEINKAEEQYSLCQELCSELAQDLQKERLKGRTVTIKLKNVNFEVKTRASTVSSVVSTAEEIFAIAKELLKTEIDADFPHPLRLRLMGVRISSFPNEEDRKHQQRSIIGFLQAGNQALSATECTLEKTDKDKFVKPLEMSHKKSFFDKKRSERKWSHQDTFKCEAVNKQSFQTSQPFQVLKKKMNENLEISENSDDCQILTCPVCFRAQGCISLEALNKHVDECLDGPSISENFKMFSCSHVSATKVNKKENVPASSLCEKQDYEAHPKIKEISSVDCIALVDTIDNSSKAESIDALSNKHSKEECSSLPSKSFNIEHCHQNSSSTVSLENEDVGSFRQEYRQPYLCEVKTGQALVCPVCNVEQKTSDLTLFNVHVDVCLNKSFIQELRKDKFNPVNQPKESSRSTGSSSGVQKAVTRTKRPGLMTKYSTSKKIKPNNPKHTLDIFFK

SEQ ID NO: 3 - DNA, 4263 bp, Mus musculus
gtgtcctgggcgcgcctaaaggctggttgcctaggggaaccttctgaaggcaagtgggcttcttttgagagttgcgtgcccctttcggtccagcctggcttccgattctgccttgcgtgtttgtgacgagccagcgagccgggacgtgagaaccctcagatattaagaaattaccctgtttgcatcatggataacacaaaggaaaaggacaacttcaaagacgacctcctgctccgcatgggactaaacgataacaaagcaggcatggaagggttggataaggagaaaattaacaaaattatcatggaagccacaaaggggtccagattttatggaaatgagctcaagaaggaaaagcaagtcaatcaacggattgaaaatatgatgcaacaaaaagctcaaattaccagccagcaactaaggaaagctcaattacaggttgacaaatttgcaatggagttagaacggaaccggaatttgaacaataccatagttcatgttgacatggacgctttctatgcagctgtggaaatgagggacaacccggaactgaaggataaacccattgctgtaggatccatgagcatgttggctacttcgaattaccatgcaaggaggtttggtgtccgtgcagccatgccaggatttattgctaagaggctctgcccacaacttattatagtgcccccaaactttgacaaatatagagctgtgagtaaggaggttaaggagatacttgctgaatatgatcccaattttatggccatgagtctggacgaagcctacttgaatataacacagcacttgcaggaaaggcaagattggcctgaggacaaaagaagatacttcatcaaaatgggaaactacttaaaaatcgacacacccagacaggaagctaacgagctgactgagtatgagcggtccatctccccgctgctttttgaagatagtcctcctgatttgcaaccccaaggaagtcctttccaactgaactctgaagaacaaaacaatcctcaaatagcccaaaattcagttgtttttggaacatcagctgaggaagtggtaaaggaaattcgcttcagaattgaacaaaaaacaacgctgacagccagcgcaggcatcgcccccaatacaatgttagcaaaagtgtgcagtgataagaataagccaaacggacagtaccagatccttcccagcaggagcgcggtgatggacttcatcaaggacctgcctattagaaaggtttctgggataggaaaagttacagagaaaatgttaatggctctcgggattgttacttgcacagaactctaccaacagagagcgttgctgtctctccttttctctgaaacctcttggcattattttcttcacatcgcgctgggtctaggttcaacagacctggcaagggatggagaaaggaaaagcatgagtgttgaaaggacattcagtgagataagtaagacagaggaacagtacagcctgtgccaagaactgtgcgctgagctcgcccacgacctccagaaggaaggacttaagggaagaaccgtcaccattaagctgaagaacgtgaattttgaagtaaaaactcgtgcatctaccgttccggccgccatttctactgcagaggaaatatttgccattgccaaggagctgctaaggacagaagttaatgtgggttctccacaccccctgcggttaagactgatgggtgtccgaatgtctactttttccagtgaagatgacaggaaacaccaacaaaggagcatcattggtttcttacaagctggaaaccaagctttgtcatctactggggatagtctagacaaaactgacaaaactgagcttgcaaagcccttagaaatgtctcataagaagagtttctttgataaaaagcgatcagaaagaatctccaactgtcaagacacatccagatgtaaaactgcgggtcagcaagctttacagatcttggaaccatcccaagcattaaagaagctgagcgagagttttgaaacatcagagaattcaaatgactgtcagacatttatatgtccagtttgctttagggagcaagaaggtgtcagtctggaagcctttaatgaacatgtagatgagtgtcttgatggaccgtcaaccagtgagaactcaaaaatatcctgttactcacatgcttcctctgcagacattggtcagaaggaagatgtacacccctctattccactgtgtgagaaacgggggcatgaaaatggagagatcactttagtagatggtgtagatctaacagggacggaagacagatcattgaaagcagcaaggatggacactctagagaataatcgcagcaaagaggaatgtcctgatattccagacaagtcttgtcctatatcactggaaaatgaaaccatcagtacattaagtaggcaagactctgtccagccttgtacagatgaggtagtaacaggacgagctctagtgtgtcctgtttgtaacctagaacaagagacttctgatcttaccctcttcaacatacatgtggatatttgcttaaataaaggtattatccaagaactgagaaatagtgaaggtaattcagttaaacaacccaaagaaagctcgagaagtactgacagacttcagaaggcttcaggaaggaccaaaaggccaggaacgaagacaaagagctcaactttgaagaaaacaaagccccgagatcccagacacacccttgatggattttttaaatgaactttgaacattttatcaacgtttatcattgaaattattattttcatagtcaatatatttattcttcctcattttaaatgtattcctttaaggaagacaagtgcaataatgcctcccctacgtgacctttttaagaatgtagactgaatattaatttatttcatttatgttttccttaatagccatgagaatttcattcccagtatatatatatatatcttagttggaaatcagtcgtgttagagacagtgtaagaagtgaggtcagaaagtgatgcgttattttataatggatataaaatatttccaaggactctgagccacatggagcaacctcaccatcagctcctccatgcttagtcaccatggtgccattgatgcttccgtacctacctgtgtgcatctctgtgtcctgtgagcagatgcagttagaatcggccatgatgtccatgaagaacattagcttaacataaagagtgtgggcatggctgttcccttcaggaaggtgatgcacagtattatctgtgttcagaggacctgtccaaacctgtgatttgtattcttacttatcttacatataagcacttggttcacataaatatttagtccataattcaaaatcttttttttaactcagtactttaaatggcagtatttaatttcttaacatttatatttgactacaaatctgcagtaaacaaggtaagttgtgtagttgcctggttttcattcccctctttaggagaaaatagacacctcagaagatgtgccctgaggagtcactgcacttaccgtagctgcttctctgtcgtctctgctaattgttgtgggaggaagatttgcagcaattcagaggttgctgcagatgtagtaccacccacaagttcttaccaaaaagcccttcctgctactactttaagaactgaattctgctgggttgtggtggtgcacgcctttaatcccagcactgggaggcagaggcagaggcaggtggatctctgaatttgaggacaacctggtctacagagtgagttccagaaagaacagcctggaccacacagagaaaccctacttcaaaacacaaaaaaggaaaaaaaaaaccctgaatattttttatagtcttgcctttgcttattttaatgcatttattatagacagagagatatttgatggtccttagaaccgaccttgtatggcacagtcttaacctgacttttaattgctagactgcttaaaaaaatgggctaaaagctcgctgacacaccagacccttacctgatattatacaccacaacagttccaactctgtggtcctttggttattggttgtgatttacttatgtaaactatttataaaattaaattcaatgaaactgatttaatgtattgaaaaatagatattactgtattattttcttccatctgaagtttatttttgttgttttcaacattaagtaataaattattttcatgtagatgctaaaaaaaaaaaaaaaaaaaaaaaaaaaaaaaaaaaaaa

SEQ ID NO: 4 - protein, 852 aa, Mus musculus
MDNTKEKDNFKDDLLLRMGLNDNKAGMEGLDKEKINKIIMEATKGSRFYGNELKKEKQVNQRIENMMQQKAQITSQQLRKAQLQVDKFAMELERNRNLNNTIVHVDMDAFYAAVEMRDNPELKDKPIAVGSMSMLATSNYHARRFGVRAAMPGFIAKRLCPQLIIVPPNFDKYRAVSKEVKEILAEYDPNFMAMSLDEAYLNITQHLQERQDWPEDKRRYFIKMGNYLKIDTPRQEANELTEYERSISPLLFEDSPPDLQPQGSPFQLNSEEQNNPQIAQNSVVFGTSAEEVVKEIRFRIEQKTTLTASAGIAPNTMLAKVCSDKNKPNGQYQILPSRSAVMDFIKDLPIRKVSGIGKVTEKMLMALGIVTCTELYQQRALLSLLFSETSWHYFLHIALGLGSTDLARDGERKSMSVERTFSEISKTEEQYSLCQELCAELAHDLQKEGLKGRTVTIKLKNVNFEVKTRASTVPAAISTAEEIFAIAKELLRTEVNVGSPHPLRLRLMGVRMSTFSSEDDRKHQQRSIIGFLQAGNQALSSTGDSLDKTDKTELAKPLEMSHKKSFFDKKRSERISNCQDTSRCKTAGQQALQILEPSQALKKLSESFETSENSNDCQTFICPVCFREQEGVSLEAFNEHVDECLDGPSTSENSKISCYSHASSADIGQKEDVHPSIPLCEKRGHENGEITLVDGVDLTGTEDRSLKAARMDTLENNRSKEECPDIPDKSCPISLENETISTLSRQDSVQPCTDEVVTGRALVCPVCNLEQETSDLTLFNIHVDICLNKGIIQELRNSEGNSVKQPKESSRSTDRLQKASGRTKRPGTKTKSSTLKKTKPRDPRHTLDGFFK

SEQ ID NO: 5 - protein, 7 aa, Mus musculus
YFAAVEM

SEQ ID NO: 6 - protein, 9 aa, Mus musculus
NKPNGQYFV

SEQ ID NO: 7 - DNA, 27 bp, Mus musculus
  modified_base (16)..(21): N = A, C, G or t/U
cgaattctayttygcngcgtngaratg

SEQ ID NO: 8 - DNA, 29 bp, Mus musculus
  modified_base (12): W = A/T
cgggatccacrwaytgccrttggyttrtt

SEQ ID NO: 9 - DNA, 24 bp, Homo sapiens
tggatagcacaaaggagaagtgtg

SEQ ID NO: 10 - DNA, 24 bp, Homo sapiens
aatctggaccccttcgtggcttcc

SEQ ID NO: 11 - DNA, 34 bp, Homo sapiens
gtggatccgccatggatagcacaaaggagaagtg

SEQ ID NO: 12 - DNA, 43 bp, Homo sapiens
catacccttgatatattttttaagtagtcgaccgcggatccat

SEQ ID NO: 13 - DNA, 34 bp, Homo sapiens; identical to SEQ ID NO: 11

SEQ ID NO: 14 - DNA, 61 bp, Homo sapiens
atggatccgcggtcgactaatggtggtgatgatggtgcttaaaaaatatatcaagggtatg

SEQ ID NO: 15 - DNA, 63 bp, Homo sapiens
atggatccgcggtcgactaatggtggtgatgatggtgagatctacccataagccttaatctca

SEQ ID NO: 16 - DNA, 24 bp, Homo sapiens
gagctcccaaagctttggatgcat

SEQ ID NO: 17 - DNA, 26 bp, Homo sapiens
ccatgagtcttgctgcagcctacttg

SEQ ID NO: 18 - protein, 15 aa, Mus musculus
CNYLKIDTPRQEANE

SEQ ID NO: 19 - DNA, 55 bp, Artificial Sequence
  Description of Artificial Sequence  Synthetic Primer
attccagactgtcaataacacggtgggaccagtcgatcctgggctgcaggaattc

SEQ ID NO: 20 - DNA, 30 bp, Artificial Sequence
  Description of Artificial Sequence  Synthetic Primer
gaattcctgcagcccaggatcgactggtcc

SEQ ID NO: 21 - DNA, 27 bp, Mus musculus
aggccatggataacacaaaggaaaagg

SEQ ID NO: 22 - DNA, 33 bp, Mus musculus
acggtcgacacgttgataaaatgttcaaagttc

(19) United States
(12) Patent Application Publication
(10) Pub. No.: US 2003/0017573 A1
(43) Pub. Date: Jan. 23, 2003

(54) Polymerase kappa compositions and methods thereof

(75) Inventors: Errol C. Friedberg, Dallas, TX (US); Valerie Gerlach, Branford, CT (US); William J. Feaver, Branford, CT (US)

Correspondence Address: Gina N. Shishima, Fulbright & Jaworski L.L.P., Suite 2400, 600 Congress Avenue, Austin, TX, 78701, US

(73) Assignee: Board of Regents, The University of Texas system

(21) Appl. No.: 09/971,101

(22) Filed: Oct. 4, 2001

Related U.S. Application Data
(60) Provisional application No. 60/238,289, filed on Oct. 4, 2000.

(51) Int. Cl. C12N 9/64; C07H 21/04; C12P 21/02; C12N 5/06

(52) U.S. Cl. 435/226; 435/69.1; 435/325; 435/320.1; 536/23.2

(57) ABSTRACT

The present invention concerns compositions and methods involving mammalian polymerase kappa, an enzyme with limited fidelity and moderate processivity. Methods of modulating polymerase kappa activity, such as inhibiting or reducing its activity, as a means of effecting a cancer treatment or preventative agent are provided, both by itself and in combination with other anti-cancer therapies. Also described are methods of screening involving assaying for polymerase kappa activity or expression, in addition to methods of screening for modulators of polymerase kappa to identify anti-cancer compounds.

This application claims the priority of U.S. Provisional Application Ser. No. 60/238,289, filed Oct. 4, 2000, the entire disclosure of which is specifically incorporated herein by reference. The government may own rights in the present invention pursuant to grant numbers CA 75733 and CA69029 from the National Cancer Institute.

### BACKGROUND OF THE INVENTION

1. Field of the Invention

The present invention relates generally to the fields of biochemistry and cancer diagnosis and therapy. More particularly, it concerns polymerase kappa (pol κ), the POL κ gene encoding it, and its relevance to cancer and other conditions or diseases involving DNA mutations and repair pathways.

2. Description of Related Art

a. Cancer

Second only to heart disease, cancer, is the leading cause of death in the United States, striking one in two men and one in three women (Landis, 1998). Lung carcinoma is the most predominant form of cancer leading to death in men in the United States for several decades. Moreover, mortality rates among women from lung cancer in the United States recently surpassed breast cancer mortality rates (Landis, 1998). Almost one third of all deaths caused by cancer can be attributed to cancer of the lung.

The development of cancer is understood as the culmination of complex, multistep biological processes, occurring through the accumulation of genetic alterations. Many if not all of these alterations involve specific cellular growth-controlling genes that are mutated. These genes typically fall into two categories: proto-oncogenes and tumor suppressor genes. Mutations in genes of both classes generally confer a growth advantage on the cell containing the altered genetic material.

The function of tumor suppressor genes, as opposed to proto-oncogenes, is to antagonize cellular proliferation. When a tumor suppressor gene is inactivated, for example by point mutation or deletion, the cell's regulatory machinery for controlling growth is upset. Studies from several laboratories have shown that the neoplastic tendencies of such mutated cells can be suppressed by the addition of a nonmutated (wild-type) version of the tumor suppressor gene that expresses its gene product (Levine, 1995).

The gene products of proto-oncogenes, as alluded to above, typically are involved in pathways of normal cell growth or differentiation. Many of the participants of these pathways, when genetically mutated, contribute to the promotion of tumor development and the genes encoding them are consequently termed “oncogenes.” The polypeptides encoded by proto-oncogenes include transcriptions factors (e.g., c-fos, c-jun, c-myc), growth factor receptors (e.g., c-fms, c-erbB, c-kit), growth factors (e.g., c-sis, iznt-2) and cell cycle proteins (e.g., PRAD1). Mutations in one or more proto-oncogenes—that is, the presence of one or more oncogenes—has been shown to be associated with specific cancers. Unlike tumor suppressors genes involved in cancer, oncogenes express a protein product that possesses activity. Thus, the treatment of cancer may involve inactivating, inhibiting, or reducing the activity of one or more oncogene products.

Currently, there are few effective options for the treatment of many common cancer types. The course of treatment for a given individual depends on the diagnosis, the stage to which the disease has developed and factors such as age, sex and general health of the patient. The most conventional options of cancer treatment are surgery, radiation therapy and chemotherapy. These therapies each are accompanied with varying side effects and they have varying degrees of efficacy. Furthermore, gene therapy is an emerging field in biomedical research with a focus on the treatment of disease by the introduction of therapeutic recombinant nucleic acids into somatic cells of patients. Various clinical trials using gene therapies have been initiated and include the treatment of various cancers, AIDS, cystic fibrosis, adenosine deaminase deficiency, cardiovascular disease, Gaucher's disease, rheumatoid arthritis, and others. However, there is a continued need for effective cancer therapies.

b. DNA Polymerases

In E. coli, mutagenesis associated with exposure to DNA-damaging agents requires a specialized system, the SOS system, which processes the damage in an error-prone fashion, resulting in mutations (Friedberg et al. 1995). Recent in vitro studies with purified reconstituted systems have shown that E. coli UmuC protein, in conjunction with UmuD′ protein (both of which are encoded by SOS-regulated genes (Friedberg et al., 1995)), single-strand binding protein and activated RecA protein, can facilitate error-prone bypass of DNA lesions by DNA polymerase III holoenzyme (Reuven et al., 1998, Tang et al., 1998). The dinB gene of E. coli (sometimes referred to as dinP (Ohmori et al., 1995)) also is regulated by the SOS system, and is required for untargeted (spontaneous) mutations in phage λ when infected cells are exposed to ultraviolet (UV) radiation (Brotcorne-Lannoye et al., 1986). Additionally, overexpression of the cloned dinB gene in unirradiated E. coli cells carrying plasmids dramatically increases the mutational burden in the plasmid DNA (Kim et al., 1997). E. coli DinB protein recently has been purified and shown to have a specialized DNA polymerase activity (Wagner et al., 1999).

E. coli DinB protein is homologous to an uncharacterized protein from C. elegans (F22B7.6), the S. cerevisiae Rev1 protein, and E. coli UmuC protein (Ohmori et al., 1995). Like UmuC protein, Rev1 is involved in DNA damage-induced mutagenesis in yeast (Larimer et al., 1989). Rev1 protein has been shown to possess a novel DNA polymerase (deoxycytidyl transferase) activity which efficiently inserts dCMP residues opposite sites of base loss in a template/primer-dependent reaction (Nelson et al., 1996). More recently, the yeast Rad30 protein, which is also homologous to UmuC and DinB (McDonald et al., 1997; Roush et al., 1998), has been shown to be a DNA polymerase (DNA pol η) which accurately replicates thymine dimers in template DNA (Johnson et al., 1999). A human homolog of Rad30 has properties very similar to that of yeast DNA pol η (Masutani et al., 1999), and patients from the variant group of the cancer-prone hereditary disease xeroderma pigmentosum (XP-V) have been shown to carry mutations in this homolog of RAD30 (Johnson et al., 1999b; Masutani et al., 1999). Collectively, these observations suggest that members of the UmuC/DinB superfamily all are replication-bypass DNA polymerases. However, these may differ in their fidelity and/or affinity for various types of damaged DNA.

The cloning and characterization of mouse and human homologs of the E. coli dinB gene are described herein. In some references, the mouse and human genes have been referred to as Dinb1 and DINB1, respectively, and the gene products as DinB1 or pol theta (see Johnson et al., 2000). Moreover, the TRF4 gene product has been referred to as pol kappa (Wang et al., 2000), however, the present invention does not concern TRF4. With respect to the present invention, the homolog of the E. coli dinB gene is referred to as POLK, for the human gene (Genbank accession #AF163570 for the cDNA sequence), and Polk, for the mouse gene (Genbank accession #AF163571 for the cDNA sequence). The gene product, termed polymerase kappa or polymerase κ (pol κ) (Genbank accession #AAF02540 for human and #AAF02541 for mouse polypeptides), has limited fidelity and moderate processivity. The compositions and methods of the present invention are based on its role in hyperproliferative diseases or conditions, particularly cancer. As there is a need for therapies to treat cancer, as well as other mutation-based diseases, it is the object of the present invention to provide methods and compositions that involve reducing or inhibiting pol κ function as well as methods of identifying and using modulators of pol κ.

### SUMMARY OF THE INVENTION

The present invention takes advantage of the isolation and characterization of human and mouse homologs of the E. coli dinB gene, whose product has been characterized as a DNA polymerase. Therefore, the present invention is directed at therapeutic and diagnostic methods and compositions involving POLK (human) and Polk (mouse) nucleic acids and Pol κ polypeptide compositions (human and mouse), as well as modulators that affect POLK, Polk, and Pol κ. Any of the nucleic acid- and proteinaceous compound-containing compositions disclosed herein may be practiced with respect to other compositions and methods of the invention.

Compositions of the present invention include isolated and purified polynucleotides that include a nucleic acid sequence that encodes a mammalian pol κ polypeptide. A mammalian pol κ polypeptide is a polypeptide that is identified as a homolog of E coli DinB and is found in a mammalian organism, such as a human, monkey, gorilla, mouse, cow, sheep, lamb, and rat. In particular embodiments of the present invention, a human or murine polypeptide are specifically contemplated.

In some aspects of the invention, compositions involve a polynucleotide that includes a nucleic acid sequence encoding a segment of contiguous amino acids from SEQ ID NO:2 (human amino acid sequence, corresponding to Genbank accession no. AAF02540) or SEQ ID NO:4 (murine amino acid sequence, corresponding to Genbank accession no. AAF02540). Segments may constitute all or part of a pol κ polypeptide. Nucleic acid and amino acid sequences may be of varying lengths. Thus, the present invention covers polynucleotides including all or part of mammalian pol κ coding regions, such as SEQ ID NO:1 (human pol κ cDNA sequence corresponding to Genbank accession no. AF163570) and SEQ ID NO:3 (murine pol κ cDNA sequence, corresponding to Genbank accession no. AF163571); polynucleotides that include a nucleic acid sequence encoding all or part of a mammalian pol κ polypeptide or peptide, such as a contiguous amino acid sequence from SEQ ID NO:2 and SEQ ID NO:4; polypeptides and peptides including all or part of a mammalian pol κ polypeptide, such as a contiguous amino acid sequence of SEQ ID NO:2 and SEQ ID NO:4; and, polypeptides and peptides encoded for by a contiguous nucleic acid sequence of SEQ ID NO:1 or SEQ ID NO:3.

Other compositions of the invention include expression vectors comprising a nucleic acid sequence encoding a mammalian pol κ polypeptide or peptide, such as a murine or human pol κ polypeptide. As discussed above and herein, expression vectors of the present invention may include contiguous nucleic acid sequences from SEQ ID NO:1 or SEQ ID NO:3 or that encode all or part of SEQ ID NO:2 or SEQ ID NO:4. Expression vectors may viral vectors or non-viral vectors. In some embodiments, a promoter is included in the vector, and the promoter may be operably linked to a heterologous sequence, such as a nucleic acid sequence encoding all or part of a mammalian pol κ polypeptide. The promoter may be constitutive, inducible, tissue-specific.

The present invention also encompasses methods of preparing a mammalian pol κ polypeptide, peptide, or polynucleotide. Such methods may be accomplished by (a) transfecting a host cell with a polynucleotide comprising a nucleic acid sequence encoding a pol κ polypeptide and b) maintaining the transformed host cell under biological conditions sufficient for expression of the pol κ polypeptide in the host cell. As previously discussed, it is specifically contemplated that any of the nucleic acid compositions disclosed herein may be employed in the practice of this method.

In some embodiments of the present invention, methods of treating a pre-cancer or cancer cell are included. These methods involve providing to a pre-cancer or cancer cell an effective amount of a pol κ modulator, wherein the modulator reduces pol κ activity in the cell.

The invention encompasses treatments of pre-cancer or cancer in which the following types of cells are targeted: bladder, blood, bone, bone marrow, brain, breast, colon, esophagus, gastrointestine, gums, head, kidney, liver, lung, nasopharynx, neck, ovary, prostate, skin, stomach, testis, tongue, and uterus cell. It is further contemplated that a cell contacted in the method of the present invention is a non-small cell lung carcinoma cell, such as a squamous carcinoma cell, an adenocarcinoma cell, or a large-undifferentiated carcinoma cell, or is a small cell lung carcinoma cell. The present invention also includes the treatment of pre-cancer or cancer in a subject who exhibits a solid tumor.

It is contemplated that a pol κ modulator reduces pol κ activity by reducing the ability of pol κ to bind to or polymerize a nucleic acid molecule, decreases the amount of pol κ in the cell, decreases expression of pol κ, decreases transcription of pol κ, decreases translation of pol κ, specifically binds pol κ, or otherwise exerts an effect of pol κ activity. In some embodiments, a pol κ modulator is a polypeptide, such as an antibody, agonist, or antagonist. In other embodiments, a pol κ modulator is provided to the cell by an expression cassette comprising a nucleic acid segment encoding the modulator. For instance, the modulator of pol κ may be a nucleic acid molecule containing a promoter operably linked to a nucleic acid segment encoding at least 30 contiguous nucleotides of SEQ ID NO:1 or SEQ ID NO:3. The nucleic acid segment may also be positioned in reverse orientation under the control of a promoter that directs expression of an antisense product.

It is contemplated that any of the treatment methods of the invention may be performed in vitro, in vivo, or ex vivo. Thus, in some embodiments, cells that are administered or provided a composition may be located in an organism.

Treatment methods of the invention may also include additional anti-cancer treatments, in addition to compositions of the present invention. The additional anti-cancer treatment may be surgery, gene therapy, chemotherapy, radiotherapy, or immunotherapy. Treatment with compositions of the invention, or additional anti-cancer treatments may given to the subject simultaneously, or one may be given before the other. It is contemplated that there may be a lag between different therapies. In some embodiments, one or more of the treatments is repeated at least once, if not multiple times.

The invention also includes methods of treating a pre-cancer or cancer cell by contacting the cell with an effective amount of an expression vector that includes a polynucleotide encoding a pol κ polypeptide under the transcriptional control of a promoter, wherein the cancer cell is conferred a therapeutic benefit. The term “therapeutic benefit” used throughout this application refers to anything that promotes or enhances the well-being of the subject with respect to the medical treatment of his condition, which includes treatment of pre-cancer, cancer, and hyperproliferative diseases. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition. It is contemplated that any of the methods described with respect to the treatment of cancer may also be employed for the prevention of cancer.

In some embodiments of the present invention, methods of reducing DNA mutagenesis in a cell are described. Such methods may be effected by administering a pol κ modulator in an amount effective to reduce DNA mutagenesis in the cell; other steps may also be included in the practice of these methods. Other methods of the invention include ways of increasing DNA mutagenesis in a cell by providing to the cell an expression vector comprising a polynucleotide encoding a pol κ polypeptide under the transcriptional control of a promoter, wherein expression of the pol κ polypeptide is at a level effective to increase mutagenesis in the cell. Any of the compositions of the present invention may be used to implement these methods.

Other methods involve treating a patient with pre-cancer or cancer by administering to the patient an amount of a pol κ modulator effective to reduce pol κ activity, thereby conferring a therapeutic benefit on the subject.

In further aspects of the invention, there are methods of identifying a modulator of a pol κ polypeptide comprising: (a) contacting the pol κ polypeptide with a candidate substance; and (b) assaying whether the candidate substance modulates the pol κ polypeptide. In still further aspects, methods involve comparing the activity of the pol κ polypeptide in the presence and absence of the candidate substance or determining whether the candidate substance specifically interacts with the polκ polypeptide.

Methods of diagnosing cancer in a subject are also part of the invention. These methods involve obtaining a sample from the subject and evaluating pol κ in the sample. Pol κ may be evaluated by assaying the level of pol κ activity, assaying the amount of pol κ polypeptide, for example, with an antibody that specifically binds pol κ, or by evaluating a genomic DNA or cDNA sequence encoding pol κ from the subject. Such methods would be well known to those of ordinary skill in the art.

Another method of the present invention involves treating a subject with a trinucleotide repeat disease or a subject susceptible to a trinucleotide repeat disease by administering to the subject an effective amount of an expression vector that includes a polynucleotide encoding a pol κ polypeptide under the transcriptional control of a promoter, such that a pol κ polypeptide is expressed in the subject. Alternatively, a modulator of pol κ expression may be administered to the subject in an amount effective to increase pol κ expression. Trinucleotide repeat diseases that may be treated include Fragile X syndrome, Fragile XE syndrome, Friedreich ataxia, myotonic dystrophy, spinocerebellar ataxia (types 1, 2, 3, 6, 7, 8, and 12), spinobulbar muscular atrophy, Huntington's disease, and Haw-River syndrome. (See Cummings et al., 2000 for review). Any of the regimens, compositions, and methods relevant to the treatment and diagnosis of cancer may be used with respect to the treatment of a trinucleotide repeat disease. Combination therapy with a trinucleotide repeat disease therapeutic agent and pol κ gene therapy are specifically contemplated by the present invention.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Spectra of errors by pol κ₁₋₅₆₀ and full-length pol κ.

FIG. 1A: Shows distribution of single-base substitutions. The 407-nucleotide template DNA sequence is shown as four lines of template sequence, with nucleotide +1 as the first transcribed nucleotide of the LacZ α-complementation gene in M13mp2 DNA. DNA synthesis begins with incorporation opposite template nucleotide +191 (arrow in the bottom line of sequence), and the last single-stranded template nucleotide in the gap is at position −216 (arrow in top line of sequence). The termination codon for the C-terminal end of the LacI gene in M13mp2 is underlined (nucleotides −87, −86 and −85). Also underlined is the sequence of the palindromic Lac operator that can form a hairpin structure in the template strand. Single-base substitutions generated by pol κ₁₋₅₆₀ are shown above the template sequence and those generated by full-length pol κ are shown below the sequence. FIG. 1B: Shows distribution of deletions and additions. Errors generated by pol κ₁₋₅₆₀ are shown above the template sequence and those generated by full-length pol κ are shown below the sequence. Single-base deletions are depicted by open triangles and two-base deletions are depicted by adjacent open red triangles. Single-base additions are shown with a letter to indicate the added base, and a slanted line indicating where that base was added. When deletions or additions occur within repetitive sequences, the actual base that is deleted or added is not known.

FIG. 2. Expression of pol kappa protein in murine deletion mutants for exon 6 of dinB.

FIG. 3. GST/pol kappa is able to bypass a thymine glycol adduct in vivo.

FIG. 4. GST/pol kappa preferentially incorporates adenine opposite thymine glycol.

FIG. 5. Multiple dinB transcripts are found mouse testis.

FIG. 6. p53-dependent induction of dinB gene expression occurs in response to genotoxic stress.

### DESCRIPTION OF ILLUSTRATIVE EMBODIMENTS

The present invention is based on the isolation of mouse and human dinB/DINB genes, characterization of the polymerase polypeptides encoded by those genes, and observation of a role for these polymerases in therapeutic settings, particularly with respect to the treatment of cancer.

I. Polymerase Kappa

A. Escherichia coli dinB Gene

Historically, the identification of these novel DNA polymerases resulted from efforts to understand molecular mechanisms that relieve arrested (by multiple forms of base damage) semi-conservative DNA replication catalyzed by high-fidelity, high-processivity replicative polymerases. As observed initially in E. coli and subsequently in lower and higher eukaryotes, the relief of arrested semi-conservative DNA replication associated with base damage is frequently accompanied by mutations at or near sites of such damage (Friedberg et al., 1995). It was initially postulated that the resumption of normal DNA replication is effected by relaxation of the high fidelity of the normal replicative polymerases, thereby facilitating error-prone replicative bypass (translesion synthesis (TLS)) (Friedberg et al., 1995). The pioneering studies of Evelyn Witkin, Miroslav Radman, Bryn Bridges and others established that in E. coli TLS is intimately associated with the SOS phenomenon whereby base damage to DNA (and other perturbations of DNA function) results in the coordinated up-regulation of a large regulon of genes (Friedberg et al., 1995). These observations prompted the late Hatch Echols and his colleagues to search for proteins whose expression is regulated by the SOS system, and which might facilitate the participation of high fidelity replicative DNA polymerases in error-prone TLS in vitro.

These and other studies led to the identification of a role of the SOS-regulated umuC and umuD genes in TLS and DNA damage-induced mutagenesis in E. coli (Friedberg et al., 1995). Subsequent studies showed that the UmuC and UmuD proteins indeed participate in TLS as a specialized UmuD′₂C complex (Tang et al., 1998; Reuven et al., 1999; Tang et al., 1999). More recently it has been demonstrated that the purified UmuD′₂C complex is itself a novel DNA polymerase (Tang et al., 1999; Reuven et al., 1999; Tang et al., 2000) which is capable of effecting TLS of sites of template base damage on simple DNA primer-templates in vitro. The UmuD′₂C complex is now designated DNA polymerase V of E. coli (Tang et al., 1999).

The E. coli dinB gene encodes a protein homologous to UmuC (Ohmori et al., 1995) and is required for SOS-dependent untargeted mutagenesis of phage λ (Brotcorne-Lannoye et al., 1986). In addition, overexpression of the dinB gene has been shown to result in increased mutagenesis, in particular, −1 frameshift mutations (Kim et al., 1997). The dinB gene was subsequently shown to encode DNA polymerase IV, a strictly distributive enzyme which lacks detectable 3′Ø5′ proofreading exonuclease activity (Wagner et al., 1999). Since many, but not all, of the spontaneous mutations associated with SOS-dependent induction of dinB are −1 frameshifts, initial examination of the properties of purified DNA pol IV focused on its ability to support DNA synthesis at model replication forks which might mimic template slippage (Wagner et al., 1999). It recently has been shown that, in contrast to DNA pol V, DNA pol IV is unable to bypass UV radiation-induced lesions or abasic sites in vitro, suggesting that DNA pol IV does not play a major role (if any) in TLS of such lesions in vivo (Tang et al., 2000).

These observations have marked the emergence of a revised model for TLS of damaged or modified DNA. Rather than requiring protein-dependent modification of the normal replicative machinery, the revised model suggests that, when normal semi-conservative DNA synthesis is arrested, the replicative polymerase is replaced by one of a set of novel DNA polymerases whose primary function is to support the incorporation of a limited number of nucleotides opposite the offending lesion(s) in the template strand. Different DNA polymerases may be required for the bypass of different types of DNA damage and/or structures at replication forks. This model provides a compelling explanation for the molecular mechanism of TLS and identifies a general mechanism for both DNA damage-induced and spontaneous mutagenesis in E. coli.

A general prediction of this model is that different novel DNA polymerases are specific for TLS of different classes of base damage or other perturbations of DNA structure, which result in arrested DNA replication. The in vitro properties of several novel eukaryotic DNA polymerases fulfill this prediction. Thus, a novel enzyme with deoxycytidyl transferase activity encoded by the REV1 gene of the yeast S. cerevisiae has been shown to preferentially insert C opposite sites of base loss in template DNA (Nelson et al., 1996b), and a DNA polymerase encoded by the yeast RAD30 gene and human RAD30A gene, called DNA pol η, has been shown to replicate past cis-syn thymine-thymine dimers, correctly inserting adenine opposite the damage (Johnson et al., 1999b). The properties of DNA pol η suggest an anti-mutagenesis function, since whether by default or as an intrinsic property of DNA pol η to “read” thymine residues in a dimerized conformation, the incorporation of adenine allows replicative bypass in an error-free manner. Consistent with this anti-mutagenic role of DNA pol η humans defective in the human ortholog of RAD30 (XPV or POLH gene) suffer from the skin cancer-prone hereditary disease xeroderma pigmentosum (XP) (Johnson et al., 1999c; Matsutani et al., 1999a; Matsutani et al., 1999b).

B. Prokaryotic and Eukaryotic Homologs

The mammalian DinB1 proteins are members of the growing UmuC/DinB superfamily of DNA polymerases. The phylogenetic relationships between members of this superfamily have been examined. Examination of this tree reveals four distinct branches with multiple members that are convincingly supported by the bootstrap test, as well as a single member (SsoDINB) possibly representing a fifth branch. Two of the four confirmed branches (RAD30 and REV1) are exclusively eukaryotic, one (UmuC) is exclusively bacterial, and one (DinB) includes both eukaryotic and bacterial (E. coli DinB) proteins. Interestingly, the DinB branch lacks obvious orthologs in S. cerevisiae and D. melanogaster.

All members of the UmuC/DinB superfamily contain conserved sequences, which include an N-terminal nucleotidyl transferase domain, two tandem helix-hairpin-helix (HhH) modules implicated in DNA-binding, and a weakly conserved C-terminal domain. There is no significant overall sequence similarity between the DinB-family nucleotidyl transferase domain and previously identified DNA polymerases (or any other enzymes). However, the DinB/UmuC superfamily contains two highly conserved motifs which center at an invariant Asp-Glu (DE) doublet and a highly conserved AspXAsp (DXD) signature present in most family members. This pattern of conserved negatively charged residues is present in the catalytic centers of all previously characterized families of polymerases and nucleotidyl transferases, in which the acidic residues are believed to coordinate divalent cations directly involved in catalysis.

A similar role for these residues can be predicted for the UmuC/DinB family nucleotidyl transferases. This prediction is supported by the observation that both residues of the invariant DE doublet are essential for the DNA polymerase activities of E. coli pol IV (Wagner et al., 1999) and S. cerevisiae pol η (Johnson et al., 1999). The HhH motif is a common nucleic acid-binding module found in a variety of proteins involved in DNA replication, recombination and repair, and the duplicated HhH module in the UmuC/DinB polymerases is predicted to mediate DNA-binding. The specific function of the C-terminal conserved domain remains to be elucidated.

The eukaryotic branches of this superfamily possess unique, evolutionarily conserved domain architectures that should be regarded as shared derived characters supporting the tree topology. Specifically, proteins within the Rev1 subgroup also possess an N-terminal BRCT domain, suggesting a role in cell cycle checkpoint functions. The Rad30 subgroup is characterized by a C-terminal C2H2 Zn-finger which is absent in the pol ι proteins and is partially disripted in the S. cerevisiae pol η protein. Finally, the eukaryotic members of the DinB subgroup contain a C-terminal C2HC Zn-cluster module, which is duplicated in the mammalian DinB1 proteins. This distinct type of Zn-cluster is found in combination with other enzymatic and binding domains in two known DNA repair proteins, S. cerevisiae Snm1 and Rad18. Since Rad18 is a DNA-binding protein which contains only two identifiable domains, namely a RING finger and the C2HC Zn-cluster, and given the known role of the RING domain in specific protein-protein interactions, it can be predicted that the Zn-cluster binds DNA. Hence, the eukaryotic DinB homologs likely contain two DNA-binding domains, the HhH motif and the Zn-cluster.

The early stages of the evolutionary history of the UmuC/DinB superfamily of TLS-associated polymerases are uncertain because of horizontal gene transfer and lineage-specific gene loss. The importance of these modes of evolution is supported both by the patchy distribution of these polymerases in bacteria and archaea (with only one archaeal member identified so far in the crenarchaeon Sulfolobus solfataricus (Kuleava et al., 1996)), and by the location of the umuC genes on plasmids and the uvrX gene in a bacteriophage. It appears that at least one gene coding for this type of polymerase was present at the base of the eukaryotic crown group, with an early duplication resulting in the emergence of the Rev1 and Rad30 (Polη/ι) families. A subsequent duplication, probably at an early stage of metazoan evolution, resulted in the divergence of pol η and pol ι.

The phylogenetic affinity of the eukaryotic DinB proteins with their ortholog from E. coli and the presence, in these proteins, of putative mitochondrial import sequences suggests a mitochondrial origin as well as a potential function in mitochondrial DNA metabolism for these polymerases. Gene transfer from mitochondria to the nucleus should have been accompanied by the fusion of the Zn-cluster-coding sequence with the polymerase gene. The mammalian DinB1 proteins (now referred to as polymerase κ) also contain a good match to a bipartite nuclear localization signal at their C-terminus.

C. Proteinaceous Compositions

In certain embodiments, the present invention concerns novel compositions comprising at least one proteinaceous molecule, such as pol κ or a modulator of pol κ, such as an antibody against pol κ. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3- Amino adipic acid | Hyl | Hydroxylysine |
| Bala | β-alanine, β-Amino-propionic acid | AHyl | allo-Hydroxylysine |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least one antibody, for example, an antibody against pol κ. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow et al., 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

1. Functional Aspects

When the present application refers to the function or activity of pol κ or it is meant that the molecule in question has the ability to polymerize DNA or generally to promote the introduction of a mutation or mutations in genomic DNA. Other phenotypes that may be considered to be associated with the normal pol κ gene product are template-directed DNA polymerization with limited fidelity, enhancing of spontaneous frameshift and base substitution mutagenesis, generating DNA products that are one or two nucleotides shorter than full-length, generating DNA products with moderate—as opposed to high—processivity, generating DNA products with high termination probabilities, the ability to promote transformation of a cell from a normally regulated state of proliferation to a malignant state, i.e., one associated with any sort of abnormal growth regulation, or to promote the transformation of a cell from an abnormal state to a highly malignant state, e.g., to promote metastasis or invasive tumor growth, and an effect on angiogenesis, adhesion, migration, cell-to-cell signaling, cell growth, cell proliferation, density-dependent growth, anchorage-dependent growth and others. Determination of which molecules possess this activity may be achieved using assays familiar to those of skill in the art, For example, transfer of genes encoding products that inhibit or modulate pol κ, or variants thereof, into cells that have a functional pol κ product, and hence exhibit mutagenized DNA or impaired growth control, will identify, by virtue of decreased mutation rate, those molecules having pol κ modulator or inhibitor function. An endogenous pol κ polypeptide refers to the polypeptide encoded by the cell's genomic DNA.

Fidelity in the context of polymerase activity refers to the overall accuracy of polymerization with respect to the template molecule. “Limited fidelity” or “reduced fidelity” means that the overall average base substitution error rate is higher than that for polymerases that replicate the nuclear genome; thus, “limited fidelity” defines a polymerase that has an error rate greater than 1×10⁻⁵. Error rate may be the error rate for single-base substitution, single-base deletions, or single-base additions. Processivity in the context of polymerase activity refers to the ability to synthesize a polynucleotide stretch before disengaging from the template molecule. It is defined as the number of nucleotides polymerized per cycle of polymerase association/dissociation. High processivity is considered to be the ability to synthesize at least 1×10³ nucleotides/binding event. High processivity enzymes include T7 DNA polymerase with thioredoxin and replicative DNA polymerase δ and ε in the presence of auxiliary processivity-enhancing proteins, e.g., PCNA. DNA pol ε on its own can synthesize 1×10²/binding event. Low processivity enzymes such as DNA polymerase β or κ synthesize less than 10 nucleotides/binding event. Thus, moderate processivity is understood to be above 10 nucleotides/binding event, but below 1×10⁴ nucleotides/binding event. Pol κ is similar to Klenow with respect to processivity. Termination probability refers to the likelihood that a polymerase will dissociate from the template at any given point. While variation in termination probability for a given polymerase has been observed, for example a rate of 30%-60% for pol η, the termination probability to pol κ has a range of 0%-50% at any particular spot, depending on the sequence.

On the other hand, when the present invention refers to the function or activity of a “pol κ modulator,” one of ordinary skill in the art would further understand that this includes, for example, the ability to specifically or competitively bind pol κ or an ability to reduce or inhibit its activity. Thus, it is specifically contemplated that a pol κ modulator may be a molecule that affects pol κ expression, such as by binding a pol κ-encoding transcript. Determination of which molecules are suitable modulators of pol κ may be achieved using assays familiar to those of skill in the art—some of which are disclosed herein—and may include, for example, the use of native and/or recombinant pol κ.

2. Variants of Pol κ and Pol κ Modulators

Amino acid sequence variants of the polypeptides of the present invention can be substitutional, insertional or deletion variants. Deletion variants lack one or more residues of the native protein that are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of a pol κ polypeptide or a modulator of a pol κ provided the biological activity of the protein is maintained.

The term “functionally equivalent codon” is used herein to refer to codons that encode the same amino acid, such as the six codons for arginine or serine, and also refers to codons that encode biologically equivalent amino acids (see Table 2, below).

TABLE 2
Codon Table

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAG AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 2 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of pol κ or a polκ modulator, but with altered and even improved characteristics.

3. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

4. Protein Purification

It may be desirable to purify pol κ, a pol κ modulator, or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

5. Antibodies

Another embodiment of the present invention are antibodies, in some cases, a human monoclonal antibody, immunoreactive with the polypeptide sequence of pol κ (SEQ ID NO:2). It is understood that antibodies can be used for inhibiting or modulating pol κ. It is also understood that this antibody is useful for screening samples from human patients for the purpose of detecting pol κ present in the samples. The antibody also may be useful in the screening of expressed DNA segments or peptides and proteins for the discovery of related antigenic sequences. In addition, the antibody may be useful in passive immunotherapy for cancer. All such uses of the said antibody and any antigens or epitopic sequences so discovered fall within the scope of the present invention.

a. Antibody Generation

In certain embodiments, the present invention involves antibodies. For example, all or part of a monoclonal, single chain, or humanized antibody may function as a modulator of pol κ. Other aspects of the invention involve administering antibodies as a form of treatment or as a diagnostic to identify or quantify a particular polypeptide, such as pol κ. As detailed above, in addition to antibodies generated against full length proteins, antibodies also may be generated in response to smaller constructs comprising epitopic core regions, including wild-type and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (mAbs) are recognized to have certain advantages, e.g., reproducibility and large-scale production, and their use is generally preferred. The invention thus provides monoclonal antibodies of the human, murine, monkey, rat, hamster, rabbit and even chicken origin.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow and Lane, “Antibodies: A Laboratory Manual,” Cold Spring Harbor Laboratory, 1988; incorporated herein by reference).

The methods for generating monoclonal antibodies (mAbs) generally begin along the same lines as those for preparing polyclonal antibodies. Briefly, a polyclonal antibody may be prepared by immunizing an animal with an immunogenic polypeptide composition in accordance with the present invention and collecting antisera from that immunized animal. Alternatively, in some embodiments of the present invention, serum is collected from persons who may have been exposed to a particular antigen. Exposure to a particular antigen may occur a work environment, such that those persons have been occupationally exposed to a particular antigen and have developed polyclonal antibodies to a peptide, polypeptide, or protein. In some embodiments of the invention polyclonal serum from occupationally exposed persons is used to identify antigenic regions in the gelonin toxin through the use of immunodetection methods.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/ Mead, NJ), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection also may be given. The process of boosting and titering is repeated until a suitable titer is achieved. When a desired level of immunogenicity is obtained, the immunized animal can be bled and the serum isolated and stored, and/or the animal can be used to generate mAbs.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

mAbs may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate mAbs. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions.

“Humanized” antibodies are also contemplated, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope. Examples of other teachings in this area include U.S. Pat. Nos. 6,054,297; 5,861,155; and 6,020,192, all specifically incorporated by reference. Methods for the development of antibodies that are “custom-tailored” to the patient's disease are likewise known and such custom-tailored antibodies are also contemplated.

b. Pol κ Antigenic Sequences

As another way of effecting modulation of pol κ in a subject, peptides corresponding to one or more antigenic determinants of the pol κ polypeptides of the present invention also can be prepared so that an immune response against pol κ is raised. Thus, it is contemplated that vaccination with an pol κ peptide or polypeptide may generate an autoimmune response in an immunized animal such that autoantibodies that specifically recognize the animal's endogenous pol κ protein. This vaccination technology is shown in U.S. Pat. Nos. 6,027,727; 5,785,970, and 5,609,870, which are hereby incorporated by reference.

Such peptides should generally be at least five or six amino acid residues in length and will preferably be about 10, 11, 12, 13, 14, 15, 16, 17, 18, 19, 20, 25 or about 30 amino acid residues in length, and may contain up to about 35-50 residues. For example, these peptides may comprise a pol κ amino acid sequence, such as 5, 6, 7, 8, 9, 10, 11, 12, 13, 14, 15, 16, 17, 18, 19, 20, 21, 22, 23, 24, 25, 30, 35, 40, 45, and 50 or more contiguous amino acids from SEQ ID NO:2. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means.

U.S. Pat. No. 4,554,101, incorporated herein by reference, teaches the identification and preparation of epitopes from primary amino acid sequences on the basis of hydrophilicity. Through the methods disclosed in Hopp, one of skill in the art would be able to identify epitopes from within an amino acid sequence such as the IGFBP-2 sequence disclosed herein in SEQ ID NO:2.

Numerous scientific publications have also been devoted to the prediction of secondary structure, and to the identification of epitopes, from analyses of amino acid sequences (Chou & Fasman, 1974a, b; 1978a, b; 1979). Any of these may be used, if desired, to supplement the teachings of Hopp in U.S. Pat. No. 4,554,101.

Moreover, computer programs are currently available to assist with predicting antigenic portions and epitopic core regions of proteins. Examples include those programs based upon the Jameson-Wolf analysis (Jameson & Wolf, 1988; Wolf et al., 1988), the program PepPlot® (Brutlag et al., 1990; Weinberger et al., 1985), and other new programs for protein tertiary structure prediction (Fetrow & Bryant, 1993). Another commercially available software program capable of carrying out such analyses is Mac Vector (IBI, New Haven, Conn.).

In further embodiments, major antigenic determinants of a pol κ polypeptide may be identified by an empirical approach in which portions of the gene encoding the pol κ polypeptide are expressed in a recombinant host, and the resulting proteins tested for their ability to elicit an immune response. For example, PCR™ can be used to prepare a range of peptides lacking successively longer fragments of the C-terminus of the protein. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTs™ system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

Once one or more such analyses are completed, polypeptides are prepared that contain at least the essential features of one or more antigenic determinants. The peptides are then employed in the generation of antisera against the polypeptide. Minigenes or gene fusions encoding these determinants also can be constructed and inserted into expression vectors by standard methods, for example, using PCR™ cloning methodology.

The use of such small peptides for antibody generation or vaccination typically requires conjugation of the peptide to an immunogenic carrier protein, such as hepatitis B surface antigen, keyhole limpet hemocyanin or bovine serum albumin, or other adjuvants discussed above (adjuvenated peptide). Alum is an adjuvant that has proven sufficiently non-toxic for use in humans. Methods for performing this conjugation are well known in the art. Other immunopotentiating compounds are also contemplated for use with the compositions of the invention such as polysaccharides, including chitosan, which is described in U.S. Pat. No. 5,980,912, hereby incorporated by reference. Multiple (more than one) pol κ epitopes may be crosslinked to one another (e.g. polymerized). Alternatively, a nucleic acid sequence encoding an pol κ peptide or polypeptide may be combined with a nucleic acid sequence that heightens the immune response. Such fusion proteins may comprise part or all of a foreign (non-self) protein such as bacterial sequences, for example.

Antibody titers effective to achieve a response against endogenous pol κ will vary with the species of the vaccinated animal, as well as with the sequence of the administered peptide. However, effective titers may be readily determined, for example, by testing a panel of animals with varying doses of the specific antigen and measuring the induced titers of autoantibodies (or anti-self antibodies) by known techniques, such as ELISA assays, and then correlating the titers with IGFBP-2-related cancer characteristics, e.g., tumor growth or size.

One of ordinary skill would know various assays to determine whether an immune response against pol κ was generated. The phrase “immune response” includes both cellular and humoral immune responses. Various B lymphocyte and T lymphocyte assays are well known, such as ELISAs, cytotoxic T lymphocyte (CTL) assays, such as chromium release assays, proliferation assays using peripheral blood lymphocytes (PBL), tetramer assays, and cytokine production assays. See Benjamini et al., 1991, hereby incorporated by reference.

6. Immunodetection Methods

As discussed, in some embodiments, the present invention concerns immunodetection methods for binding, purifying, removing, quantifying and/or otherwise detecting biological components such as antigenic regions on polypeptides and peptides. The immunodetection methods of the present invention can be used to identify antigenic regions of a peptide, polypeptide, or protein that has therapeutic implications, particularly in reducing the immunogenicity or antigenicity of the peptide, polypeptide, or protein in a target subject.

Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle et al., 1999; Gulbis et al., 1993; De Jager et al., 1993; and Nakamura et al., 1987, each incorporated herein by reference.

In general, the immunobinding methods include obtaining a sample suspected of containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any ORF antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

a. ELISAs

As detailed above, immunoassays, in their most simple and/or direct sense, are binding assays. Certain preferred immunoassays are the various types of enzyme linked immunosorbent assays (ELISAs) and/or radioimnunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

b. Immunohistochemistry

The antibodies of the present invention may also be used in conjunction with both fresh-frozen and/or formalin-fixed, paraffin-embedded tissue blocks prepared for study by immunohistochemistry (IHC). For example, immunohistochemistry may be utilized to characterize pol κ or to evaluate the amount pol κ in a cell. The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 mg of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

7. Lipid Components and Moieties

In certain embodiments, the present invention concerns compositions comprising one or more lipids associated with a nucleic acid, an amino acid molecule, such as a peptide, or another small molecule compound. In any of the embodiment discussed herein, the molecule may be either pol κ or a pol κ modulator, for example a nucleic acid encoding all or part of either pol κ or a pol κ modulator, or alternatively, a amino acid molecule encoding all or part of pol κ modulator. A lipid is a substance that is characteristically insoluble in water and extractable with an organic solvent. Compounds than those specifically described herein are understood by one of skill in the art as lipids, and are encompassed by the compositions and methods of the present invention. A lipid component and a non-lipid may be attached to one another, either covalently or non-covalently.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof.

a. Lipid Types

A neutral fat may comprise a glycerol and/or a fatty acid. A typical glycerol is a three carbon alcohol. A fatty acid generally is a molecule comprising a carbon chain with an acidic moiety (e.g., carboxylic acid) at an end of the chain. The carbon chain may of a fatty acid may be of any length, however, it is preferred that the length of the carbon chain be of from at least 2, 3, 4, 5, 6, 7, 8, 9, 10, 11, 12, 13, 14, 15, 16, 17, 18, 19, 20, 21, 22, 23, 24, 25, 26, 27, 28, 29, to 30 or more carbon atoms, and any range derivable therein. An example of a range is from about 8 to about 16 carbon atoms in the chain portion of the fatty acid. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated. The fatty acid may be branched, though in embodiments of the present invention, it is unbranched.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phoshotidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

b. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

C. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

d. Lipid Composition Structures

A nucleic acid molecule or amino acid molecule, such as a peptide, associated with a lipid may be dispersed in a solution containing a lipid, dissolved with a lipid, emulsified with a lipid, mixed with a lipid, combined with a lipid, covalently bonded to a lipid, contained as a suspension in a lipid or otherwise associated with a lipid. A lipid or lipid/pol κ modulator-associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine(Gibco BRL)-pol κ modulator or Superfect (Qiagen)-pol κ modulator complex is also contemplated.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10%to about 35% phosphatidyl choline, and about 1% of a drug. Thus, it is contemplated that lipid compositions of the present invention may comprise any of the lipids, lipid types or other components in any combination or percentage range.

i. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modem Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

ii. Micelles

A lipid may be comprised in a micelle. A micelles is a cluster or aggregate of lipid compounds, generally in the form of a lipid monolayer, may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

e. Liposomes

In particular embodiments, a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In specific aspects, a lipid and/or pol κ modulator may be, for example, encapsulated in the aqueous interior of a liposome, interspersed within the lipid bilayer of a liposome, attached to a liposome via a linking molecule that is associated with both the liposome and the pol κ modulator, entrapped in a liposome, complexed with a liposome, etc.

i. Making Liposomes

A liposome used according to the present invention can be made by different methods, as would be known to one of ordinary skill in the art. For example, a phospholipid (Avanti Polar Lipids, Alabaster, Ala.), such as for example the neutral phospholipid dioleoylphosphatidylcholine (DOPC), is dissolved in tert-butanol. The lipid(s) is then mixed with the pol κ modulator, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the pol κ modulator is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster, 1983; Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of modulatory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000× g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiements, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomal/pol κ modulator or liposomes only of appropriate and uniform size, which are structurally stable and produce maximal activity. Such techniques are well-known to those of skill in the art (see, for example Martin, 1990).

Once manufactured, lipid structures can be used to encapsulate compounds that are toxic (e.g., chemotherapeutics) or labile (e.g., nucleic acids) when in circulation. Liposomal encapsulation has resulted in a lower toxicity and a longer serum half-life for such compounds (Gabizon et al., 1990).

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997) and it is contemplated that liposomes are prepared by these methods. Alternate methods of preparing lipid-based formulations for nucleic acid delivery are described (WO 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

ii. Liposome Targeting

Although targetting may be achieved by employing a particular peptide sequence, association of the pol κ modulator with a liposome may also improve biodistribution and other properties of the pol κ modulator. For example, liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980). Successful liposome-mediated gene transfer in rats after intravenous injection has also been accomplished (Nicolau et al., 1987).

It is contemplated that a liposome/pol κ modulator composition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

Targeted delivery is achieved by the addition of ligands without compromising the ability of these liposomes deliver large amounts of pol κ modulator. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

5. Biochemical Cross-Linkers

It can be considered as a general guideline that any biochemical cross-linker that is appropriate for use in an immunotoxin will also be of use in the present context, and additional linkers may also be considered to join proteinaceous compositions that include peptides and polypeptides of the present invention.

Cross-linking reagents are used to form molecular bridges that tie together functional groups of two different molecules, e.g., a stablizing and coagulating agent. To link two different proteins in a step-wise manner, hetero-bifunctional cross-linkers can be used that eliminate unwanted homopolymer formation. Examples of such cross-linkers can be found in Table 3.

Hetero-Bifunctional Cross-Linkers

| | | | |
|---|---|---|---|
| | | | Space Arm |
| | | | Length\after cross- |
| linker | Reactive Toward | Advantages and Applications | linking |

| | | | |
|---|---|---|---|
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo-SMCC | Primary amines | Stable maleimide reactive group | 11.6 A |
| | Sulfbydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo-NHS | Primary amines | Hapten-Carrier conjugation | 0 |
| | Carboxyl groups | | |
| ABH | Carbohydrates | Reacts with sugar groups | 11.9 A |
| | Nonselective | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It can therefore be seen that a targeted peptide composition will generally have, or be derivatized to have, a functional group available for cross-linking purposes. This requirement is not considered to be limiting in that a wide variety of groups can be used in this manner. For example, primary or secondary amine groups, hydrazide or hydrazine groups, carboxyl alcohol, phosphate, or alkylating groups may be used for binding or cross-linking. For a general overview of linking technology, one may wish to refer to Ghose & Blair (1987).

The spacer arm between the two reactive groups of a cross-linkers may have various length and chemical compositions. A longer spacer arm allows a better flexibility of the conjugate components while some particular components in the bridge (e.g., benzene group) may lend extra stability to the reactive group or an increased resistance of the chemical link to the action of various aspects (e.g., disulfide bond resistant to reducing agents). The use of peptide spacers, such as L-Leu-L-Ala-L-Leu-L-Ala, is also contemplated.

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagents for use in immunotoxins is SMPT, which is a bifunctional cross-linker containing a disulfide bond that is “sterically hindered” by an adjacent benzene ring and methyl groups. It is believed that stearic hindrance of the disulfide bond serves a function of protecting the bond from attack by thiolate anions such as glutathione which can be present in tissues and blood, and thereby help in preventing decoupling of the conjugate prior to the delivery of the attached agent to the tumor site. It is contemplated that the SMPT agent may also be used in connection with the bispecific coagulating ligands of this invention.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art.

Once conjugated, the peptide generally will be purified to separate the conjugate from unconjugated targeting agents or coagulants and from other contaminants. A large a number of purification techniques are available for use in providing conjugates of a sufficient degree of purity to render them clinically useful. Purification methods based upon size separation, such as gel filtration, gel permeation or high performance liquid chromatography, will generally be of most use. Other chromatographic techniques, such as Blue-Sepharose separation, may also be used.

In addition to chemical conjugation, a pol κ modulator or pol κ polypeptide, peptide, or antibody may be modified at the protein level. Included within the scope of the invention are IgA protein fragments or other derivatives or analogs that are differentially modified during or after translation, for example by glycosylation, acetylation, phosphorylation, amidation, derivatization by known protecting/blocking groups, and proteolytic cleavage. Any number of chemical modifications may be carried out by known techniques, including but not limited to specific chemical cleavage by cyanogen bromide, trypsin, chymotrypsin, papain, V8 protease, NaBH₄; acetylation, formylation, farnesylation, oxidation, reduction; metabolic synthesis in the presence of tunicamycin.

II. Nucleic Acid Molecules

A. Polynucleotides Encoding Native Proteins or Modified Proteins

The present invention concerns polynucleotides, isolatable from cells, that are free from total genomic DNA and that are capable of expressing all or part of a protein or polypeptide. The polynucleotide may encode a pol κ polypeptide or a pol κ modulator. Recombinant proteins can be purified from expressing cells to yield active proteins.

As used herein, the term “DNA segment” refers to a DNA molecule that has been isolated free of total genomic DNA of a particular species. Therefore, a DNA segment encoding a polypeptide refers to a DNA segment that contains wild-type, polymorphic, or mutant polypeptide-coding sequences yet is isolated away from, or purified free from, total mammalian or human genomic DNA. Included within the term “DNA segment” are a polypeptide or polypeptides, DNA segments smaller than a polypeptide, and recombinant vectors, including, for example, plasmids, cosmids, phage, viruses, and the like.

As used in this application, the term “pol κ polynucleotide” refers to a pol κ-encoding nucleic acid molecule that has been isolated free of total genomic nucleic acid. Therefore, a “polynucleotide encoding pol κ” refers to a DNA segment that contains wild-type (SEQ ID NO: 1), mutant, or polymorphic pol κ polypeptide-coding sequences isolated away from, or purified free from, total mammalian or human genomic DNA. Therefore, for example, when the present application refers to the function or activity of pol κ or a “pol κ polypeptide,” it is meant that the polynucleotide encodes a molecule that has the polymerase activity of pol κ.

The term “cDNA” is intended to refer to DNA prepared using messenger RNA (MRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a particular polypeptide from a given species may be represented by natural variants that have slightly different nucleic acid sequences but, nonetheless, encode the same protein (see Table 2 above).

Similarly, a polynucleotide comprising an isolated or purified wild-type, polymorphic, or mutant polypeptide gene refers to a DNA segment including wild-type, polymorphic, or mutant polypeptide coding sequences and, in certain aspects, regulatory sequences, isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. A nucleic acid encoding all or part of a native or modified polypeptide may contain a contiguous nucleic acid sequence encoding all or a portion of such a polypeptide of the following lengths: about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400,410, 420, 430,440, 441,450, 460,470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs.

In particular embodiments, the invention concerns isolated DNA segments and recombinant vectors incorporating DNA sequences that encode a wild-type, polymorphic, or mutant pol κ or polκ modulator polypeptide or peptide that includes within its amino acid sequence a contiguous amino acid sequence in accordance with, or essentially corresponding to a native polypeptide. Thus, an isolated DNA segment or vector containing a DNA segment may encode, for example, a pol κ modulator that can inhibit or reduce pol κ activity. The term “recombinant” may be used in conjunction with a polypeptide or the name of a specific polypeptide, and this generally refers to a polypeptide produced from a nucleic acid molecule that has been manipulated in vitro or that is the replicated product of such a molecule.

In other embodiments, the invention concerns isolated DNA segments and recombinant vectors incorporating DNA sequences that encode a polypeptide or peptide that includes within its amino acid sequence a contiguous amino acid sequence in accordance with, or essentially corresponding to the polypeptide.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode full-length polypeptide from any source or encode a truncated version of the polypeptide, for example a truncated pol κ polypeptide, such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targetting or efficacy. As discussed above, a tag or other heterologous polypeptide may be added to the modified polypeptide-encoding sequence, wherein “heterologous” refers to a polypeptide that is not the same as the modified polypeptide.

In a non-limiting example, one or more nucleic acid constructs may be prepared that include a contiguous stretch of nucleotides identical to or complementary to the a particular gene, such as the DinB1 (human is SEQ ID NO:1) or dinB1 (mouse is SEQ ID NO:3) genes. A nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

The DNA segments used in the present invention encompass biologically functional equivalent modified polypeptides and peptides, for example, a modified gelonin toxin. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein, to reduce toxicity effects of the protein in vivo to a subject given the protein, or to increase the efficacy of any treatment involving the protein.

Sequence of an pol κ polypeptide will substantially correspond to a contiguous portion of that shown in SEQ ID NO:2, and have relatively few amino acids that are not identical to, or a biologically functional equivalent of, the amino acids shown in SEQ ID NO:2. The term “biologically functional equivalent” is well understood in the art and is further defined in detail herein.

Accordingly, sequences that have between about 70% and about 80%; or more preferably, between about 81% and about 90%; or even more preferably, between about 91% and about 99%; of amino acids that are identical or functionally equivalent to the amino acids of SEQ ID NO:2 will be sequences that are “essentially as set forth in SEQ ID NO:2.”

In certain other embodiments, the invention concerns isolated DNA segments and recombinant vectors that include within their sequence a contiguous nucleic acid sequence from that shown in SEQ ID NO:1 or SEQ ID NO:3. This definition is used in the same sense as described above and means that the nucleic acid sequence substantially corresponds to a contiguous portion of that shown in SEQ ID NO:1 and has relatively few codons that are not identical, or functionally equivalent, to the codons of SEQ ID NO:1. The term “functionally equivalent codon” is used herein to refer to codons that encode the same amino acid, such as the six codons for arginine or serine, and also refers to codons that encode biologically equivalent amino acids. See Table 4 below, which lists the codons preferred for use in humans, with the codons listed in decreasing order of preference from left to right in the table (Wada et al., 1990). Codon preferences for other organisms also are well known to those of skill in the art (Wada et al., 1990, included herein in its entirety by reference).

TABLE 4
Preferred Human DNA Codons

| Amino Acids | | | Codons |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | E | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG CTC TTG CTT CTA TTA |
| Methionine | Met | M | ATG |
| Asparagine | Asn | N | AAC AAT |
| Proline | Pro | P | CCC CCT CCA CCG |
| Glutamine | Gln | Q | CAG CAA |
| Arginine | Arg | R | CGC AGG CGG AGA CGA CGT |
| Serine | Ser | S | AGC TCC TCT AGT TCA TCG |
| Threonine | Thr | T | ACC ACA ACT ACG |
| Valine | Val | V | GTG GTC GTT GTA |
| Tryptophan | Trp | W | TGG |
| Tyrosine | Tyr | Y | TAC TAT |

The various probes and primers designed around the nucleotide sequences of the present invention may be of any length. By assigning numeric values to a sequence, for example, the first residue is 1, the second residue is 2, etc., an algorithm defining all primers can be proposed:

n to n+y

where n is an integer from 1 to the last number of the sequence and y is the length of the primer minus one, where n+y does not exceed the last number of the sequence. Thus, for a 10-mer, the probes correspond to bases 1 to 10, 2 to 11, 3 to 12 . . . and so on. For a 15-mer, the probes correspond to bases 1 to 15, 2 to 16, 3 to 17 . . . and so on. For a 20-mer, the probes correspond to bases 1 to 20, 2 to 21, 3 to 22 . . . and so on.

It also will be understood that this invention is not limited to the particular nucleic acid and amino acid sequences of SEQ ID NO:1, SEQ ID NO:2, SEQ ID NO:3, or SEQ ID NO:4. Recombinant vectors and isolated DNA segments may therefore variously include the pol κ coding regions themselves, coding regions bearing selected alterations or modifications in the basic coding region, or they may encode larger polypeptides that nevertheless include pol κ-coding regions or may encode biologically functional equivalent proteins or peptides that have variant amino acids sequences.

The DNA segments of the present invention encompass biologically functional equivalent pol κ proteins and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the antigenicity of the protein.

If desired, one also may prepare fusion proteins and peptides, e.g., where the pol κ- or pol κ modulator-coding regions are aligned within the same expression unit with other proteins or peptides having desired functions, such as for purification or immunodetection purposes (e.g., proteins that may be purified by affinity chromatography and enzyme label coding regions, respectively).

Encompassed by certain embodiments of the present invention are DNA segments encoding relatively small peptides, such as, for example, peptides of from about 15 to about 50 amino acids in length, and more preferably, of from about 15 to about 30 amino acids in length; and also larger polypeptides up to and including proteins corresponding to the full-length sequences set forth in SEQ ID NO:2 or SEQ ID NO:4, or to specific fragments of SEQ ID NO: 1 or SEQ ID NO:3 that correspond to differences as compared to the published sequence for pol κ.

1. Vectors

Native and modified polypeptides may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (1989) and Ausubel et al., 1996, both incorporated herein by reference. In addition to encoding a modified polypeptide such as modified gelonin, a vector may encode non-modified polypeptide sequences such as a tag or targetting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targetting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment, Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Table 5 lists several elements/promoters that may be employed, in the context of the present invention, to regulate the expression of a gene. This list is not intended to be exhaustive of all the possible elements involved in the promotion of expression but, merely, to be exemplary thereof. Table 6 provides examples of inducible elements, which are regions of a nucleic acid sequence that can be activated in response to a specific stimulus.

TABLE 5
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton |
| | et al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn |
| | et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek |
| | et al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake |
| | et al., 1988; Campbell et al., 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Chol et al., 1988; Reisman et al., |
| | 1989 |
| Papilloma Virus | Campo et al., 1983; Lusky et al., 1983; Spandidos and |
| | Wilkie, 1983; Spalholz et al., 1985; Lusky et al., 1986; |
| | Cripe et al., 1987; Gloss et al., 1987; Hirochika et al., |
| | 1987; Stephens et al., 1987 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits |
| | et al., 1988; Feng et al., 1988; Takebe et al., 1988; Rosen |
| | et al., 1988; Berkhout et al., 1989; Laspia et al., 1989; |
| | Sharp et al., Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 6
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa |
| | | et al., 1987, Karin et al., 1987; |
| | | Angel et al., 1987b; McNeall |
| | | et al., 1989 |
| MMTV (mouse mammary | Glucocorticoids | Huang et al., 1981; Lee et al., |
| tumor virus) | | 1981; Majors et al., 1983; |
| | | Chandler et at., 1983; Lee et al., |
| | | 1984; Ponta et al., 1985; Sakai |
| | | et al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 Large T | Taylor et al., 1989, 1990a, 1990b |
| | Antigen | |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996).

Also contemplated as useful in the present invention are the dectin-1 and dectin-2 promoters. Additional viral promoters, cellular promoters/enhancers and inducible promoters/enhancers that could be used in combination with the present invention are listed in Tables 5 and 6. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest. Alternatively, a tissue-specific promoter for cancer gene therapy (Table 7) or the targeting of tumors (Table 8) may be employed with the nucleic acid molecules of the present invention.

TABLE 7
Candidate Tissue-Specific Promoters for Cancer Gene Therapy

| | Cancers in which promoter | Normal cells in which |
|---|---|---|
| Tissue-specific promoter | is active | promoter is active |
| Carcinoembryonic antigen | Most colorectal carcinomas; | Colonic mucosa; gastric |
| (CEA)* | 50% of lung carcinomas; 40- | mucosa; lung epithelia; |
| | 50% of gastric carcinomas; | eccrine sweat glands; cells in |
| | most pancreatic carcinomas; | testes |
| | many breast carcinomas | |
| Prostate-specific antigen | Most prostate carcinomas | Prostate epithelium |
| (PSA) | | |
| Vasoactive intestinal peptide | Majority of non-small cell | Neurons; lymphocytes; mast |
| (VIP) | lung cancers | cells; eosinophils |
| Surfactant protein A (SP-A) | Many lung adenocarcinomas | Type II pneumocytes; Clara |
| | cells | |
| Human achacte-scute | Most small cell lung cancers | Neuroendocrine cells in lung |
| homolog (hASH) | | |
| Mucin-1 (MUC1)** | Most adenocarcinomas | Glandular epithelial cells in |
| | (originating from any tissue) | breast and in respiratory, |
| | | gastrointestinal, and |
| | | genitourinary tracts |
| Alpha-fetoprotein | Most hepatocellular | Hepatocytes (under certain |
| | carcinomas; possibly many | conditions); testis |
| | testicular cancers | |
| Albumin | Most hepatocellular | Hepatocytes |
| | carcinomas | |
| Tyrosinase | Most melanomas | Melanocytes; astrocytes; |
| | | Schwann cells; some neurons |
| Tyrosine-binding protein | Most melanomas | Melanocytes; astrocytes, |
| (TRP) | | Schwann cells; some neurons |
| Keratin 14 | Presumably many squamous | Keratinocytes |
| | cell carcinomas (e.g.: Head | |
| | and neck cancers) | |
| EBV LD-2 | Many squamous cell | Keratinocytes of upper |
| | carcinomas of head and neck | digestive Keratinocytes of |
| | | upper digestive tract |
| Glial fibrillary acidic protein | Many astrocytomas | Astrocytes |
| (GFAP) | | |
| Myelin basic protein (MBP) | Many gliomas | Oligodendrocytes |
| Testis-specific angiotensin- | Possibly many testicular | Spermatazoa |
| converting enzyme (Testis- | cancers | |
| specific ACE) | | |
| Osteocalcin | Possibly many osteosarcomas | Osteoblasts |

TABLE 8
Candidate Promoters for Use with a Tissue-Specific Targeting of Tumors

| | Cancers in which Promoter | Normal cells in which |
|---|---|---|
| Promoter | is active | Promoter is active |
| E2F-regulated promoter | Almost all cancers | Proliferating cells |
| HLA-G | Many colorectal carcinomas; | Lymphocytes; monocytes; |
| | many melanomas; possibly | spermatocytes; trophoblast |
| | many other cancers | |
| FasL | Most melanomas; many | Activated leukocytes: |
| | pancreatic carcinomas; most | neurons; endothelial cells; |
| | astrocytomas possibly many | keratinocytes; cells in |
| | other cancers | immunoprivileged tissues; |
| | | some cells in lungs, ovaries, |
| | | liver, and prostate |
| Myc-regulated promoter | Most lung carcinomas (both | Proliferating cells (only some |
| | small cell and non-small cell); | cell-types): mammary |
| | most colorectal carcinomas | epithelial cells (including non- |
| | | proliferating) |
| MAGE-1 | Many melanomas; some non- | Testis |
| | small cell lung carcinomas; | |
| | some breast carcinomas | |
| VEGF | 70% of all cancers | Cells at sites of |
| | (constitutive overexpression in | neovascularization (but unlike |
| | many cancers) | in tumors, expression is |
| | | transient, less strong, and |
| | | never constitutive) |
| bFGF | Presumably many different | Cells at sites of ischemia (but |
| | cancers, since bFGF | unlike tumors, expression is |
| | expression is induced by | transient, less strong, and |
| | ischemic conditions | never constitutive) |
| COX-2 | Most colorectal carcinomas; | Cells at sites of inflammation |
| | many lung carcinomas; | |
| | possibly many other cancers | |
| IL-10 | Most colorectal carcinomas; | Leukocytes |
| | many lung carcinomas; many | |
| | squamous cell carcinomas of | |
| | head and neck; possibly many | |
| | other cancers | |
| GRP78/BiP | Presumably many different | Cells at sites of ishemia |
| | cancers, since GRP7S | |
| | expression is induced by | |
| | tumor-specific conditions | |
| CarG elements from Egr-1 | Induced by ionization | Cells exposed to ionizing |
| | radiation, so conceivably most | radiation; leukocytes |
| | tumors upon irradiation | |

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

C. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

2. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

3. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAxBAc® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REx™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

4. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

5. Antisense and Ribozymes

Modulators of pol κ include molecules that directly affect RNA transcripts encoding pol κ polypeptides. Antisense and ribozyme molecules target a particular sequence to achieve a reduction or elimination of a particular polypeptide, such as pol κ. Thus, it is contemplated that nucleic acid molecules that are identical or complementary to all or part of SEQ ID NO:1 and SEQ ID NO:3 are included as part of the invention.

a. Antisense Molecules

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction may be used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

b. Ribozymes

The use of pol κ-specific ribozymes is claimed in the present application. The following information is provided in order to compliment the earlier section and to assist those of skill in the art in this endeavor.

Ribozymes are RNA-protein complexes that cleave nucleic acids in a site-specific fashion. Ribozymes have specific catalytic domains that possess endonuclease activity (Kim and Cech, 1987; Gerlack et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990; Sioud et al., 1992). Recently, it was reported that ribozymes elicited genetic changes in some cell lines to which they were applied; the altered genes included the oncogenes H-ras, c-fos and genes of HIV. Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Several different ribozyme motifs have been described with RNA cleavage activity (reviewed in Symons, 1992). Examples that would be expected to function equivalently for the down regulation of pol κ include sequences from the Group I self splicing introns including tobacco ringspot virus (Prody et al., 1986), avocado sunblotch viroid (Palukaitis et al., 1979; Symons, 1981), and Lucerne transient streak virus (Forster and Symons, 1987). Sequences from these and related viruses are referred to as hammerhead ribozymes based on a predicted folded secondary structure.

Other suitable ribozymes include sequences from RNase P with RNA cleavage activity (Yuan et al., 1992; Yuan and Altman, 1994), hairpin ribozyme structures (Berzal-Herranz et al., 1992; Chowrira et al., 1993) and hepatitis 6 virus based ribozymes (Perrotta and Been, 1992). The general design and optimization of ribozyme directed RNA cleavage activity has been discussed in detail (Haseloff and Gerlach, 1988; Symons, 1992; Chowrira, et al., 1994; and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16. Therefore, for a given target messenger RNA of 1000 bases, 187 dinucleotide cleavage sites are statistically possible. The message for IGFBP-2 targeted here are greater than 1400 bases long, with greater than 260 possible cleavage sites.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in pol κ-targeted ribozymes is simply a matter of preparing and testing a given sequence, and is a routinely practiced “screening” method known to those of skill in the art.

B. Nucleic Acid Detection

In addition to their use in directing the expression of pol κ modulator proteins, polypeptides and/or peptides, the nucleic acid sequences disclosed herein have a variety of other uses. For example, they have utility as probes or primers for embodiments involving nucleic acid hybridization. They may be used in diagnostic or screening methods of the present invention. Detection of nucleic acids encoding pol κ or pol κ modulators are encompassed by the invention.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mnM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to SEQ ID NO:1 or SEQ ID NO:3 or any other SEQ ID NO are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substititution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

a. Design and Theoretical Considerations for Relative Quantitative RT-PCR

Reverse transcription (RT) of RNA to cDNA followed by relative quantitative PCR (RT-PCR) can be used to determine the relative concentrations of specific mRNA species isolated from a cell, such as a pol κ-encoding transcript. By determining that the concentration of a specific mRNA species varies, it is shown that the gene encoding the specific mRNA species is differentially expressed.

In PCR, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

The second condition that must be met for an RT-PCR experiment to successfully determine the relative abundances of a particular mRNA species is that relative concentrations of the amplifiable cDNAs must be normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR assay for plant tissue. The problems inherent in plant tissue samples are that they are of variable quantity (making normalization problematic), and that they are of variable quality (necessitating the co-amplification of a reliable internal control, preferably of larger size than the target). Both of these problems are overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR assay with an external standard protocol. These assays sample the PCR products in the linear portion of their amplification curves. The number of PCR cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

One reason for this advantage is that without the internal standard/competitor, all of the reagents can be converted into a single PCR product in the linear range of the amplification curve, thus increasing the sensitivity of the assay. Another reason is that with only one PCR product, display of the product on an electrophoretic gel or another display method becomes less complex, has less background and is easier to interpret.

b. Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al., 1991). It is contemplated that this technology may be used in conjunction with evaluating the expression level of pol κ with respect to diagnostic, as well as preventative and treatment methods of the invention.

C. Methods of Gene Transfer

Suitable methods for nucleic acid delivery to effect expression of compositions of the present invention are believed to include virtually any method by which a nucleic acid (e.g., DNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

II. Screening Methods Involving Pol κ

A. Screening for Modulators of Pol κ

The present invention further comprises methods for identifying modulators of pol κ activity. These assays may comprise random screening of large libraries of candidate substances; alternatively, the assays may be used to focus on particular classes of compounds selected with an eye towards structural attributes that are believed to make them more likely to modulate the function of pol κ.

By function, it is meant that one may assay for a measurable effect on pol κ activity. To identify a pol κ modulator, one generally will determine the activity or level of inhibition of pol κ in the presence and absence of the candidate substance, wherein a modulator is defined as any substance that alters these characteristics. For example, a method generally comprises:

(a) providing a candidate modulator;

(b) admixing the candidate modulator with an isolated compound or cell expressing the compound;

(c) measuring one or more characteristics of the compound or cell in step (b); and

(d) comparing the characteristic measured in step (c) with the characteristic of the compound or cell in the absence of said candidate modulator,

wherein a difference between the measured characteristics indicates that said candidate modulator is, indeed, a modulator of the compound or cell.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may potentially inhibit or reduce pol κ activity or mutagenicity generally. The candidate substance may be a protein or fragment thereof, a small molecule, or even a nucleic acid molecule. An example of pharmacological compounds will be compounds that are structurally related to pol κ, or a substrate of pol κ, such as a nucleic acid molecule. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-diotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on pol κ. Regardless of the type of inhibitor or activator identified by the present screening methods, the effect of the inhibition or activator by such a compound results in alteration in pol κ activity as compared to that observed in the absence of the added candidate substance.

2. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

B. Diagnostic Methods

In some embodiments of the present invention, methods of screening for pol κ activity, expression level, and mutation status of the gene or transcript encoding pol κ maybe employed as a diagnostic method to identify subjects who have or may be at risk for developing cancer or other hyprproliferative diseases. Pol κ activity may be evaluated using any of the methods and compositions disclosed herein, including assays involving evaluating error rates, fidelity, processivity, and susceptibility to certain compounds that inhibit other polyermases. Any othe the compounds or methods described herein may be employed to implement these diagnostic methods.

Assays to evaluate the level of expression of a polypeptide are well known to those of skill in the art. This can be accomplished also by assaying pol κ mRNA levels, mRNA stability or turnover, as well as protein expression levels. It is further contemplated that any post-translational processing of pol κ may also be evaluated, as well as whether it is being localized or regulated properly. In some cases an antibody that specifically binds pol κ may be used.

Furthemore, it is contemplated that the status of the gene may be evaluated directly or indirectly, by evaluating genomic DNA sequence comprising the pol κ coding regions and noncoding regions (introns, and upstream and downstream sequences) or mRNA sequence. The invention also includes determining whether any polymorphisms exist in pol κ genomic sequences (coding and noncoding). Such assays may involve polynucleotide regions that are identical or complementary to pol κ genomic sequences, such as primers and probes described herein.

IV. Pharmaceutical Formulations, Delivery, and Treatment Regimens

In an embodiment of the present invention, a method of treatment for a hyperproliferative disease, such as cancer, by the delivery of a pol κ modulator is contemplated. Hyperproliferative diseases that are most likely to be treated in the present invention are those that result from mutations in an oncogene and/or the reduced expression of a wild-type protein in the hyperproliferative cells. An increase in pol κ expression or activity is considered to be related to the promotion or maintenance of unregulated growth control. Examples of hyperproliferative diseases contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, bladder cancer and any other hyperproliferative diseases that may be treated by altering the activity of pol κ.

An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease.

Preferably, patients will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

A. Administration

To kill cells, inhibit cell growth, inhibit metastasis, decrease tumor or tissue size and otherwise reverse or reduce the malignant phenotype of tumor cells, using the methods and compositions of the present invention, one would generally contact a hyperproliferative cell with the therapeutic compound such as a polypeptide or an expression construct encoding a polypeptide. The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a pol κ modulator or an pol-κ modulator-encoding construct. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells.

B. Injectable Compositions and Formulations

The preferred method for the delivery of an expression construct encoding all or part of a pol κ protein to hyperproliferative cells in the present invention is via intratumoral injection. However, the pharmaceutical compositions disclosed herein may alternatively be administered parenterally, intravenously, intradermally, intramuscularly, transdermally or even intraperitoneally as described in U.S. Pat. No. 5,543,158; U.S. Pat. No. 5,641,515 and U.S. Pat. No.5,399,363 (each specifically incorporated herein by reference in its entirety).

Injection of nucleic acid constructs may be delivered by syringe or any other method used for injection of a solution, as long as the expression construct can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vaccuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

C. Combination Treatments

In order to increase the effectiveness of a treatment with the compositions of the present invention, such as a pol κ modulator, or expression construct coding therefor, it may be desirable to combine these compositions with other agents effective in the treatment of hyperproliferative disease, such as anti-cancer agents, or with surgery. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that pol κ modulator therapy could be used similarly in conjunction with chemotherapeutic, radiotherapeutic, immunotherapeutic or other biological intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed; pol κ modulator is “A” and the secondary anti-cancer agent, such as radio- or chemotherapy, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of pol κ would provide therapeutic benefit in the treatment of cancer.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as mda-7 has been shown to enhance anti-tumor effects (Ju et al., 2000).

As discussed earlier, examples of immunotherapies currently under investigation or in use are immune adjuvants (e.g., Mycobacterium bovis, Plasmodium falciparum, dinitrochlorobenzene and aromatic compounds) (U.S. Pat. Nos. 5,801,005; U.S. Pat. No. 5,739,169; Hui and Hashimoto, 1998; Christodoulides et al., 1998), cytokine therapy (e.g., interferons α, β and γ; IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the pol κ-related therapies described herein.

i. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al (1988). The development of human monoclonal antibodies is described in further detail elsewhere in the specification.

ii. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

iii. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

d. Genes

In yet another embodiment, the secondary treatment is a gene therapy in which a therapeutic polynucleotide (or second therapeutic polynucleotide if a pol κ modulator is provided to a cell by providing a nucleic acid encoding the modulator) is administered before, after, or at the same time as a pol κ modulator is administered. Delivery of a vector encoding a pol κ modulator in conjunction with a second vector encoding one of the following gene products will have a combined anti-hyperproliferative effect on target tissues. Alternatively, a single vector encoding both genes may be used. A variety of proteins are encompassed within the invention, some of which are described below. Table 6 lists various genes that may be targeted for gene therapy of some form in combination with the present invention.

i. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

ii. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

iii. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fás/Fas ligand, DR4 or DR5/TRAIL (Apo-2 ligand) would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Studies from a number of investigators have demonstrated that tumor cells that are resistant to TRAIL can be sensitized by subtoxic concentrations of drugs/cytokines and the sensitized tumor cells are significantly killed by TRAIL. (Bonavida et al., 1999; Bonavida et al., 2000; Gliniak et al., 1999; Keane et al., 1999). Ad-mda7 treatment of cancer cells results in the up-regulation of mRNA for TRAIL and TRAIL receptors. Therefore, administration of the combination of Ad-mda7 with recombinant TRAIL can be used as a treatment to provide enhanced anti-tumor activity. Furthermore, the combination of chemotherapeutics, such as CPT-11 or doxorubicin, with TRAIL also lead to enhanced anti-tumor activity and an increase in apoptosis. The combination of Ad-mda7 with chemotherapeutics and radiation therapy, including DNA damaging agents, will also provide enhanced anti-tumor effects. Some of these effects may be mediated via up-regulation of TRAIL or cognate receptors, whereas others may not. For example, enhanced anti-tumor activity with the combinations of Ad-mda7 and tamoxifen or doxorubicin (adriamycin) has been observed. Neither tamoxifen nor adriamycin are known to up-regulate TRAIL or cognate receptors.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

TABLE 9
Oncogenes

| | | | |
|---|---|---|---|
| Gene | Source | Human Disease | Function |

Growth Factors

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma virus | PDGF B |

Receptor Tyrosine Kinases

| | | | |
|---|---|---|---|
| ERBB/HER | Avian erythroblastosis | Amplified, deleted | EGF/TGF-α/ |
| | virus; ALV promoter | Squamous cell | Amphiregulin/ |
| | insertion; amplified | Cancer; glioblastoma | Hetacellulin receptor |
| | human tumors | | |
| ERBB-2/NEU/HER-2 | Transfected from rat | Amplified breast, | Regulated by NDF/ |
| | Glioblastomas | Ovarian, gastric | Heregulin and EGF- |
| | | cancers | Related factors |
| FMS | SM feline sarcoma virus | | CSF-1 receptor |
| KIT | HZ feline sarcoma virus | | MGF/Steel receptor |
| | | | Hematopoieis |
| TRK | Transfection from | | NGF (nerve growth |
| | human colon cancer | | Factor) receptor |
| MET | Transfection from | | Scatter factor/HGF |
| | human osteosarcoma | | Receptor |
| RET | Translocations and point | Sporadic thyroid cancer; | Orphan receptor Tyr |
| | mutations | familial medullary | Kinase |
| | | thyroid cancer; | |
| | | multiple endocrine | |
| | | neoplasias 2A and 2B | |
| ROS | URII avian sarcoma | | Orphan receptor Tyr |
| | Virus | | Kinase |
| PDGF receptor | Translocation | Chronic | TEL(ETS-like |
| | | Myelomonocytic | transcription factor)/ |
| | | Leukemia | PDGF receptor gene |
| | | | Fusion |
| TGF-β receptor | | Colon carcinoma | |
| | | mismatch mutation | |
| | | target | |

NONRECEPTOR TYROSINE KINASES

| | | | |
|---|---|---|---|
| ABI. | Abelson Mul.V | Chronic myelogenous | Interact with RB, RNA |
| | | leukemia translocation | polymerase, CRK, |
| | | with BCR | CBL |
| FPS/FES | Avian Fujinami SV;GA | | |
| | FeSV | | |
| LCK | Mul.V (murine leukemia | | Src family; T cell |
| | virus) promoter | | signaling; interacts |
| | insertion | | CD4/CD8 T cells |
| SRC | Avian Rous sarcoma | | Membrane-associated |
| | Virus | | Tyr kinase with |
| | | | signaling function; |
| | | | activated by receptor |
| | | | kinases |
| YES | Avian Y73 virus | | Src family; signaling |

SER/THR PROTEIN KINASES

| | | |
|---|---|---|
| AKT | AKT8 murine retrovirus | Regulated by PI(3)K?; |
| | | regulate 70-kd S6 k? |
| MOS | Maloney murine SV | GVBD; cystostatic |
| | | factor; MAP kinase |
| | | kinase |
| PIM-1 | Promoter insertion | |
| | Mouse | |
| RAF/MIL | 3611 murine SV; MH2 | Signaling in RAS |
| | avian SV | Pathway |

MISCELLANEOUS CELL SURFACE

| | | | |
|---|---|---|---|
| APC | Tumor suppressor | Colon cancer | Interacts with catenins |
| DCC | Tumor suppressor | Colon cancer | CAM domains |
| E-cadherin | Candidate tumor | Breast cancer | Extracellular homotypic |
| | Suppressor | | binding; intracellular |
| | | | interacts with catenins |
| PTC/NBCCS | Tumor suppressor and | Nevoid basal cell cancer | 12 transmembrane |
| | Drosophilia homology | syndrome (Gorline | domain; signals |
| | | syndrome) | through Gli homogue |
| | | | CI to antagonize |
| | | | hedgehog pathway |
| TAN-1 Notch | Translocation | T-ALI. | Signaling |
| homologue | | | |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | Phosphorylated RING |
| | | | finger interact Abl |
| CRK | CT1010ASV | | Adapted SH2/SH3 |
| | | | interact Abl |
| DPC4 | Tumor suppressor | Pancreatic cancer | TGF-β-related signaling |
| | | | Pathway |
| MAS | Transfection and | | Possible angiotensin |
| | Tumorigenicity | | Receptor |
| NCK | | | Adaptor SH2/5H3 |

GUANINE NUCLEOTIDE EXCHANGERS AND BINDING PROTEINS

| | | | |
|---|---|---|---|
| BCR | | Translocated with ABL | Exchanger; protein |
| | | in CML | Kinase |
| DBL | Transfection | | Exchanger |
| GSP | | | |
| NF-1 | Hereditary tumor | Tumor suppressor | RAS GAP |
| | Suppressor | neurofibromatosis | |
| OST | Transfection | | Exchanger |
| Harvey-Kirsten, N-RAS | HaRat SV; Ki RaSV; | Point mutations in many | Signal cascade |
| | Balb-MoMuSV; | human tumors | |
| | Transfection | | |
| VAV | Transfection | | S112/S113; exchanger |

NUCLEAR PROTEINS AND TRANSCRIPTION FACTORS

| | | | |
|---|---|---|---|
| BRCA1 | Heritable suppressor | Mammary | Localization unsettled |
| | | cancer/ovarian cancer | |
| BRCA2 | Heritable suppressor | Mammary cancer | Function unknown |
| ERBA | Avian erythroblastosis | | Thyroid hormone |
| | Virus | | receptor (transcription) |
| ETS | Avian E26 virus | | DNA binding |
| EVII | MuLV promotor | AML | Transcription factor |
| | Insertion | | |
| FOS | FBI/FBR murine | | Transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGI/LIM | Translocation t(3:12) | Lipoma | Gene fusions high |
| | t(12:15) | | mobility group |
| | | | HMGI-C (XT-hook) |
| | | | and transcription factor |
| | | | LIM or acidic domain |
| JUN | ASV-17 | | Transcription factor |
| | | | AP-1 with FOS |
| MLL/VHRX + ELI/MEN | Translocation/fusion | Acute myeloid leukemia | Gene fusion of DNA- |
| | ELL with MLL | | binding and methyl |
| | Trithorax-like gene | | transferase MLL with |
| | | | ELI RNA p01 II |
| | | | elongation factor |
| MYB | Avian myeloblastosis | | DNA binding |
| | Virus | | |
| MYC | Avian MC29; | Burkitt's lymphoma | DNA binding with |
| | Translocation B-cell | | MAX partner; cyclin |
| | Lymphomas; promoter | | regulation; interact |
| | Insertion avian | | RB?; regulate |
| | leukosis | | apoptosis? |
| | Virus | | |
| N-MYC | Amplified | Neuroblastoma | |
| L-MYC | | Lung cancer | |
| REL | Avian | | NF-κB family |
| | Retriculoendotheliosis | | transcription factor |
| | Virus | | |
| SKI | Avian SKV77O | | Transcription factor |
| | Retrovirus | | |
| VHL | Heritable suppressor | Von Hippel-Landau | Negative regulator or |
| | | syndrome | elongin; transcriptional |
| | | | elongation complex |
| WT-1 | | Wilm's tumor | Transcription factor |

CELL CYCLE/DNA DAMAGE RESPONSE

| | | | |
|---|---|---|---|
| ATM | Hereditary disorder | Ataxia-telangiectasia | Protein/lipid kinase |
| | | | homology; DNA |
| | | | damage response |
| | | | upstream in P53 |
| | | | pathway |
| BCL-2 | Translocation | Follicular lymphoma | Apoptosis |
| FACC | Point mutation | Fanconi's anemia group | |
| | | C(predisposition | |
| | | leukemia | |
| FHIT | Fragile site 3p14.2 | Lung carcinoma | Histidine-triad-related |
| | | | diadenosine 5′,3 ″″- |
| | | | P¹.p⁴tetraphosphate |
| | | | asymmetric hydrolase |
| hMLI/MutL | | HNPCC | Mismatch repair; MutL |
| | | | Homologue |
| HMSH2/MutS | | HNPCC | Mismatch repair; MutS |
| | | | Homologue |
| HPMS1 | | HNPCC | Mismatch repair; Mutl |
| | | | Homologue |
| hPMS2 | | HNPCC | Mismatch repair; MutL |
| | | | Homologue |
| INK4/MTS1 | Adjacent INK-4B at | Candidate MTS1 | p16 CDK inhibitor |
| | 9p21; CDK complexes | suppressor and MLM | |
| | | melanoma gene | |
| INK4B/MTS2 | | Candidate suppressor | p15 CDK inhibitor |
| MDM-2 | Amplified | Sarcoma | Negative regulator p53 |
| p53 | Association with SV40 | Mutated >50% human | Transcription factor; |
| | T antigen | tumors, including | checkpoint control; |
| | | hereditary Li-Fraumeni | apoptosis |
| | | syndrome | |
| PRAD1/BCL1 | Translocation with | Parathyroid adenoma; | Cyclin D |
| | Parathyroid hormone | B-CLL | |
| | or IgG | | |
| RB | Hereditary | Retinoblastoma; | Interact cyclin/cdk; |
| | Retinoblastoma; | osteosarcoma; breast | regulate E2F |
| | Association with many | cancer; other sporadic | transcription factor |
| | DNA virus tumor | cancers | |
| | Antigens | | |
| XPA | | xeroderma | Excision repair; photo- |
| | | pigmentosum; skin | product recognition; |
| | | cancer predisposition | zinc finger |

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Cloning of Human and Mouse Homologs of E. coli DinB

A. Materials and Methods

1. Cloning and Sequencing of the Mouse Dinb1 and Human DINB1 Genes

Total RNA from mouse embryonic fibroblasts or mouse testis was used as a template for first strand cDNA synthesis using the Superscript Preamplification system (Life Technologies, MD) according to the manufacturer's directions. Degenerate primers were designed based on conserved sequences in the E. coli DinB and C. elegans F22B7.6 proteins. The degenerate primers capable of encoding C. elegans F22B7.6 amino acids 93-99 (YFAAVEM) (SEQ ID NO:5) and amino acids 289-296 (NKPNGQ(Y/F)V) (SEQ ID NO:6) were: DPH1C 5′-CGA ATT CTA YTT YGC NGC IGT NGARAT G-3′ (SEQ ID NO:7) and DPH4NC 5′-CGG GAT CCA CRW AYT GIC CRT TIG GYT TRT T-3′ (SEQ ID NO:8) where Y=C/T, N=A/C/G/T, I=inosine, R=A/G, W=A/T. PCR reactions were performed using AmpliTaq polymerase and conditions recommended by the manufacturer (Perkin-Elmer, CA). Touchdown PCR was performed with annealing at 60° C.-51° C. for 2 cycles each, and 50° C. for 22 cycles. Amplification from mouse cDNA with these primers resulted in a product of 700 bp.

This portion of the mouse Dinb1 gene was used to generate a random-primed probe for screening a mouse testis cDNA library and a human HeLa cell cDNA library. Two partial cDNA clones obtained from each library were sequenced. Multiple rounds of 5′ and 3′ RACE were used to extend the putative cDNA sequences of the mouse and human genes, using RACE kits obtained from Life Technologies (MD) according to the manufacturer's directions. In addition, IMAGE clones #2063393 (DINB1 EST¹ A1375146), #1311317 (Dinb1 EST AA920064), and #385429 (Dinb1 EST W62931), were purchased (Research Genetics, AL) and sequenced. PCR products were cloned into vectors pCRII (Invitrogen, CA) or pGEM-T Easy (Promega, WI) by T-overhang ligation.

2. Databases and Protein Sequence Analysis

The databases used were the non-redundant (NR) database of protein sequences and the database of nucleotide sequences of unfinished bacterial genomes at the NCBI, NIH. The NR database was searched using the gapped BLAST program and the PSI-BLAST program as described (Altschul et al., 1997, 1998). The PSI-BLAST program was normally run to convergence, with the e-value of 0.01 as the cut-off for including sequences in the profile. Multiple alignments were constructed using the ClustalX program (Altschul et al., 1997) and modified manually on the basis of the alignment generated by PSI-BLAST. For phylogenetic tree construction large inserts and ambiguously aligned regions were removed from the multiple alignment. Phylogenetic trees were constructed using the neighbor-joining method (Thompson et al., 1994) with 1000 bootstrap replications as implemented in the PHYLIP package (Saitou & Nei, 1987).

3. Chromosome Mapping and Fluorescence In Situ Hybridization (FISH)

PCR primers designed to produce a human-specific product from the 5′ end of the DINB1 gene were used to screen the NIGMS human/rodent somatic cell hybrid mapping panel #2. The sequences of the PCR primers were:

| | |
|---|---|
| forward, | |
| 5′-TGGATAGCACAAAGGAGAAGTGTG-3′ | (SEQ ID NO:9) |
| reverse, | |
| 5′-AATCTGGACCCCTTCGTGGCTTCC-3′ | (SEQ ID NO:10) |

Screening with the PCR primers above yielded a single clone designated pDJ487d14. FISH was performed as described (Felsentein, 1996) with biotinylated pDJ487d14 as the probe against normal male donor metaphase chromosomes from cells labeled with BrdU for the last 4.5 hours of culture (Tonk et al., 1996).

4. Northern Blot Analysis of DINB1 Expression

A human multiple tissue Northern blot II (Clontech, CA) containing 2 μg poly(A)⁺ RNA/lane was hybridized with a labeled random-primed human DINB1 cDNA probe (nucleotides 659-1454) according to the manufacturer's directions.

5. RT-PCR Analysis of DINB1 Expression

RT-PCR was performed on cDNAs from multiple human tissues using primers complementary to the 5′ and 3′ ends of the human ORF. The primers used were:

| | |
|---|---|
| hDinB-5′, 5′ GTG GAT CCG CCA TGG ATAGCA CAA AGG AGA AGT G 3′ | (SEQ ID NO:11). |
| hDinB-3′, 5′ CAT ACC CTT GAT ATA TTT TTT AAG TAG TCG ACC GCG GAT CCA T 3′ | (SEQ ID NO:12). |

The amount of cDNA used per reaction was as follows; 5 μl 100 ng/μl HeLa cell library cDNA, 2 μl 2-10 ng/μl testis cDNA (Origene, MD) and 5 μl 0.2 ng/μl each cDNA from human multiple cDNA panel I (Clontech, CA). PCR reactions were performed using 2.5 U Expand High Fidelity DNA polymerase according to the manufacturer's suggestions (Boehringer-Mannheim, Germany) and touchdown PCR as described earlier. Samples (20 μl) were analyzed on a 1% agarose gel in TBE buffer.

B. cDNA and Protein Sequences of Human and Mouse DinB Homologs

The human DINB1 sequence of 4074 nucleotides (GenBank accession # AF163570) contains an ORF of 2.6 kb, which can encode a protein of 870 amino acids with a predicted Mᵣ=99 kDa. The mouse Dinb1 gene sequence of 4263 nucleotides (GenBank accession # AF163571) contains an ORF of 2.55 kb, which can encode a protein of 852 amino acids with a predicted Mᵣ=96 kDa. The context of the translation initiation codon of the human DINB1 ORF (ACCAUGG) is a perfect match to the Kozak consensus sequence (Kozak, 1989). That of the mouse Dinb1 ORF (AUCAUGG) is also a good match, especially in the key −4 and +3 positions. The predicted ORFs of the mouse and human genes appear to be complete, since stop codons are present in all three reading frames upstream and downstream of the protein coding regions. Furthermore, the nucleotide sequence identity between the mouse and human genes decreases dramatically immediately outside the putative coding regions, suggesting that these sequences are within the UTRs.

The sequenced region of the human 3′ UTR contains a putative AAUAAA polyadenylation signal at nucleotide 3276, which is not always used as a transcriptional termination signal, since additional 3′ UTR sequence is present beyond this point (data not shown). Tissue-specific alternative polyadenylation using this signal might account for additional DINB1 transcripts observed in testis by Northern blotting. The human DINB1 3′ UTR also contains 6 copies of the pentanucleotide AUUUA; such AU-rich elements, called AREs, have been shown to play a role in destabilization of mRNAs (Sachs, 1993). The mouse cDNA is apparently complete since its size is consistent with the largest mRNA (4.4 kb) detected by Northern analysis. The 3′ UTR of the mouse Dinb1 gene contains a consensus AAUAAA polyadenylation sequence at position 4201, and has 10 copies of the AUUUA destabilization signal.

C. Domain Organization and Phylogenetic Analysis of the UmuC/DinB Superfamily

The predicted human and mouse DinB1 proteins are substantially hydrophilic (30% acidic/basic residues) and contain bipartite nuclear localization signals at their C-termini. The conserved portion of the UmuC/DinB superfamily, including the mammalian DinB homologs, consists of the N-terminal nucleotidyl transferase domain, two tandem HhH domains implicated in DNA binding. No sequence similarity between the DinB nucleotidyl transferase domain and other known nucleotidyl transferases/DNA polymerases (or any other enzymes) was detected. (The PSI-BLAST program was run to convergence with a liberal cut-off of E=0.1 for each member of the superfamily). However, the multiple alignment of the DinB homologs reveals the presence of two highly conserved motifs that center at an invariant DE doublet (motif 2) and an DXD signature (motif 1) present in most family members. Both residues of the invariant DE doublet are essential for the DNA polymerase activity of yeast DNA pol η (Johnson et al., 1999). Conserved negatively charged residues flanked by hydrophobic residues are a typical feature of many polymerases (Poch et al., 1989; Braithwaite & Ito, 1993), in which they coordinate divalent cations directly involved in catalysis (Zachikov et al., 1996; Satumo et al., 1998). By inference, a similar role appears likely for the conserved acidic residues of the UmuC/DinB superfamily.

The mammalian DinB homologs also contain a duplicated C2HC zinc cluster domain. This distinctive version of the zinc finger is present (in combination with other enzymatic and binding domains) in two characterized DNA repair proteins, yeast Snml (Richter et al., 1992) and Rad18 (Jones et al., 1988), as well as the ORC6 subunit of the yeast origin-recognition complex (Li & Herskowitz, 1993), and several uncharacterized proteins. The apparent orthologs of Snm1 from higher eukaryotes lack the C2HC zinc cluster (L. Aravind and E. V. Koonin, unpublished observations), underscoring the evolutionary mobility of this domain. Rad18 is a DNA-binding protein with two identifiable distinct domains, namely a RING finger and the C2HC zinc cluster (Jones et al., 1988; Bailly et al., 1997). Since RING domains typically are associated with specific protein-protein interactions (Borden & Freemont, 1996), it is possible that the zinc cluster is involved in protein-DNA binding. Hence, the mammalian homologs of DinB appear to possess two unrelated DNA-binding domains, the double-HhH domain and the zinc cluster. A C2H2 zinc finger unrelated to the zinc cluster is present in the human XPV protein and its fungal homologs, although S. cerevisiae Rad30 contains a degenerate version. This underscores the functional association of the UmuC/DinB superfamily nucleotidyl transferases with Zn-binding modules that are likely to provide additional contacts with DNA, and demonstrates the plasticity of domain organization of these proteins, a general feature of DNA repair proteins (Aravind et al., 1999).

The UmuC/DinB superfamily appears to be represented in all eukaryotes, but shows a patchy distribution in bacteria and has thus far been identified in only one archaeon, S. sofataricus (Kulaeva et al., 1996). Among bacteria this family is represented in all Gram⁻ bacteria and some Gram⁻ Proteobacteria, but not in other lineages thus far. Phylogenetic analysis of the UmuC/DinB superfamily reveals several distinct groups that are convincingly supported by the bootstrap test. These can be separated into four subfamilies exemplified by E. coli UmuC protein, E. coli DinB protein, S. cerevisia Rev1 protein and S. cerevisia Rad30 protein. The Rev1 and Rad30 subfamilies are exclusively eukaryotic, whereas the UmuC subfamily comprises only bacterial proteins (although this is not statistically as strongly supported as in the other families). The mouse and human DinB homologs belong to a branch which includes the bacterial DinB protein and its eukaryotic homologs from S. pombe and C. elegans, suggesting a mitochondrial origin for these eukaryotic genes, with subsequent fusion of the Zn-cluster and the C-terminal globular domains. The presence of N-terminal extensions in the eukaryotic proteins that could serve as mitochondrial import peptides is consistent with this interpretation. The phylogenetic position of the DinB homolog from Solfulobus is uncertain, and in general it is not possible to propose a definitive evolutionary scenario for this superfamily. Given the presence of the umuC-related mucB genes on plasmids and bacteriophage SPBc2 (Woodgate & Sedgwick, 1992), a major contribution of horizontal gene transfer to the current distribution of the UmuC/DinB superfamily appears likely.

D. Chromosomal Mapping of the Human DINBI Gene

PCR analysis of the NIGMS human/rodent somatic cell hybrid mapping panel #2 with primers specific for the human DINB1 cDNA yielded amplification products exclusively in the human control lanes, and in the lane for the human chromosome 5/rodent hybrid. FISH with PAC clone pDJ487d14 containing part of the DINB1 gene yielded a single site of hybridization at band Sq13.1, consistent with the results from the human/rodent hybrid panel screen. No cross-hybridization to other homologs was observed. DNA sequencing and PCR analysis demonstrated that the clone contains only the first two exons of the DINB1 gene, plus substantial upstream sequence.

E. Expression of Human DINB1

The predominant DINB1 transcript observed in human multiple tissue blots is approximately 5 kb and is present at low but varying amounts in all tissues examined. Expression of the DINB1 gene is highest in testis, with additional abundant transcripts of ˜3.2 and ˜4.4 kb in this tissue. Some of these transcripts might arise due to the alternative use of the polyadenylation signal at position 3276.

In order to determine whether alternative splicing occurs within the coding region, the human DINB1 ORF was amplified from cDNA from a number of human tissues. RT-PCR of HeLa cDNA consistently yielded a single product of 2613 bp identical to the full-length DINB1 ORF reported here. This 2613 bp product was also found in a variety of human tissues. In contrast, RT-PCR of human testis cDNA yielded three products (2613, 2344, and 1484 bp), consistent with possible alternative splicing within the DINB1 coding region. These cDNA products were cloned and the putative sites of alternative splicing mapped. In the case of human DINB1 both alternate transcripts are expected to result in frameshift mutations.

RT-PCR of mouse testis cDNA with primers to the 5′ and 3′ end of the Dinb1 coding region also results in three products. However, the deletions in the mouse Dinb1 alternate transcripts are in-frame and are expected to express distinct protein isoforms which retain a nuclear localization signal. Intriguingly, one of the mouse Dinb1 alternative splice products removes the C-terminal zinc clusters.

### EXAMPLE 2

### Translesion Synthesis by DNA Polymerase κ

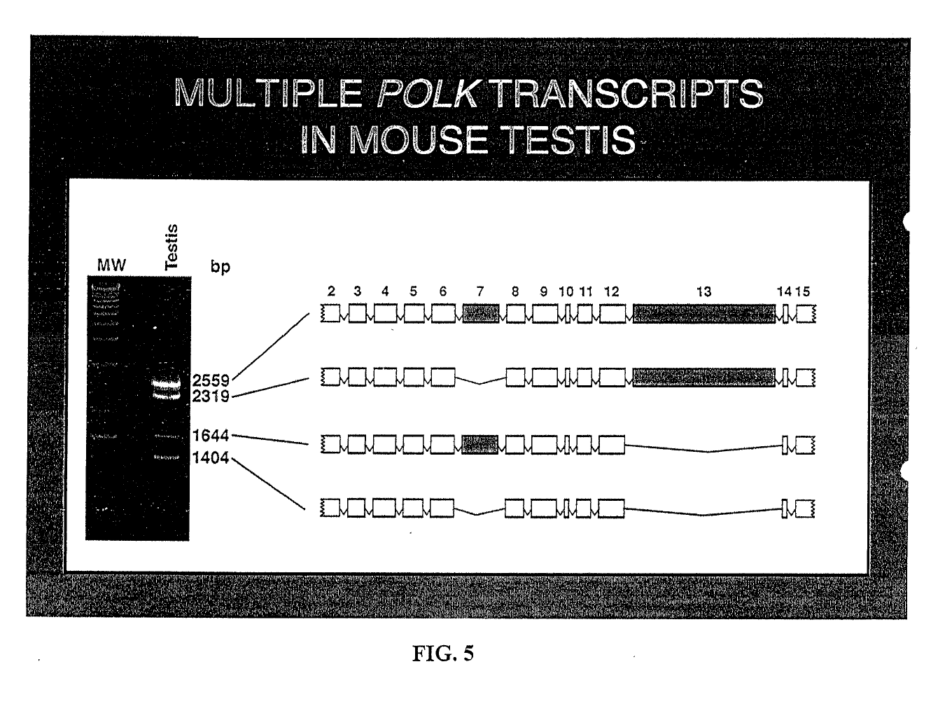

A number of the members of the UmuC/DinB superfamily have been implicated in DNA damage-induced mutagenesis and TLS. The definition of TLS utilizing simple in vitro primer extension systems requires cautious interpretation. A number of parameters may influence the outcome of such experiments including the assay conditions (pH, etc.), enzyme concentration, nucleotide concentration and even the template sequence context. Therefore, while lesion bypass in vitro may provide important clues to the activity of a DNA polymerase, these results must necessarily be correlated with in vivo studies. With these caveats in mind, human pol κ protein is unable to bypass thymine dimers, (6-4) photoproducts or abasic sites in vitro (Johnson et al., 2000). As shown in FIG. 5A, pol κ is also unable to bypass d[GpG-N7(1)-N7(2)] cisplatin intrastrand crosslinks, terminating synthesis 1 nucleotide prior to the lesion. However, the enzyme is able to weakly bypass N-(deoxyguanosin-8-yl) 2-(acetylamino) fluorene (G-AAF) adducts. The physiological relevance of this bypass is uncertain since it is observed only at high enzyme concentrations, and even under these conditions a significant fraction of the enzyme is arrested at the site of the lesion. Similar results have been independently reported by others (Ohashi et al., 2000b). It remains a formal possibility that pol κ is involved in TLS of a specific lesion(s) in DNA, as appears to be the case for pol η.

### EXAMPLE 3

### Expression of the Human POLK and Mouse Polk Genes

The predominant human POLK transcript is ˜5 kb in size (Gerlach et al., 1999). Northern blot and RT-PCR analyses has demonstrated that both the human and mouse POLK and Polk genes are ubiquitously expressed, but are expressed at particularly high levels in the testis (Gerlach et al., 1999). In addition, smaller alternative Polk/POLK transcripts are observed in mouse and human testis (Gerlach et al., 1999). In light of this observation we examined the cellular distribution of gene expression in mouse testis by in situ hybridization with a Polk antisense RNA probe. Expression was detected in mid-to late pachytene spermatocytes (which are still undergoing meiosis), as well as the post-meiotic round and elongating spermatids. Interstitial cells in the testis were negative.

The cell-specific expression of Polk transcripts in mouse testis hints at a potential role of the protein in spermatogenesis. Round and elongating spermatids are post-meiotic and post-mitotic cells. Hence, a role for a low fidelity DNA polymerase in such cells is not clearly evident. However, the duration of most of meiosis I and all of meiosis II is very brief relative to the very prolonged prophase of meiosis I. Interestingly, the related mouse Rad30b (POLI) gene is expressed in testis in a very similar pattern (McDonald et al., 1999), suggesting that the two proteins may have overlapping functions. The mouse Hr6b gene (the human homolog of the S. cerevisiae RAD6 gene), which encodes a ubiquitin-conjugating enzyme that has been implicated in chromatin remodeling, is also expressed at the same stage of spermatogenesis (Roest et al., 1996). Genetic studies have shown that the S. cerevisiae RAD30 gene is part of the RAD6 epistasis group (McDonald et al., 1997). In addition, male mice deleted for the Hr6B gene are sterile, suggesting a critical function of this gene in spermatogenesis (Roest et al., 1996).

### EXAMPLE 4

### Characterization of Pol κ, DNA Polymerase Encoded by Human DINB1 Gene

A. Materials and Methods

1. Media and Biochemical Reagent

Insect cell TMN-FH media was purchased from Pharmingen. The Klenow fragments of E. coli DNA polymerase I (exo⁺ and exo⁻) were obtained from New England Biolabs. Aphidicolin was from Sigma. Dideoxynucleotides were from United States Biochemicals. Glutathione-Sepharose was from Pharmacia. The protease inhibitor cocktail was purchased from Roche Molecular Biochemicals.

2. Expression of Wild-Type and Mutant GST/polκ

The human DINB1 open reading frame was amplified by high-fidelity polymerase chain reaction (PCR) using HeLa cell cDNA as template with primers HDinB5′ (5′-GTGGATCCGCCATGGATAGCACAAAGGAGAAGTG-3′) (SEQ ID NO:13) and HDinB3′-His6 (5′-ATGGATCCGCGGTCGACTAATGGTGGTGATGATGGTGCTTAAAAAATATA TCAAGGGTATG-3′) (SEQ ID NO: 14) to introduce Bam HI restriction sites (underlined) on both the 5′ and 3′ ends of the amplified fragment as well as six histidine residues on the 3′ end. The PCR product was cloned into pGEM-T Easy (Promega) to generate pHDINB1-6His and sequenced to confirm the integrity of the coding region. The 2.6 kb Bam HI fragment containing the human DINB1 coding region was then cloned into the same site of pAcG2T (Pharmingen) to generate an in-frame fusion with the glutathione-S-transferase gene, generating plasmid pAcG2T/HDINB 1-6His.

The C-terminal deletion mutant was made by high-fidelity PCR with primers HDinB5′ and HDinB-Δ3′-6His (5′-ATGGATCCGCGGTCGACTAATGGTGGTGATG ATGGTGAGATCTACCCATAAGCCTTAATCTCA-3′) (SEQ ID NO:15) introducing a Bam HI restriction site (underlined) and six histidine residues onto the 3′ end of the amplified fragment and cloned into pGEM-T Easy (Promega) to give pHDINB1ΔC-6His. The DE198/199 to AA198/199 double mutation was introduced into pHDINB1-6His using the Tranformer site-directed mutagenesis kit (Clontech) and primers GTE-MluI/HindIII (5′-GAGCTCCCAAAGCTTTGGATGCAT-3′) (SEQ ID NO:16) and HDinB-DE->AA (5′-CCATGAGTCTTGCTGCAGCCTACTTG-3′) (SEQ ID NO:17), the latter introducing a Pst I restriction site (underlined) to give pHDINB1mut-6His. The Bam HI fragments from pHDINB1ΔC-6His and pHDINB1mut-6His were cloned into the same site of pAcG2T to give pAcG2T/HDINB1ΔC-6His and pAcG2T/HDINB1mut-6His.

These plasmids were co-transfected into SF9 cells with BaculoGold DNA using a BaculoGold transfection kit (Pharmingen). Expression of both wild-type and mutant GST/pol κ was assayed by immunoblotting with anti-GST antisera. Two rounds of amplification produced a high titer stock of recombinant virus expressing GST/pol κ. The multiplicity of infection yielding optimal expression of full-length fusion protein was determined empirically.

3. Purification of GST/pol κ

Both mutant pAcG2T constructs were co-transfected into Sf9 cells as described for the wild-type. Approximately 1×10⁸ virus-infected Sf9 cells were harvested 3 days after infection and lysed in 20 ml of Lysis Buffer I (1% Triton X-100/10 mM Tris-HCl(pH7.5)/10 mM Na₂HPO₄(pH7.5)/1 mM EDTA/5 mM β-mercaptoethanol/1× protease inhibitors) by incubation on ice for 10 min. Insoluble material was removed by centrifugation to give the cytoplasmic extract. The pellet was resuspended in 20 ml of Lysis Buffer I containing 500 mM NaCl, and incubated on ice for 10 min. Insoluble material was removed by centrifugation to generate nuclear extract. The nuclear extract was diluted two-fold and bound in batch to 500 μl of glutathione agarose for 2 hours at 4° C. The resin was harvested by centrifugation and most of the supernatant removed. The resin was resuspended in the remaining supernatant and transferred to a 10 ml disposable column (Bio-Rad) to collect the resin by gravity. The resin was washed with 5 ml of Lysis Buffer I containing 250 mM NaCl, followed by 5 ml of Wash Buffer II (10% glycerol/100 mM NaCl/20 mM Tris-HCl (pH7.5)/0.01% IPEPAL-630/5 mM β-mercaptoethanol/1× protease inhibitors). Bound protein was eluted with 3.5 ml Wash Buffer II containing 10 mM reduced glutathione, and collected in a total of 10 fractions of 350 μl each. GST/pol κ-containing fractions (determined by SDS-PAGE and immunoblotting) were aliquoted, frozen in liquid nitrogen and stored at −80° C. GST/pol κ DNA polymerase activity was stable to multiple rounds of freezing and thawing.

4. DNA Substrates

The oligonucleotide derived primer-templates used as substrates in the DNA polymerase assays (24/44; 25/44; 27/44; 30/44 and 31/44) were the same as those described by Wagner et al. (6). Primers were purified by denaturing polyacrylamide gel electrophoresis. Five pmol of each primer was 5′ end-labeled with T4 polynucleotide kinase in the presence of (γ-³²P)ATP and purified on Bio-Gel P2 (BioRad) spun columns equilibrated in STE (100 mM NaCl/10 mM Tris (pH8.0)/1 mM EDTA). The various labeled primers (100 μl) were annealed to the template in a ratio of 1:1.5 (primer:template) by heating to ˜95° C. for 5 min followed by slow cooling to room temperature.

5. DNA Polymerase Assays

Standard polymerase reactions (10 μl) were performed in 50 mM Tris-HCl (pH7.0)/5 mM MgCl₂/1 mM DTT/10 mM NaCl/1% glycerol with 100 μM dNTPs, 2 nM GST/pol κ and 5 nM primer-template for 5 min at 37° C. unless indicated otherwise. Reactions were terminated by the addition of 1 μl 0.5M EDTA, concentrated under vacuum and resuspended in 5 l loading dye (90% deionized formamide/0.1× TBE/0.03% bromophenol blue/0.03% xylene cyanole FF). Following denaturation at 95° C. for 2 min, products were resolved by electrophoresis on 12% polyacrylamide gels containing 8 M urea. Gels were dried under vacuum and exposed to film at room temperature.

B. Human DinB1 Protein is a DNA Polymerase

To determine whether the product of the human DINB1 gene is a DNA polymerase, we expressed and purified recombinant human DinB1 protein. Expression in both E. coli and the yeast Schizosaccaromyces pombe consistently resulted in low yields and/or degraded protein. However, we were able to express full-length hDinB1 protein fused to glutathione-S-transferase (GST) in insect cells using a baculovirus expression system. The recombinant GST/hDinB1 protein was purified to apparent physical homogeneity from nuclear extracts by affinity chromatography on glutathione-agarose. The purified GST/hDinB 1 fraction contained primarily full-length fusion protein; however, some degradation products, including free GST, were observed and confirmed by immunoblotting with anti-GST antisera.

To test for DNA polymerase activity, various 5′-³²P end-labeled oligonucleotide primers were annealed to a 44 nucleotide template and used as substrates. In the presence of dNTPs and Mg⁺², the Klenow fragment of E. coli DNA polymerase I efficiently extended the primer to generate the expected 44 nucleotide product. Purified GST/hDinB1 protein also extended the primer, demonstrating an intrinsic DNA polymerase activity. The human DinB1 protein should be renamed as DNA polymerase kappa (pol κ) and the gene encoding it, POLK, in accordance with standard nomenclature for eukaryotic DNA polymerases (8,9). This designation has been approved by the human genome organization nomenclature committee (http://www.gene.ucl.ac.uk/nomenclature).

GST protein alone, or a purified (by the same procedure) GST/hDinB 1 mutant protein in which the conserved amino acid residues D198 and E199 were changed to alanine, was devoid of detectable DNA polymerase activity, indicating that the observed polymerase activity is intrinsic to the human DinB1 protein. In addition, a truncated GST/hDinB1 fusion protein lacking 360 amino acids at the C-terminus (GST/hDinB1ΔC) did not demonstrate DNA polymerase activity, indicating that sequences within this less highly conserved portion of the protein are required for activity.

A series of experiments was performed to determine the optimal conditions for pol κ DNA polymerase activity in vitro. GST/pol κ was most active over the pH range of 6.5-7.5, with reactions carried out at 37° C. To investigate the effect of ionic strength on DNA synthesis, increasing amounts of NaCl were added to the reactions. GST/pol κ activity was relatively insensitive to NaCl concentration up to 50 mM, but was significantly inhibited at salt concentrations of 100 mM or higher. As expected, a metal cofactor was required for activity. Either Mg⁺² or Mn⁺² was utilized, with the former being preferred. Based on these observations, all subsequent DNA polymerase assays using GST/pol κ were performed at pH 7.0 and 37° C. in the presence of Mg⁺². A time course of DNA polymerase activity showed that the majority of DNA synthesis by GST/pol κ under the conditions just described occurs within 5 min.

The range of incomplete extension products produced by GST/pol κ in the experiments described above suggested that human pol κ is endowed with limited or moderate processivity, as has also been observed for the E. coli DinB protein (Wagner et al., 1999). Whether purified human PCNA, a factor known to stimulate the processivity of the replicative DNA polymerases pol δ and pol ε (Weissbach et al., 1975; Burgers et al., 1990; Tang et al., 2000), increases the extent of DNA synthesis by GST/pol κ was examined. Addition of recombinant human PCNA had no detectable effect on GST/pol κ activity. The PCNA used in this experiment was shown to be active for stimulation of pol δ activity.

C. Pol κ is a Template-Directed DNA Polymerase Lacking 3′→5′ Proofreading Exonuclease Activity

To demonstrate that GST/pol κ is a template-directed DNA polymerase we performed polymerase assays in the presence of single deoxyribonucleotide triphosphates (dNTPs) on four different primer-templates, each designed to test for the correct incorporation of a particular dNTP. Under the single set of conditions tested GST/pol κ preferentially incorporated the correct nucleotide on each template. However, in all cases significant levels of misincorporation were also observed. For example, on the 27/44 primer-template GST/pol κ primarily catalyzed the accurate incorporation of dGTP as the first nucleotide, but also supported misincorporation of dATP and to a lesser extent dCTP. It was also observed that the level of GST/pol κ activity on the 24/44 substrate was significantly lower than on the other primer-templates.

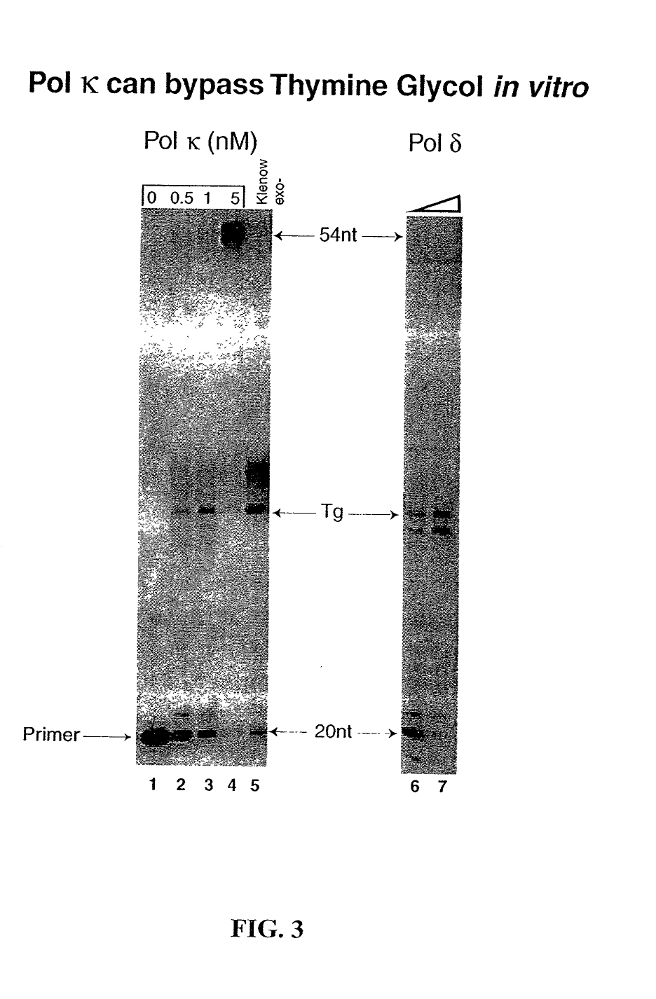

Given the detectable levels of nucleotide misincorporation observed in FIG. 3A, GST/pol κ was tested for 3′→5′ proofreading exonuclease activity. Using a substrate in which the 3′ nucleotide of the primer was not base paired with the template, no shortening of the primer by GST/pol κ or Klenow (exo⁻) was observed in the absence of dNTPs. In contrast, Klenow (exo^{'}) enzyme readily cleaved the primer. In the presence of dNTPs, the primer could only be efficiently extended by Klenow (exo⁺) following cleavage of the mispaired base. Limited extension by GST/pol κ was also observed from the 3′ mispaired primer. The low level of primer extended by Klenow (exo⁻) yielded a product 45 nucleotides in length due to incorporation of an additional dATP in a template-independent fashion (Prelich et al., 1987). This nucleotide would normally be removed by the 3′→5′ exonuclease activity of Klenow (exo⁺). The high levels of misincorporation together with the observed lack of a proofreading exonuclease activity suggest that pol κ is endowed with a low level of fidelity during synthesis of DNA.

GST/pol κ was tested for sensitivity to aphidicolin and dideoxynucleotides (ddNTPs), compounds known to inhibit other eukaryotic DNA polymerases to varying extents (McConnell et al., 1996). GST/pol κ activity was not inhibited by either aphidicolin or any of the ddNTPs used. The lack of sensitivity of pol κ to aphidicolin and ddNTPs is similar to that observed for human pol η (Hindges & Hübscher, 1997).

### EXAMPLE 5

### Fidelity and Processivity of DNA Synthesis by Human DNA Pol κ

A. Materials and Methods

1. Materials

All materials for the fidelity assay were from previously described sources (Bebenek et al., 1993). Human pol κ was expressed and purified as a full-length 870 amino acid polymerase fused to GST on the N-terminus and to hexahistidine on the C-terminus (Feaver, 2000). This was referred to as “full-length pol κ.” Pol κ was also purified as a C-terminal hexahistidine-tagged, catalytically-active fragment comprised of amino acids 1-560 (Ohashi, 2000), which was referred to as pol κ_{1-560.} Neither pol κ preparation excised a nucleotide from a mismatched primer terminus. The amount of 3′→5′ exonuclease activity was calculated to be less than ≦2% of the intrinsic exonuclease activity of Klenow fragment of E. coli DNA polymerase I.

2. DNA Synthesis Reactions

Reactions (25 μl) contained 0.7 nM M13mp2 DNA with a 407-nucleotide gap (from nucleotide −216 through +191 of the lacZ gene), 40 mM Tris-HCl (pH 8.0), 10 mM MgCl₂, 10 mM dithiothreitol, 6.25 μg BSA, 60 mM KCl, 2.5% glycerol and 1000 μM dNTPs. Synthesis was initiated by adding 110 pol κ₁₋₅₆₀ or 70 nM full-length pol κ. Reactions were incubated at 37° for one hour and terminated by adding EDTA to 15 mM. Products were analyzed by agarose gel electrophoresis as described (Bebenek et al., 1995).

3. Forward Mutation Assay

DNA products of the reactions were examined for the frequency of lacZ mutants as previously described (Bebenek et al., 1993). DNA from independent mutant phage was sequenced to identify the errors made during gap-filling synthesis. Error rates were calculated in three different ways. The standard approach expresses the error rate as “errors per detectable nucleotide synthesized,” by considering only changes in the 275-nucleotide LacZ a-complementation sequence that yield detectable light blue or colorless M13 plaque phenotypes (Bebenek et al., 1995). However, since most of the lacZ mutants generated by pol κ contain multiple sequence changes that include both silent and phenotypically detectable changes, the error rate can also be described simply as the number of observed mutations divided by the total number of copied nucleotides that were sequenced. A third calculation was performed using all base substitutions found in lacZ mutants containing known detectable base substitution mutations. Error rates generally differed by less than two-fold when these three calculations were compared. The error rates shown in the tables use the second, simplest methods.

4. Processivity Analysis

Measurements were performed with M13mp2 single-stranded DNA primed at a 3:1 molar ratio with a 5′-³²P-labeled 15-mer complementary to nucleotides 106 through 120 of the LacZ gene (where +1 is the first transcribed nucleotide). Reactions with HIV-1 RT and exonuclease-deficient Klenow fragment pol were performed as previously described (Bell, 1997; Bebenek et al., 1995). Pol κ reactions (30 μl) were performed as described above but contained 5 nM template-primer and the enzyme concentrations described in FIG. 3. Reactions were incubated at 37°. Ten μl aliquots were removed at 5, 15 or 30 min. and mixed with 10 μl of 99% formamide, 5 mM EDTA, 0.1% xylene cyanole, and 0.1% bromophenol blue, DNA products were analyzed by electrophoresis in a 16% polyacrylamide gel, in parallel with products of DNA sequencing reactions using the same template. Product bands were quantified by phosphorimagery and the probability of terminating processive synthesis was calculated (Bebenek et al., 1995).

B. Average Fidelity of Human Pol κ

The fidelity of pol κ was determined using a forward mutation assay that scores a variety of substitution, addition and deletion errors during DNA synthesis to copy a 407-nucleotide template present as a single stranded gap in M13mp2 DNA (Bebenek, 1995). Correct polymerization to fill the gap produces DNA that yields blue M13 plaques, while errors are scored as light blue or colorless plaques. DNA synthesis by both full-length pol κ and pol κ₁₋₅₆₀ filled the 407-nucleotide gap. When the DNA products were introduced into E. coli the lacZ mutant frequency among the resulting M13 plaques was 34% for full-length/GST pol κ and 25% for pol κ₁₋₅₆₀. These lacZ mutant frequencies are 10-100-fold higher than those generated by eukaryotic pol β (Osheroff, 1999), pol α, pol δ or pol ε (Thomas, 1991), but similar to that observed with human pol η (Matsuda, 2000). The data indicate that full-length/GST pol κ and pol κ₁₋₅₆₀ have very low fidelity overall.

To determine the nature and number of polymerase errors DNA from independent lacZ mutants was isolated, and all 407 nucleotides in the gap were sequenced. The lacZ mutants generated by both full-length pol κ and pol κ₁₋₅₆₀ contained an average of 4.2 and 3.7 mutations per mutant clone (Table 10). A variety of different sequence changes were observed (Table 10), and these were distributed throughout the target sequence. The majority of sequence changes were single-base substitutions. Given the total number of template nucleotides analyzed, the single base substitution error rates of pol κ₁₋₅₆₀ and full-length/GST pol κ are respectively 7.4×10⁻³ and 5.8×10⁻³. (Table 11). The second most frequent errors were single-base deletions, which were generated at average rates of 1.6×10⁻³ and 1.8×10⁻³ by pol κ₁₋₅₆₀ and full-lengt/GST pol κ, respectively. When compared to the error rates of other mammalian DNA polymerases determined in this assay (Table 11), the pol κ error rates for both base substitutions and single-base deletions are intermediate between those of pol η and pol β, and are much higher than that of the polymerases that replicate the nuclear and mitochondrial genomes.

TABLE 10
Summary of sequence changes generated by human DNA pol κ

| | pol κ₁₋₅₆₀ | pol κ |
|---|---|---|
| Total lacZ mutants sequenced | 108 | 51 |
| Total bases sequenced | 43.956 | 20,757 |
| Total sequence changes | 450 | 188 |
| Changes per lacZ mutant | 4.2 | 3.7 |
| Single-base substitutions | 324 | 121 |
| Single-base deletions | 70 | 38 |
| Two-base deletions | 13 | 5 |
| Single-base additions | 26 | 16 |
| Other changes | 17 | 8 |

^{a}Other changes include tandem double base substitutions, substitution-addition and substitution-deletion errors, deletions of larger numbers of nucleotides and complex errors.

TABLE 11
Single-base substitution and single-base deletion error rates
of pol κ compared to other eukaryotic DNA polymerases

| | | Error Rate (×10⁻⁵) | |
|---|---|---|---|
| DNA Polymerase | Family | Substitution | Deletion |
| Pol η | RAD30 | 3500 | 240 |
| Pol κ₁₋₅₆₀ | DINB | 740 | 160 |
| Pol κ (full-length) | DINB | 580 | 180 |
| Pol β | Pol X | 67 | 13 |
| Pol α | Pol B | 16 | 5 |
| Pol δ | Pol B | ˜1 | 2 |
| Pol ε | Pol B | ≦1 | ≦1 |
| Pol γ | Pol A | ≦1 | ≦1 |

Error rates for pol η are from Matsuda, 2000; for pol κ are from this study; and for the other polymerases are from Roberts, 1995.

C. Error Specificity

The error rates mentioned above are average rates for all 407 template nucleotides copied. Rates for individual subsets of errors were considered. Both pol κ₁₋₅₆₀ and full-length/GST pol κ generated all 12 possible base substitutions. Base substitution error rates (Table 12) were similar for the two forms of pol κ examined and varied between 0.2×10⁻³ (C.dCMP) and 8.2×10⁻³ (T.dCMP). Although mismatch-dependent variations are typical of all DNA polymerases studied to date (reviewed in Kunkel, 2000), the pol κ base substitution specificity is unusual in that the highest error rate is observed for the T.dCMP mispair (Table 12). In contrast, other DNA polymerases generate the T.dGMP mismatch at the highest rate (Matsuda, 2000; Thomas, 1991). From this bias and less apparent differences in the proportions of other substitutions, the ratio of misinsertion of pyrimidine dNTPs compared to purine dNTPs (from Table 12) is 60:40 for pol κ. This misinsertion bias is different from that of other DNA polymerases, whose general preference is to misinsert purine dNTPs (Kunkel, 1986; Matsuda, 2000; Thomas, 1991).

Analysis of the distribution of the single-base deletions within the 407-nucleotide target sequence also revealed sequence-dependent variations in deletion error rates. The deletion rate per template nucleotide copied is highest for loss of nucleotides within homopolymeric runs, and the highest rate is observed in the longest runs (Table 13). This suggests the formation of misaligned intermediates which are stabilized by correct base pairing. However, the rate is high even for deletion of non-iterated nucleotides (Table 13), such that there is only a 2- to 3-fold difference in error rate for loss of non-iterated nucleotides as compared to loss of nucleotides in homopolymeric runs of 4 and 5 bases. This difference is much smaller than that observed with several other DNA polymerases (for review, see Kunkel, 2000). As one example, note that the pol b deletion error rate in runs of 4 and 5 nucleotides is 35-fold higher than the rate for loss of non-iterated nucleotides (Table 13). As discussed below, these error specificity data suggest possible mechanisms of deletion by pol κ and have implications for spontaneous frameshift mutagenesis.

Both full-length/GST pol κ and pol κ₁₋₅₆₀ also frequently generate single-nucleotide additions (Table 10). Unexpectedly, many of these errors involve adding a nucleotide that is different from both of its neighbors. These include 10 of 14 additions of guanine between template nucleotides 5′-T and C-3′ and four of 20 additions of thymine between template nucleotides 5′-C/G/A and C-3′. This addition specificity is different than that of most other DNA polymerases, which typically add nucleotides to homopolymeric runs. This suggests that pol κ generates some addition errors by a mechanism other than classical strand slippage. Pol κ also produces two base deletions (Table 10), and these are also non-randomly distributed. Seven of 13 two-base deletions generated by pol κ₁₋₅₆₀ and four of five cases by full-length pol κ occurred at template 5′-GCT-3′ sites, where the template nucleotides C and T were deleted and the 5′-neighboring template base was a G. Among these, seven were at one location, nucleotides -58 and -59, which can therefore be considered a hot spot for this deletion by pol κ. Finally, “other” sequence changes were also observed (Table 10), including tandem double base substitutions, substitution-addition and substitution-deletion errors, deletions of larger numbers of nucleotides and complex errors.

TABLE 12
Base Substitution Error Rates of Human Pol κ

| Base | Mutation | Mispair | Pol κ₁₅₆₀ | | Pol κ | |
|---|---|---|---|---|---|---|
| (number) | From → To | Template•dNMP | Observed | Error Rate | Observed | Error Rate |
| A (99) | A→G | A•dCMP | 22 | 2.1 × 10⁻³ | 15 | 3.0 × 10⁻³ |
| | A→T | A•dAMP | 21 | 2.0 × 10⁻³ | 8 | 1.6 × 10⁻³ |
| | A→C | A•dGMP | 12 | 1.1 × 10⁻³ | 7 | 1.4 × 10⁻³ |
| T (91) | T→C | T•dGMP | 34 | 3.5 × 10⁻³ | 10 | 2.0 × 10⁻³ |
| | T→A | T•dTMP | 17 | 1.7 × 10⁻³ | 8 | 1.8 × 10⁻³ |
| | T→G | T•dCMP | 81 | 8.2 × 10⁻³ | 22 | 4.7 × 10⁻³ |
| G (95) | G→A | G•dTMP | 47 | 4.6 × 10⁻³ | 21 | 4.4 × 10⁻³ |
| | G→C | G•dGMP | 27 | 2.6 × 10⁻³ | 12 | 2.5 × 10⁻³ |
| | G→T | G•dAMP | 16 | 1.6 × 10⁻³ | 4 | 0.8 × 10⁻³ |
| C (122) | C→T | C•dAMP | 19 | 1.4 × 10⁻³ | 7 | 1.1 × 10⁻³ |
| | C→G | C•dCMP | 6 | 0.5 × 10⁻³ | 1 | 0.2 × 10⁻³ |
| | C→A | C•dTMP | 22 | 1.7 × 10⁻³ | 6 | 1.0 × 10⁻³ |

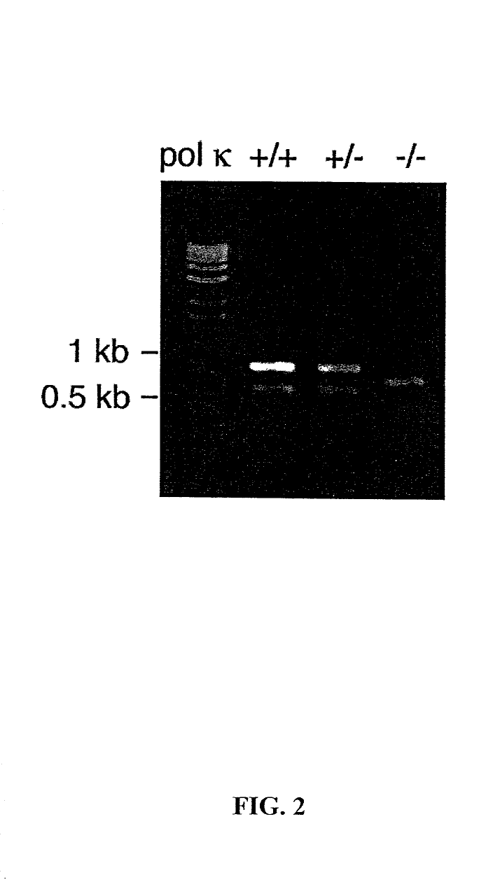

Error rates are the number of observed base substitutions (from FIG. 2) divided by the total number of A, T, G or C template nucleotides in the 407-base target (shown in parentheses in first column) among the 108 (pol 78₁₋₅₆₀) or 50 (full-length pol κ) lacZ clones sequenced.

TABLE 13
Sequence-Dependent Variations in Single-Base Deletion Error Rates of
Human Pol κ

| | | Pol κ₁₋₅₆₀ | | Pol κ | | Pol β |
|---|---|---|---|---|---|---|
| Run Length | | Observed | Error Rate | Observed | Error Rate | Error Rate |
| One | (204) | 23 | 1.0 × 10⁻³ | 12 | 1.2 × 10⁻³ | 0.02 × 10⁻³ |
| Two | (116) | 20 | 1.6 × 10⁻³ | 16 | 2.8 × 10⁻³ | 0.09 × 10⁻³ |
| Three | (57) | 17 | 2.8 × 10⁻³ | 6 | 2.1 × 10⁻³ | 0.23 × 10⁻³ |
| Four/Five | (30) | 10 | 3.1 × 10⁻³ | 4 | 2.7 × 10⁻³ | 0.70 × 10⁻³ |

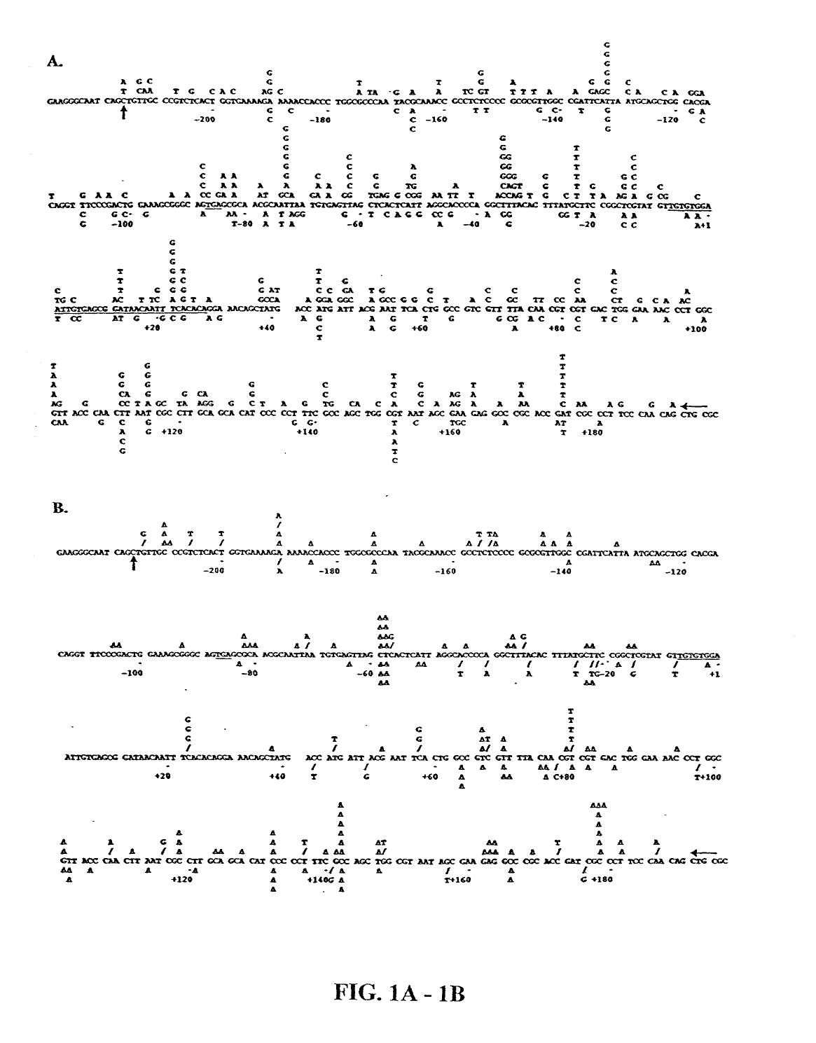

Error rates are the number of observed single-base deletions divided by the total number of template nucleotides present in runs of the lengths listed (shown in parentheses in first column) among 108 (pol κ₁₅₆₀) or 50 (full-length pol κ) lacZ clones sequenced. Error rates for pol β were calculated as previously described by considering only the phenotypically-detectable changes, using the data in FIG. 1 of Osheroff, 1999.

D. Processivity Analysis

The processivity of DNA synthesis, i.e., the number of nucleotides polymerized per cycle of polymerase association-dissociation, was evaluated. Primer extension reactions were performed using a large excess of template-primer over polymerase, such that once the polymerase completes a cycle of processive synthesis, the probability that the extended product is used again is negligible. Analysis of the products of the reaction catalyzed by pol κ₁₋₅₆₀ shows incorporation of one to five nucleotides. Quantification of band intensities reveals that 20 primers were extended per molecule of input pol κ₁₋₅₆₀, indicating that after terminating processive synthesis the polymerase dissociates and rebinds to a previously unused primer. The probability of termination following each incorporation event was calculated at between about 65 and 80%. Low processivity and high termination probabilities were also observed with two other template primers.

In contrast to these results, analysis of the products of the reaction catalyzed by full-length pol κ revealed incorporation of one to 76 nucleotides per cycle of pol κ association-dissociation. Thus, full-length pol κ is more processive than pol κ₁₋₅₆₀. The probability termination of processive synthesis by full-length enzyme varied by template position, from 46% at nucleotide 102 to 2.8% at nucleotide 81. A relatively intense band was observed corresponding to incorporation of the 76^{th} nucleotide. This is the beginning of the palindromic operator sequence in the LacZ gene, suggesting that full-length pol κ has difficulty polymerizing through a hairpin structure in the template.

When copying this same template sequence, Klenow fragment pol and HIV-1 RT have higher processivity (Bebenek et al., 1995; Bell et al., 1997). Full-length pol κ terminates processive synthesis more frequently than does Klenow fragment pol during incorporation of the first seven nucleotides, after which these two enzymes have somewhat different termination patterns. However, the termination probability and overall termination pattern of full-length pol κ is distinct from that of HIV-1 RT across the region scanned. Thus, the processivity of these three polymerases differs and is variably responsive to template sequence.

The concept of extensive synthesis by low fidelity pol κ is distinct from that proposed for human pol η, another polymerase in the UmuC/DinB nucleotidyl transferase superfamily. Pol η is encoded by the XPV gene (Masutani et al., 1999a; Masutani et al., 1999b; Johnson et al., 1999b), which is required to reduce UV radiation-induced mutations and hence suppress susceptibility to sunlight induced skin cancer, Pol η has low fidelity (Matsuda et al., 2000; Johnson et al., 2000b) and low processivity (Masutani et al., 2000), suggesting a model in which efficient bypass of template-distorting lesions is accomplished via relaxed geometric selectivity during incorporation of only a very few nucleotides. The intrinsically low processivity of pol η may limit its opportunity to generate synthesis errors and perhaps also allow a separate exonuclease to proofread any misinsertions that do occur. In this way, pol η promotes efficient lesion bypass and UV radiation-induced mutations are suppressed. The situation appears to be different for pol κ. Indeed, earlier studies have implicated the E. coli pol κ homolog DNA polymerase IV in untargeted mutagenesis of phage λ (Brotcorne-Lannoye et al., 1986), and overexpression of pol IV strongly enhanced spontaneous mutagenesis in E. coli cells transfe3cted with plasmids (Kim et al., 1997). When mouse pol κ was transiently expressed in cultured mouse cells, the spontaneous mutation rate was elevated about 10-fold (Ogi et al., 1999).

### EXAMPLE 6

### In Vivo Experiments in Mice

A. Generation of Mouse Strains Defective in Polk Expression and Mouse Strains That Overexpress Polk

To better understand the function of the mouse Polk gene in normal cells standard gene targeting disruption technologies are being used to knock-out the Polk gene (Ramírez-Solis et al., 1993; Rajewsky et al., 1996). The E. coli umuC and dinb genes, as well as the yeast Rev1 and RAD30 genes, are not essential, suggesting that a null Polk mouse is likely to be viable. However, two different approaches to generate Polk-defective mice will be taken, including one that will result in a conditional allele should the gene prove to be essential in mammals. It is predicted that Polk-deficient mice might manifest sensitization to the killing effects of DNA damage as well as reduced mutability and reduced cancer predisposition in response to DNA damage. In addition, if indeed the mouse Polk gene plays a role in somatic hypermutation of immunoglobulin genes, it is predicted that Polk-deficient mice would be defective in generating immunoglobulin diversity.

Since the biological functions of an error-prone DNA polymerase such as Polκ may be tightly regulated, overexpression of the mouse Polk gene might prove as informative as its absence, or even more so. Hence, the generation of strains of transgenic mice that overexpress the mouse Polk or human POLK cDNA are planned in order to assess whether they are more susceptible to spontaneous mutations and spontaneous tumors. Cells from such transgenic mice might also manifest enhanced resistance to the killing effects of DNA damage, as well as hypermutability in response to such agents. In addition, it is possible that an increase in the rate of DNA-damage induced tumors will be detected.

B. Strategy for the Generation of a Mouse Polk Knockout in Mouse Embryonic Stem Cells

Using classical gene targeting technology, no viable mice would be obtained if germline deletion of the mouse Polk gene were to be lethal. Furthermore, a limitation of classical gene targeting derives from the presence of the selectable marker in the targeted locus. Since the selectable marker must be active in order to allow ES cell selection, it is possible that its expression might alter the mutant phenotype in unpredictable ways. To avoid these potential complications a strategy to knock out the mouse Polk gene using the Cre-loxP recombination system is being pursued in collaboration with Klaus Rajewsky's laboratory at the University of Cologne, Germany (Rajewsky, 1996; Rajewsky et al., 1996).

Selected 5′ regions of the mouse Polk cDNA were used to screen a mouse genomic DNA library, resulting in the isolation of 4 genomic clones that were sequenced in their entirety. The intron/exon boundaries of the genomic region encompassing Polk exons 1-6, corresponding to the 5′ untranslated sequence and to the first 230 amino acids of the mouse Polκ protein have been identified. A targeting construct containing genomic sequence encompassing exon 6 of the Polk gene was made such that exon 6 is flanked by a loxP site on one side and a neomycin gene flanked by two loxP sites on the other side. Exon 6 contains the putative catalytic DE residues conserved in all members of the UmuC/DinB superfamily; therefore, deletion of this exon is predicted to result in a null protein. This flox-exon 6 targeting construct was introduced into ES cells by classical gene targeting techniques.

Basically, the targeting construct was introduced into low passage mouse 129 ES cells by electroporation followed by selection in G418-containing media. Correctly targeted traditional and conditional Polk knockout clones have been identified by Southern hybridization and the neomycin selection marker has been deleted by transient transfection with a Cre recombinase-encoding plasmid. This protocol yielded ES cell mutants in which exon 6 of the Polk gene was either deleted (total knockout) or was flanked by loxP sites (conditional knockout). Either mutation can be transmitted into the germline. In the former case, exon 6 of Polk will be deleted in all cells of the body, generating the equivalent of a classical knockout with the exception that no selectable marker gene remains in the mutant locus. In the latter case, the Polk mutant mice will carry a functional but loxP-flanked gene. ES cell clones that have been correctly targeted were microinjected into B6 blastocysts and implantated into pseudopregnant female mice. Resultant chimeric mice will be bred to determine germline transmission of the inactivated Polk gene.

Presently, chimeric mice containing the total or conditional Polk knockout alleles have been obtained and are being bred to determine whether germline transmission of the inactivated Polk gene has occurred using coat color as a marker. Once heterozygote strains have been established they will be bred to produce homozygous progeny for further study. Should the homozygous Polk null mouse prove inviable, conditional targeting of the Polk gene can then be achieved by crossing such mice with animals containing a Cre-transgene from which Cre recombinase is expressed in a cell-type-specific or inducible-manner. A variety of transgenic mice have been generated that express Cre-recombinase in an inducible or tissie-specific fashion, for example, using a promoter that is inducible by interferon alpha/beta or T-cell and B-cell specific promoters (Rajewsky, 1996).

C. Generation of Transgenic Mouse Strains That Overexpress the Mouse Polk and Human POLK Genes

It has been reported that transient overexpression of the mouse Polk (Dinb1) gene increases the number of mutations in mouse cells (Ogi et al., 1999). To test whether overexpression of the human POLK or mouse Polk gene in a multicellular organism increases the level of mutagenesis and perhaps leads to tumorigenesis, lines of transgenic mice that globally overexpress wild type mouse or human Polκ are being generated. One approach being taken is to overexpress the human POLK cDNA under control of a constitutive promoter (pCAG-POLK). The DNA fragments containing the pCAG-POLK sequences has been purified away from the remaining vector DNA and is ready for injection into the pronuclei of mouse eggs (see below).

However, given that overexpression of the pol κ protein might increase mutagenesis, it is reasonable to expect that introduction of a Polk-overexpressing transgene into mice may result in embryonic lethality or sterility. Further complications could also arise if transgenic founder embryos with weak expression of the transgene are the only ones to survive and are therefore selected, creating the potential for erroneous interpretations of the effects of overexpression of the Polk gene. To avoid these potential difficulties, use of a transgene construct that allows for regulated overexpression of the mouse Polk gene is planned by making use of the Cre/loxP system described earlier. The LacZ gene of the pCAG-CAT-LacZ transgene vector (Araki et al., 1995), will be replaced by a mouse Polk cDNA sequence containing an SV40 polyadenylation signal. The mouse Polk cDNA is currently being cloned into this transgene vector. This strategy will allow the mouse Polk transgene to be introduced into mice in a “silenced” form, since the loxP-flanked chloramphenicol acetyltransferase (CAT) reporter gene will be positioned between the CAG promoter and the Polk coding sequence, thus preventing its transcription. Expression levels of the CAT gene can then be used for the selection of transgenic founder lines with the highest expression in a wide variety of tissues. The Polk transgene can be “reactivated” by mating mice from these founder lines with mice of another transgenic line which express Cre recombinase. A homozygous transgenic mouse strain expressing the Cre gene from the CAG promoter is readily available. The ubiquitously active CAG (cyto-megalovirus immediate-early-enhancer/chicken p-actin hybrid) promoter has previously been shown to drive high levels of expression of the LacZ transgene in a wide variety of tissues (Sakai and Miyazaki, 1997).

The purified DNA fragments containing the pCAG-POLK and pCAG-CAT-Polk sequences will be injected into the pronuclei of mouse eggs. The eggs will then be implanted into a pseuodopregnant female. Resulting offspring will be screened for the presence of the appropriate transgene by PCR and Southern hybridization. Mice containing the transgene will be mated to determine whether there is germline transmission, and resulting progeny will be examined for the level and distribution of expression of the POLK or CAT gene to select founder mice that are likely to ubiquitously overexpress the mouse Polk or human POLK gene. Five founder lines for each transgene construct will be established and mated to generate mice homozygous for the Polk transgene. Finally, mice from founder lines containing the pCAG-CAT-Polk transgene will be mated with homozygous mice expressing Cre recombinase to generate strains that widely overexpress the mouse Polk gene. The resulting mice will be assessed for phenotypic abnormalities, including increased levels of spontaneous tumorigenesis. If such overexpression is lethal, the CAG-CAT-Polk transgenic founders can be mated with mice that express Cre-recombinase in an inducible or tissue-specific manner, as described earlier.

D. Studies with Mice Carrying Mutant and Overexpressing Polk Alleles

Polk-defective and Polk-transgenic mice will be maintained on normal dietary regimens and shielded from known environmental carcinogens. The growth, maturation, life span and behavior of the mice will be carefully monitored to determine any spontaneous abnormalities associated with a defective or overexpressed Polk gene. At selected times animals will be sacrificed and complete autopsies performed, including histological examination of multiple organs. Careful attention will be directed to the presence of spontaneous tumors.

Expression of mouse Polk in various tissues at various times during development will be monitored by immunohistochemistry using monoclonal antibodies raised against a specific peptide identified from the mouse Pol κ protein sequence, as well as a human PolκΔN antibody that cross-reacts with mouse Pol κ protein in whole cell extracts. To verify the specificity of any staining observed by immunohistochemistry, in situ hybridization using an antisense riboprobe specific for the mouse Polk cDNA will be performed on the same mouse tissues.

Biopsies will be taken from various parts of the skin and mouse embryonic fibroblast (MEF) cell lines will be established in culture. These cell lines will be quantitatively examined for sensitivity or resistance to killing by a variety of DNA-damaging agents, including UV radiation, 4NQO, γ radiation and hydrogen peroxide, using a dye exclusion assay. In these studies, cells from mice of the identical genetic background with the wild-type Polk allele will be used as normal controls. These cell lines will also be tested for increased or decreased levels of spontaneous mutagenesis using a supF shuttle-vector containing an E. coli tyrosine suppressor tRNA gene as a mutagenic target, as well as sequences permitting replication and selection in bacteria and in mammalian cells (Kraemer and Seidman, 1989). In brief, the supF shuttle-vector will be transfected into different mouse cell lines which are wild type, Polk-deficient or overexpress the Polk gene. After allowing DNA replication in these cells for 2-3 days DNA will be harvested and digested with DpnI to linearize unreplicated DNA. The DNA will then be introduced by electroporation into an indicator strain of E. coli (MBM7070) containing a stop codon in the β-galactosidase gene. If accurate replication of the supF plasmid occurred in the mouse cells the suppressor tRNA will permit expression of β-galactosidase on plates containing X-Gal and blue colonies will be observed. If DNA replication in the mouse cells was error-prone (as might be expected when Polk is overexpressed), mutations that result in partial or total inactivation of supF function will result in colonies that are light blue or white, respectively. The supF gene can then be sequenced to determine the nature of the inactivating mutation. It is hypothesized that cells from the Polk-knockout mice will show decreased mutagenesis compared with wild type, whereas cells from the Polk-overexpressing mice will display increased mutagenesis.

Once the “spontaneous” pathology of Polk-defective mice is clearly established animals will be subjected to thoughtfully designed protocols to determine whether they are unusually resistant to cancers caused by treatment with selected carcinogens, as might be predicted. The carcinogens used will be selected based on our observations from testing the effects of various DNA-damaging agents on human POLK expression, as well as testing the protein's ability to bypass lesions in DNA. In the event that mice defective in Polk are indeed less cancer prone, the potential utility of targeting the human Polκ protein for rational drug design for cancer treatment is of obvious importance. DNA damage-induced mutations that arise during the course of translesion replication are likely to be an important contributory cause in the development of many cancers and error-prone polymerases may thus constitute an attractive target for cancer inhibitors.

### EXAMPLE 7

### Expression of Pol Kappa Protein in the Mouse Adrenal

A mouse mutant with a deletion in exon 6 of the dinB gene was created as described above and confirmed at the nucleic acid and protein level. Analysis of dinB mRNA demonstrated expression of the mutant transcript in mutant mice (FIG. 2). For immunologic confirmation of the mutant, a 14-amino acid peptide of the mouse dinB open reading frame (ORF) with N-terminal cysteine added for conjugation with Keyhole limpit hemocyanin (KLH, Pierce, Rockford, Ill.) was synthesized by the Biopolymer Facility at UTSWMC. The peptide was chosen to have good hydrophilic surface probability and antigenic properties determined by the MacVector 6.5 program. The peptide differed from the human dinB sequence by 10 (underlined) amino acids (CNYLKIDTPRQEANE) (SEQ ID NO:18). Armenian hamsters (Cytogen, Boston, Mass.) were injected intrasplenically with 50 mg peptide-KLH to initiate the immune response. One month later, 50 mg emulsified in complete Freund's adjuvant was injected s.c. Two to 3 booster immunizations with 50 mg in incomplete Fretnd's adjuvant were injected s.c. at 2-week intervals. ELISA and Western blot analysis from intra-orbital blood measured serum antibody titers. Hamsters with high titers were immunized i.v. with 30 mg peptide-KLH 3 days before removing spleens after euthanasia. SP2/O mouse myeloma cells were grown in DMEM containing 2 mM L-glutamine, 100 U penicillin and 100 mg streptomycin/ml with 15% heat-inactivated fetal calf serum (FCS).

A 5:1 mixture of spleen:myeloma cells was centrifuged and fused by the addition of 50% v/v polyethylene glycol 1500 (Boerhinger Mannheim Biochemicals). Cells were distributed into 9 96-well flat-bottom plates in Eagle's medium with hypoxanthine, aminopterin and thymidine (HAT) selection medium (Sigma, St. Louis). ELISA assessed wells with colony growth (415/864) for antibody titer, with 37/415 positive initially. The 37 positive colonies were passed into 24-well plates and a second ELISA detected 29/37 positive titers.

Murine fetal fibroblasts grown on cover glasses were fixed with paraformaldehyde and membranes were rendered permeable by 1% Triton-X 100/PBS. The cells were ‘blocked’ with 5% BSA/PBS, and then incubated with hybridoma supernatant fluids. The cells were stained with goat anti-hamster IgG-FITC (Jackson ImmunoResearch Lab, Inc., Westgove, Pa.). Cells were examined using an UV-fluorescence microscope. Six/29 supernatant fluids were positive in that nuclear membranes fluoresced. Five mAbs (2G10, 7E10. 3B5, 5D8, 6C5) stained in a stippled pattern. Limiting dilutions cloned the hybridomas. (C.B-17 X C57BL/6)F1 severe combined immunodeficiency (SCID) mice were injected with 1 ml pristane i.p., and 7 days later injected with 15 ml rabbit anti-asialo GM1 serum/0.5 mil PBS i.p. to prevent rejection of the hybridomas.

Five-10 million hybridoma cells were injected on the same day as the antiserum. Ascites fluids were collected 2 weeks later and were tested for ELISA titers. The 2G10 clone was tested by immunohistochemistry. Immunostaining was performed at room temperature on a BioTek Solutions TechMate 1000 automated immunostainer (Ventana BioTek Systems, Tucson, Ariz.). Buffers, blocking solutions, streptavidin/biotin complex reagents, chromogen, and hematoxylin counterstain were used as supplied in the Level 2 USA UltraStreptavidin Detection System purchased from Signet Laboratories (Dedham Mass.). Biotinylated secondary antibody purchased from Vector Laboratories (Burlingame, Calif.) Heat induced epitope retrieval (HIER) buffer was obtained from BioPath (Oklahoma City, Okla.). Paraffin sections were cut at 3 microns on a rotary microtome, mounted on positively charged glass slides (POP100 capillary gap slides, Ventana BioTek Systems), and air dried overnight. Positive staining indicative of pot kappa protein was observed in the nuclei of adrenal cortical cells from wild-type mice, whereas adrenal cortical cells from dinB mutant mice were found to be negative for staining.

### EXAMPLE 8

### GST/pol κ Kappa is Able to Bypass a Thymine Glycol Adduct

Primer extension of 5 nM thymine glycol-containing primer-templates was tested using 0.5, 1.0, and 5.0 nM of GST/pol κ kappa (FIG. 3, lanes 2-4). Reactions were performed for 10 min at 37° C. under standard conditions described previously for this enzyme (Gerlach et al., 2001). 1 nM Klenow (exo−) enzyme (FIG. 3, lane 5) and 0.4 U pol delta enzyme (FIG. 3, lanes 6 and 7) were used as controls and are shown to bypass thymine glycol less efficiently. The position of the thymine glycol adduct is indicated at the right and is located at the 30 nucleotide (nt) position. The unextended running start primer (FIG. 3, lane 1) is 20 nucleotides long and the full-length extension product is 53 nucleotides in length.

### EXAMPLE 9

### GST/pol Kappa Preferentially Incorporates A Opposite Thymine Glycol

An oligonucleotide with the sequence 5′ATTCCAGACTGTCAATAACACGGTgGGACCAGTCGATCCTGGGCTGCAGGA ATTC3′ (SEQ ID NO: 19) containing thymine glycol at the position indicated by “Tg”, was annealed to the 5′-³²P-end-labeled primer 5′GAATTCCTGCAGCCCAGGATCGACTGGTCC3′ (SEQ ID NO:20) in a 1:1.5 stoichiometric ratio by heating (10 mM Tris-HCl, 100 mM NaCl, 1 mM Na₂EDTA, 90° C., 5 min) and cooling on the bench top. The annealed primer terminates one base 3′ to the thymine glycol lesion located on the template strand as shown in the scheme at the bottom of the figure. Primer extension reactions (10 mL) were performed using primer/template (5 nM) incubated in 50 mM Tris-HCl (pH 7.0), 5 mM MgCl₂, 1 mM Dithiothreitol, 10 mM NaCl, 1% glycerol, 100 mM total dNTPs, 0.1 mg/mL BSA, 2 nM GST/polK, 10 min, 37° C. unless otherwise indicated. Control lanes 1 and 7 contained an equimolar mix (100 mM total) of each of the four dNTPs but no GST/polK protein. Instead of a mixture of the four dNTPs, lanes 2 and 8 contained only DATP, lanes 3 and 9 only dCTP, lanes 4 and 10 only dGTP, lanes 5 and 11 only dTTP. Lanes 1-6 contained a control primer/template with deoxyguanosine instead of thymidine glycol at the indicated position. Following primer extension, reactions were stopped by addition of 10 mL loading dye (90% formamide, 0.1× TBE, 0.03% xylene cyanole FF), heated to 90° C., 5 min and the volume reduced to 10 mL a speed vac concentrator. 5 mL of the sample was loaded to a 14% denaturing polyacrylamide gel (19:1 acryl: bisacrylamide, 1× TBE, 55° C.) and the gel was run until the xylene cyanole had migrated 28 cm from the origin. Gels were dried under vacuum and exposed to a phosphorimagery screen.

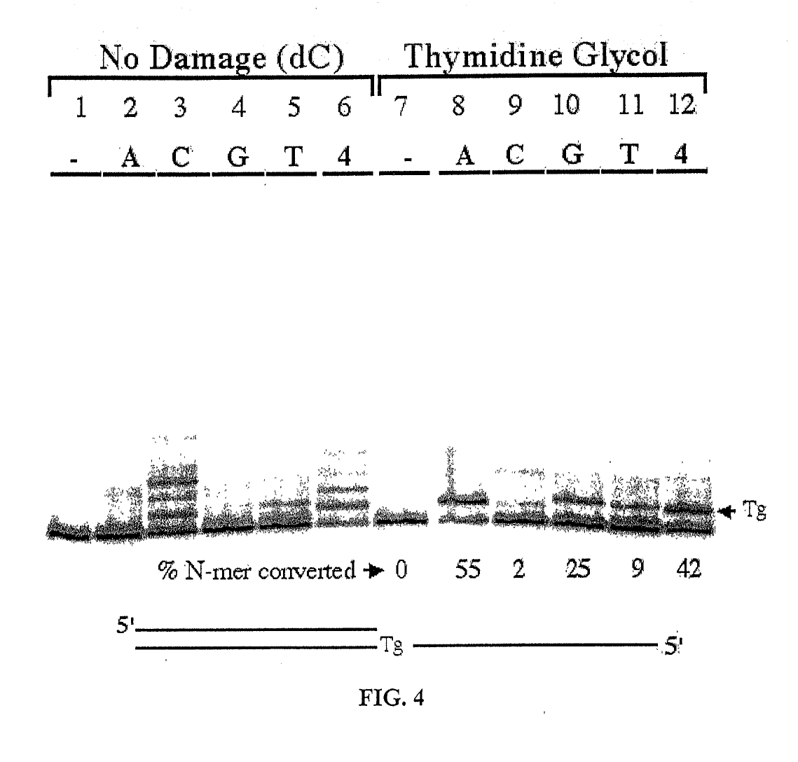

The resulting data was imaged and quantitated on a Typhoon system (Molecular Dynamics) (FIG. 4). Immediately below lanes 7-12 appear background-corrected quantitation of the percentage of ³²P signal in each lane which was extended one or more nucleotides by polK (FIG. 4). The data are consistent with a slight preference by polK for incorporation of deoxyadenosine opposite thymidine glycol. The relative incorporation was A>G>C=T. Hence, it was shown that polK preferentially incorporates the correct nucleotide.

### EXAMPLE 10

### Multiple dinB Transcripts in Mouse Testis

First strand cDNA was synthesized from DNase I-treated mouse testis total RNA (Origen) using the Superscript first-strand synthesis system kit for RT-PCR (Gibco). Expand High Fidelity PCR System (Roche) was used for PCR reactions. PCR primers were 5′AGGCCATGGATAACACAAAGGAAAAGG3′ (SEQ ID NO:21) and 5′ACGGTCGACACGTTGATAAAATGTTCAAAGTTC3′ (SEQ ID NO:22) which flank the open reading frame of the mouse dinB gene. PCR conditions were: 94° C., 15″; 61° C., 30″; 72° C., 2′10″ for 28 cycles. The products were run on a 1% low melting agarose gel (FIG. 5). Following recovery from the gel PCR products were cloned into the pGEM-T easy vector for sequencing. The 2559 bp band represents the full-length dinB ORF. The bands of 2319 bp, 1644 bp and 1404 bp represent transcripts which delete exon7, exon13 and both exons, respectively. These are represented schematically on the right (FIG. 5).

### EXAMPLE 11

### p53-Dependent Induction of dinB Gene Expression in Response to Genotoxic Stress

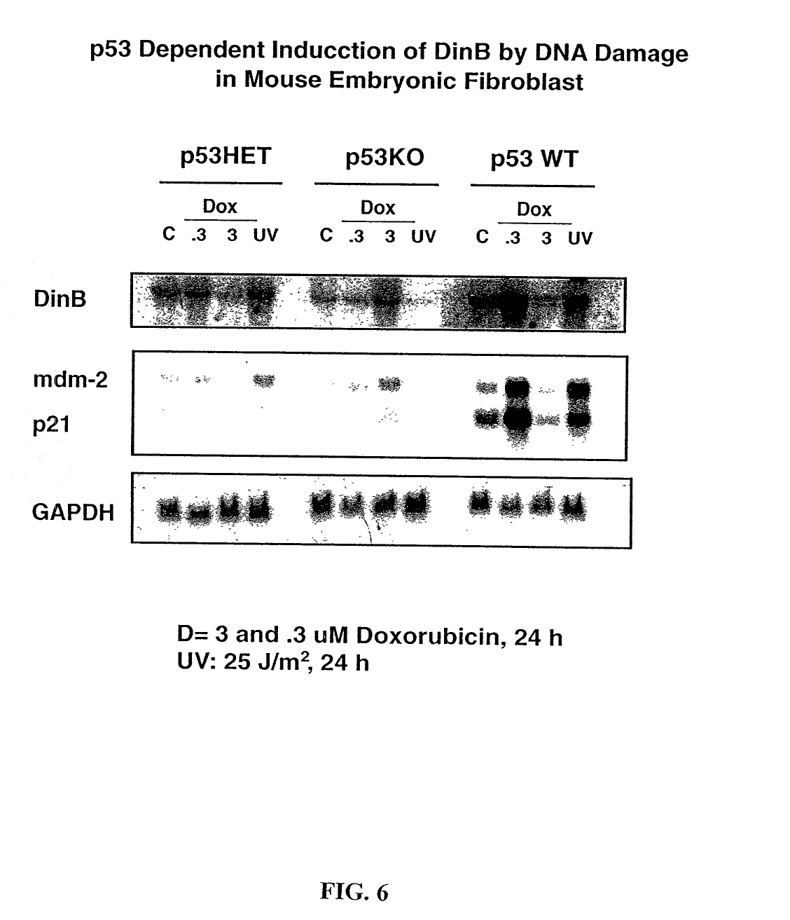

Mouse embryonic fibroblasts (MEFs) derived from p53 mutant (KO), heterozygous (Het) or wild type (WT) embryos (Jacks et al., 1994) were treated with either two different doses of doxorubicin (0.3 μM and 3 μM) or ultraviolet light (UV) (25 J/m²) for 24 hr, or were not treated (C). Cells were harvested by direct addition of a guanidinium thiocyanate solution and total RNA was isolated using cesium chloride gradient centrifugation. Expression of the dinB genes as well as two p53-responsive genes (p21 and MDM-2) was examined by Northern blot analysis as described (Velasco-Miguel et al., 1999). GADPH was used as an RNA loading control. The Northern blot demonstrates enhanced levels of the dinB transcript in wild-type MEFs after exposure to 0.3 μM doxorubicin, and after exposure to UV radiation (FIG. 6). This enhanced expression is p53-dependent.

All of the compositions and methods disclosed and claimed herein can be made and executed without undue experimentation in light of the present disclosure. While the compositions and methods of this invention have been described in terms of embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

EPA No. 320 308

EPA No. 329 822

GB Application No. 2,202,328

GB Application No. 2193095

PCT/US85/01161

PCT/US87/00880

PCT/US89/01025

PCT/US89/05040

U.S. Pat. No. 3,817,837

U.S. Pat. No. 3,850,752

U.S. Pat. No. 3,939,350

U.S. Pat. No. 3,996,345

U.S. Pat. No. 4,162,282

U.S. Pat. No. 4,196,265

U.S. Pat. No. 4,275,149

U.S. Pat. No. 4,277,437

U.S. Pat. No. 4,310,505

U.S. Pat. No. 4,366,241

U.S. Pat. No. 4,533,254

U.S. Pat. No. 4,554,101

U.S. Pat. No. 4,683,195

U.S. Pat. No. 4,683,202

U.S. Pat. No. 4,684,611

U.S. Pat. No. 4,728,575

U.S. Pat. No. 4,728,578

U.S. Pat. No. 4,737,323

U.S. Pat. No. 4,800,159

U.S. Pat. No. 4,879,236

U.S. Pat. No. 4,883,750

U.S. Pat. No. 4,921,706

U.S. Pat. No. 4,946,773

U.S. Pat. No. 4,952,500

U.S. Pat. No. 5,054,297

U.S. Pat. No. 5,279,721

U.S. Pat. No. 5,302,523

U.S. Pat. No. 5,322,783

U.S. Pat. No. 5,354,855

U.S. Pat. No. 5,384,253

U.S. Pat. No. 5,399,363

U.S. Pat. No. 5,464,765

U.S. Pat. No. 5,466,468

U.S. Pat. No. 5,538,877

U.S. Pat. No. 5,538,880

U.S. Pat. No. 5,543,158

U.S. Pat. No. 5,550,318

U.S. Pat. No. 5,563,055

U.S. Pat. No. 5,580,859

U.S. Pat. No. 5,589,466

U.S. Pat. No. 5,591,616

U.S. Pat. No. 5,609,870

U.S. Pat. No. 5,610,042

U.S. Pat. No. 5,641,515

U.S. Pat. No. 5,656,610

U.S. Pat. No. 5,693,762

U.S. Pat. No. 5,702,932

U.S. Pat. No. 5,736,524

U.S. Pat. No. 5,739,169

U.S. Pat. No. 5,780,448

U.S. Pat. No. 5,785,970

U.S. Pat. No. 5,789,215

U.S. Pat. No. 5,824,311

U.S. Pat. No. 5,830,880

U.S. Pat. No. 5,840,873

U.S. Pat. No. 5,843,640

U.S. Pat. No. 5,843,650

U.S. Pat. No. 5,843,651

U.S. Pat. No. 5,843,663

U.S. Pat. No. 5,846,225

U.S. Pat. No. 5,846,233

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,846,945

U.S. Pat. No. 5,849,481

U.S. Pat. No. 5,849,483

U.S. Pat. No. 5,849,486

U.S. Pat. No. 5,849,487

U.S. Pat. No. 5,849,497

U.S. Pat. No. 5,849,546

U.S. Pat. No. 5,849,547

U.S. Pat. No. 5,851,770

U.S. Pat. No. 5,851,772

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,155

U.S. Pat. No. 5,861,244

U.S. Pat. No. 5,863,732

U.S. Pat. No. 5,863,753

U.S. Pat. No. 5,866,331

U.S. Pat. No. 5,866,337

U.S. Pat. No. 5,866,366

U.S. Pat. No. 5,871,986

U.S. Pat. No. 5,879,703

U.S. Pat. No. 5,882,864

U.S. Pat. No. 5,900,481

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,626

U.S. Pat. No. 5,919,630

U.S. Pat. No. 5,922,574

U.S. Pat. No. 5,925,517

U.S. Pat. No. 5,925,525

U.S. Pat. No. 5,925,565

U.S. Pat. No. 5,928,862

U.S. Pat. No. 5,928,869

U.S. Pat. No. 5,928,870

U.S. Pat. No. 5,928,905

U.S. Pat. No. 5,928,906

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,819

U.S. Pat. No. 5,935,825

U.S. Pat. No. 5,939,291

U.S. Pat. No. 5,942,391

U.S. Pat. No. 5,945,100

U.S. Pat. No. 5,980,912

U.S. Pat. No. 5,981,274

U.S. Pat. No. 5,994,624

U.S. Pat. No. 6,020,192

U.S. Pat. No. 6,027,727

WO 84/03564

WO 88/10315

WO 89/06700

WO 90/07641

WO 94/09699

WO 95/06128

WO 99/18933

Abbondanzo et al., Breast Cancer Res. Treat., 16:182(#151), 1990.

Allred et al., Breast Cancer Res. Treat., 16:182(#1 49), 1990.

Almendro et al., J Immunol., 157:5411-5421, 1996.

Altschul et al., Nucleic Acids Res., 25:3389-3402, 1997.

Altschul, S. F., & Koonin, E. V. (1998) Trends Biochem. Sci. 11, 444-447.

Angel et al., Cell, 49:729, 1987b.

Angel et al., Mol. Cell. Biol., 7:2256, 1987a.

Araki et al., Proc. Natl. Acad. Sci. USA 92: 160-164, 1995.

Arap et al., Cancer Res., 55:1351-1354, 1995.

Aravind, L., Walker, D. R., & Koonin, E. V. (1999) Nucleic Acids Res. 27, 1223-1242.

Atchison and Perry, Cell, 46:253, 1986.

Atchison and Perry, Cell, 48:121, 1987.

Austin-Ward, Villaseca, Rev. Med. Chil., 126(7):838-45, 1998.

Ausubel, ed., Current protocols in molecular biology, New York, John Wiley & Sons, 1996.

Baichwal and Sugden, In: Gene Transfer, Kucherlapati R, ed., New York, Plenum Press, pp. 117-148, 1986

Bailly, V., Lauder, S., Prakash, S., & Prakash, L. (1997) J. Biol. Chem. 272, 23360-23365.

Bajorin et al., Proc. Annu. Meet. Am. Soc. Clin. Oncol., 7:A967, 1988.

Baker, G. et al. (eds.), Modern Pharmaceutics, Marcel Dekker, Inc., New York, N.Y., 1990.

Bakhshi et al., Cell, 41(3):899-906, 1985.

Banerji et al., Cell, 27:299, 1981.

Banerji et al., Cell, 35:729, 1983.

Bangham, et al., J Mol. Biol., 13:238-252, 1965.

Bebenek et al., J. Biol. Chem., 270:19516-19523, 1995.

Bebenek et al., Methods Enzyumol, 262:217-232, 1995.

Bell et al., J. Biol. Chem., 272:7345-7351, 1997.

Benjamini, “Immunology: A Short Course,” Wiley-Liss, New York (3rd ed., 1991).

Berkhout et al., Cell, 59:273, 1989.

Berzal-Herranz, A. et al., Genes and Devel., 6:129-134, 1992.

Blanar et al., EMBO J., 8:1139, 1989.

Bodine and Ley, EMBO J., 6:2997, 1987.

Bonavida et al., Int J Oncol, 15:793-802, 1999.

Bonavida et al., Proc Nat'l Acad Sci USA. 97:1754-9, 2000.

Borden K. L., & Freemont, P. S. (1996) Curr. Opin. Struct. Biol. 6, 395-401.

Boshart et al., Cell, 41:521, 1985.

Bosze et al., EMBO J, 5:1615, 1986.

Braddock et al., Cell, 58:269, 1989.

Braithwaite, D. K., & Ito, J. (1993 ) Nucleic Acids Res 21, 787-802.

Brotcorne-Lannoye and Maenhaut-Michel, Proc. Natl. Acad. Sci. USA 83: 3904-3908, 1986.

Brown et al. Breast Cancer Res. Treat., 16:192(#191), 1990.

Brutlag et al., CABIOS, 6:237-245, 1990.

Bukowski et al., Clin. Cancer Res., 4(10):2337-47, 1998.

Bulla and Siddiqui, J. Virol., 62:1437, 1986.

Caldas et al., Nat. Genet., 8:27-32, 1994.

Campbell and Villarreal, Mol. Cell. Biol., 8:1993, 1988.

Campbell, In: Monoclonal Antibody Technology, Laboratory Techniques in Biochemistry and Molecular Biology, Vol. 13, Burden and Von Knippenberg, Eds. pp. 75-83, Amsterdam, Elseview, 1984.

Campere and Tilghman, Genes and Dev., 3:537, 1989.

Campo et al., Nature, 303:77, 1983.

Canfield et al., Methods in Enzymology, 189, 418-422, 1990.

Capaldi et al., Biochem. Biophys. Res. Comm., 76:425, 1977.

Carbonelli et al. FEMS Microbiol Lett. 177(1):75-82, 1999.

Cech et al., Cell, 27:487-496, 1981.

Celander and Haseltine, J. Virology, 61:269, 1987.

Celander et al., J. Virology, 62:1314, 1988.

Chandler et al., Cell, 33:489, 1983.

Chandler et al., Proc Natl Acad Sci USA. 94(8):3596-3601, 1997.

Chang et al., Mol. Cell. Biol., 9:2153, 1989.

Chattejee et al., Proc. Nat'l Acad. Sci. USA., 86:9114, 1989.

Chen and Okayama, Mol. Cell Biol., 7:2745-2752, 1987.

Cheng et al., Cancer Res., 54:5547-5551, 1994.

Cheng, et al., Investigative Radiology, vol. 22, pp. 47-55 (1987).

Choi et al., Cell, 53:519, 1988.

Chou and Fasman, Adv. Enzymol. Relat. Areas Mol. Biol., 47:45-148, 1978a.

Chou and Fasman, Ann. Rev. Biochem., 47:251-276, 1978b.

Chou and Fasman, Biochemistry, 13(2):222-245, 1974a.

Chou and Fasman, Biophys. J, 26:367-384, 1979.

Chowrira, B. H. et al., Biochemistry, 32:1088-1095, 1993.

Chowrira, B. H. et al., J Biol. Chem., 269:25856-25864, 1994.

Christodoulides et al., Microbiology, 144(Pt 11):3027-37, 1998.

Clark et al., J. Mol. Biol. 198, 123-127, 1987.

Cleary and Sklar, Proc. Nat'l. Acad. Sci. USA, 82(21):7439-43, 1985.

Cleary et al., J. Exp. Med., 164(1):315-20, 1986.

Cocea, Biotechniques. 23(5):814-816, 1997.

Cohen et al., J. Cell. Physiol, 5:75, 1987.

Costa et al., Mol. Cell. Biol., 8:81, 1988.

Coupar et al., Gene, 68:1-10, 1988.

Cripe et al., EMBO J., 6:3745, 1987.

Culotta and Hamer, Mol. Cell. Biol., 9:1376, 1989.

Culver et al., Science, 256:1550-1552, 1992.

Cummings and Zoghbi, Hum. Mol. Genet., 9:909-16, 2000.

Dandolo et al., J. Virology, 47:55, 1983.

Davidson et al., J. Immunother., 21(5):389-98, 1998.

De Villiers et al., Nature, 312:242, 1984.

Deamer and P. Uster, Liposomes (M. Ostro, ed.), Marcel Dekker, Inc., New York, pp. 27-52, 1983.

Dejager et al., J. Clin. Invest., 92:894-902, 1993.

Deschamps et al., Science, 230:1174, 1985.

Diaz et al., Int. Immun. 11: 825-833, 1999.

Dillman Cancer Biother. Radiopharm., 14:5-10, 1999.

Doolittle et al., Methods Mol. Biol., 109:215-37, 1999.

Edbrooke et al., Mol. Cell. Biol., 9:1908, 1989.

Edlund et al., Science, 230:912, 1985.

El-Gorab et al., Biochem. Biophys. Acta, 1973, 306, 58-66, 1973.

Esposito et al., Proc. Natl. Acad. Sci. USA 97: 1166-1171, 2000.

Feaver et al., J. Biol. Chem., 2000.

Fechheimer et al., Proc. Nat'l Acad. Sci. USA, 84:8463-8467, 1987.

Felsenstein, Methods Enzymol. 266, 418-427, 1996.

Fendler et al., Catalysis in Micellar and Macromolecular Systems, Academic Press, New York, 1975.

Feng and Holland, Nature, 334:6178, 1988.

Fetrow and Bryant, Biotech., 11:479-483, 1993.

Firak and Subramanian, Mol. Cell. Biol., 6:3667, 1986.

Fodor et al., Science, 251:767-773, 1991.

Foecking and Hofstetter, Gene, 45(l):101-5, 1986.

Forster and Symons, Cell, 49:211-220, 1987.

Fraley et al., Proc. Nat'l Acad. Sci. USA, 76:3348-3352, 1979.

Friedberg & Gerlach, Cell, 98:413-6, 1999.

Friedberg & Gerlach, Cell, in press, 1999.

Friedberg et al., DNA Repair and Mutagenesis (Am. Soc. Microbiol., Washington, D.C.), 1995.

Friedmann, Science, 244:1275-1281, 1989.

Frohman, In: PCR Protocols. A Guide To Methods And Applications, Academic Press, N.Y., 1990.

Fujita et al., Cell, 49:357, 1987.

Gabizon et al., Cancer Res., 50(19):6371-8, 1990.

Gerlach et al., Proc. Natl. Acad. Sci. USA 96: 11922-11927, 1999.

Gerlach et al., Nature (London), 328:802-805, 1987.

Gerlach et al., 2001 J. Biol. Chem., Vol. 276, Issue 1, 92-98, Jan. 5, 2001

Ghose and Blair, Crit. Rev. Ther. Drug Carrier Syst., 3(4):263-359, 1987.

Ghosh and Bachhawat, In: Liver diseases, targeted diagnosis and therapy using specific receptors and ligands, (Wu G, Wu C ed.), New York: Marcel Dekker, pp. 87-104, 1991.

Gilles et al., Cell, 33:717, 1983.

Gliniak et al., Cancer Res. 59:6153-8, 1999.

Gloss et al., EMBO J., 6:3735, 1987.

Godbout et al., Mol. Cell. Biol., 8:1169, 1988.

Goodboum and Maniatis, Proc. Nat'l Acad. Sci. USA, 85:1447, 1988.

Goodbourn et al., Cell, 45:601, 1986.

Gopal, Mol. Cell Biol., 5:1188-1190, 1985.

Graham and Van Der Eb, Virology, 52:456-467, 1973.

Greene et al., Immunology Today, 10:272, 1989.

Gregoriadis, ed., Drug Carriers In Biology And Medicine, pp. 287-341, 1979.

Gregoriadis, G., ed., Liposome Technology, vol. I, pp. 30-35, 51-65 and 79-107 (CRC Press Inc., Boca Raton, Fla., 1984.

Grosschedl and Baltimore, Cell, 41:885, 1985.

Gu et al., Science 265: 103-106, 1994.

Gulbis et al., Hum. Pathol., 24:1271-85, 1993.

Hacia et al., Nature Genetics, 14:441-447, 1996.

Hanibuchi et al., Int. J. Cancer, 78(4):480-5, 1998.

Harland and Weintraub, J. Cell Biol, 101:1094-1099, 1985.

Harlow and Lane, “Antibodies: A Laboratory Manual,” Cold Spring Harbor Laboratory, 1988.

Haseloff and Gerlach, Nature, 334:585-591, 1988.

Haslinger and Karin, Proc. Nat'l Acad. Sci. USA., 82:8572, 1985.

Hauber and Cullen, J. Virology, 62:673, 1988.

Hellstrand et al., Acta. Oncol., 37(4):347-53, 1998.

Hen et al., Nature, 321:249, 1986.

Hensel et al., Lymphokine Res., 8:347, 1989.

Hernonat and Muzyczka, Proc. Nat'l. Acad. Sci. USA, 81:6466-6470, 1984.

Herr and Clarke, Cell, 45:461, 1986.

Hindges, and Hübscher, Biol. Chem. 378, 345-362, 1997.

Hirochika et al., J. Virol., 61:2599, 1987.

Hirsch et al., Mol. Cell. Biol., 10:1959, 1990.

Holbrooketal., Virology, 157:211, 1987.

Hollstein et al., Science 253:49-53, 1991.

Hope et al., Biochimica et Biophysica Acta, 812: 55-65, 1985.

Horlick and Benfield, Mol. Cell. Biol., 9:2396, 1989.

Horwich et al. J. Virol., 64:642-650, 1990.

Huang et al., Cell, 27:245, 1981.

Hug et al., Mol Cell Biol., 8:3065, 1988.

Hui and Hashimoto, Infect. Immun., 66(11):5329-36, 1998.

Hussussian et al., Nature Genetics, 15-21, 1994.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol., 7:2558, 1987.

Imperiale and Nevins, Mol. Cell. Biol., 4:875, 1984.

Inouye et al., Nucleic Acids Res., 13:3101-3109 1985.

Irie & Morton, Proc. Nat'l Acad. Sci. USA 83:8694-8698, 1986

Irie et al., “Melanoma gangliosides and human monoclonal antibody,” In: Human Tumor Antigens and Specific Tumor Therapy, Metzgar & Mitchell (eds.), Alan R. Liss, Inc., New York, pp. 115-126, 1989.

Jacobs et al., J. Exp. Med. 187: 1735-1743, 1998.

Jacks et al. Current Biol. 4, 1-7,1994

Jakobovits et al., Mol. Cell. Biol., 8:2555, 1988.

Jameel and Siddiqui, Mol. Cell. Biol., 6:710, 1986.

Jameson and Wolf, Comput. Appl. Biosci., 4(1):181-186, 1988.

Jaynes et al., Mol. Cell. Biol., 8:62, 1988.

Johnson et al., J. Biol. Chem. 274: 15975-15977, 1999c.

Johnson et al., J. Biol. Chem. 275:7447-7450, 2000b.

Johnson et al., J. Virol., 67:438-445,1993.

Johnson et al., Mol. Cell. Biol., 9:3393, 1989.

Johnson et al., Proc. Natl. Acad. Sci. USA 97: 3838-3843, 2000.

Johnson et al., Science 283, 1001-1004, 1999a.

Johnson et al., Science 285, 263-265, 1999b.

Jones et al., Nucleic Acids Res. 25, 7119-7131, 1988.

Joyce, Nature, 338:217-244, 1989.

Ju et al., Gene Ther., 7(4):329-38, 2000.

Kadesch and Berg, Mol. Cell. Biol., 6:2593, 1986.

Kaeppler et al., Plant Cell Reports 9: 415-418, 1990.

Kamb et al., Nature Genetics, 8:22-26, 1994.

Kamb et al., Science, 2674:436-440, 1994.

Kaneda et al., Science, 243:375-378, 1989.

Karin et al., Mol. Cell. Biol., 7:606, 1987.

Katinka et al., Cell, 20:393, 1980.

Katinka et al., Nature, 290:720, 1981.

Kato et al., J. Biol. Chem., 266:3361-3364, 1991.

Kawamoto et al., Mol. Cell. Biol., 8:267, 1988.

Keane et al., Cancer Res. 59:734-41, 1999.

Kerr et al., Br. J. Cancer, 26(4):239-57, 1972.

Kiledjian et al., Mol. Cell. Biol., 8:145, 1988.

Kim and Cech, Proc. Nat'l Acad. Sci. USA, 84:8788-8792, 1987.

Kim et al., Proc. Natl. Acad. Sci. USA 94: 13792-13797, 1997.

Klamut et al., Mol. Cell. Biol., 10:193, 1990.

Koch et al., Mol. Cell. Biol., 9:303, 1989.

Kozak, J. Cell Biol. 108, 229-241, 1989.

Kraemer et al., Mutat. Res. 220: 61-72, 1989.

Kraus et al. FEBS Lett., 428(3):165-170, 1998.

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984a.

Kriegler et al., Cell, 53:45, 1988.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Kuhl et al., Cell, 50:1057, 1987.

Kulaeva et al., Mut. Res. 357, 245-253, 1996.

Kunkel & Bebenek, Annu. Rev. Biochem., 69, in press, 2000.

Kunkel, J. Biol. Chem., 261:13581-13587, 1986.

Kunz et al., Nucl. Acids Res., 17:1121, 1989.

Kwoh et al., Proc. Nat. Acad. Sci. USA, 86: 1173, 1989.

Kyte and Doolittle, J. Mol. Biol., 157(1):105-132, 1982.

Landis et al., CA Cancer J Clin., 48:6, 1998.

Lareyre et al., J. Biol Chem., 274(12):8282-8290, 1999.

Larimer et al., J. Bacteriol. 171, 230-237, 1989.

Larsen et al., Proc. Nat'l Acad. Sci. USA., 83:8283, 1986.

Laspia et al., Cell, 59:283, 1989.

Latimer et al., Mol. Cell. Biol., 10:760, 1990.

Lee et al., J Auton Nerv Syst. 74(2-3):86-90, 1997.

Lee et al., Nature, 294:228, 1981.

Lee et al., Nucleic Acids Res., 12:4191-206, 1984.

Levenson et al., Hum Gene Ther. 20;9(8):1233-1236, 1998.

Levine, The Molecular Basis of Cancer, Mendelsohn, et al., eds. WB Saunders Co., Philadelphia, 1995.

Levinson et al., Nature, 295:79, 1982.

Li & Herskowitz, Science 262, 1870-1874, 1993

Lieber and Strauss, Mol. Cell. Biol., 15: 540-551, 1995.

Lin et al., Mol. Cell. Biol., 10:850, 1990.

Luria et al., EMBO J., 6:3307, 1987.

Lusky and Botchan, Proc. Nat'l Acad. Sci. USA., 83:3609, 1986.

Lusky et al., Mol. Cell. Biol., 3:1108, 1983.

Macejak and Sarnow, Nature, 353:90-94, 1991.

Majors and Varnus, Proc. Nat'l Acad. Sci. USA., 80:5866, 1983.

Marsters et al., Recent Prog Horm Res 54:225-34, 1999.

Martin et al., Nature, 345(6277):739-743, 1990.

Masutani et al., EMBO J. 18, 3491-3501, 1999.

Masutani et al., EMBO J. 18: 3491-3501, 1999a.

Masutani et al., Nature 399: 700-704, 1999b.

Matsuda et al., Nature, 404:1011-1013, 2000.

Mayer et al., Biochimica et Biophysica Acta, vol. 858, pp. 161-168, 1986.

Mayhew et al., Biochimica et Biophysica Acta, vol. 775, pp. 169-174, 1984.

Mayhew et al., Methods in Enzymology, vol. 149, pp. 64-77, 1987.

McConnell et al., Biochemistry 35, 8268-8274, 1996.

McDonald et al., Genetics 147: 1557-1568, 1997.

McDonald et al., Genomics 60: 20-30, 1999.

McDonald et al., Nat. Genet. 15, 417-474, 1997

McNeall et al., Gene, 76:81, 1989.

Michel and Westhof, J. Mol. Biol., 216:585-610, 1990.

Miksicek et al., Cell, 46:203, 1986.

Mitchell et al., Ann. N. Y Acad. Sci., 690:153-166, 1993.

Mitchell et al., J. Clin. Oncol,. 8(5):856-859, 1990.

Mordacq and Linzer, Genes and Dev., 3:760, 1989.

Moreau et al., Nucl. Acids Res., 9:6047, 1981.

Mori et al., Cancer Res., 54:3396-3397, 1994.

Morton and Ravindranath, M. H. Current concepts concerning melanoma vaccines. In Tumor Immunology, Dalgleish A G (ed.), London: Cambridge University Press, 1-55, 1996.

Morton et al., Ann. Surg. 216: 463-482, 1992.

Muesing et al., Cell, 48:691, 1987.

Nakamura et al., In: Enzyme Immunoassays: Heterogeneous and Homogeneous Systems, Chapter 27, 1987.

Nelson et al., Nature 382, 729-731, 1996.

Ng et al., Nuc. Acids Res., 17:601, 1989.

Nicolas and Rubenstein, In. Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau et al., Methods EnzymoL, 149:157-176, 1987.

Nobri et al., Nature, 368:753-756, 1995.

Nomoto et al., Gene, 236(2):259-271, 1999.

Ogi et al., Genes Cells, 4:607-618, 2000.

Ohara et al., Proc. Nat'l Acad. Sci. USA, 86: 5673-5677, 1989.

Ohashi et al., Genes Dev., in press, 2000.

Ohashi et al., Genes Dev., in press, 2000b.

Ohmori et al., Mut. Res. 347: 1-7, 1995.

Okamoto et al., Proc. Nat'l Acad. Sci. USA, 91:11045-11049, 1994.

Omirulleh et al., Plant Mol. Biol., 21:415-28, 1993.

Ondek et al., EMBO J., 6:1017, 1987.

Orlow et al., Cancer Res., 54:2848-2851, 1994.

Omitz et al., Mol. Cell. Biol., 7:3466, 1987.

Osheroff et al., J. Biol. Chem., 274:20749-20752, 1999.

Osheroff et al., J. Biol. Chem., 274:3642-3650, 1999.

Palmiter et al., Nature, 300:611, 1982.

Palukaitis et al., Virology, 99:145-151, 1979.

Pease et al., Proc. Natl. Acad. Sci. USA, 91:5022-5026, 1994.

Pech et al., Mol. Cell. Biol., 9:396, 1989.

Pelletier and Sonenberg, Nature, 334:320-325, 1988.

Perez-Stable and Constantini, Mol. Cell. Biol., 10:1116, 1990.

Perriman. et al., Gene, 113:157-163, 1992.

Perrotta and Been, Biochemistry 31:16, 1992.

Picard and Schaffiner, Nature, 307:83, 1984.

Pietras et al., Oncogene, 17(17):2235-49, 1998.

Pinkert et al., Genes and Dev., 1:268, 1987.

Poch et al., EMBO J. 8, 3867-3874, 1989.

Ponta et al., Proc. Nat'l Acad. Sci. USA., 82:1020, 1985.

Porton et al., Mol. Cell Biol., 10:1076, 1990.

Potrykus et al., Mol. Gen. Genet., 199:183-188, 1985.

Prelich et al., Nature 326, 517-520, 1987.

Prody, G. A. et al., Science, 231, 1577-1580, 1986.

Qin et al., Proc. Nat'l Acad. Sci. USA, 95(24):1411-6, 1998.

Queen and Baltimore, Cell, 35:741, 1983.

Quinn et al., Mol. Cell. Biol., 9:4713, 1989.

Radman, Nature 401: 866-869, 1999.

Rajewsky et al., J. Clin. Invest. 98: 600-603, 1996.

Rajewsky, Nature 381: 751-758, 1996.

Ramírez-Solis et al., Methods Enzymol. 225: 855-879m 1993.

Ravindranath and Morton, Intern. Rev. Immunol. 7: 303-329, 1991.

Redondo et al., Science, 247:1225, 1990.

Reinhold-Hurek and Shub, Nature, 357:173-176, 1992.

Reisman and Rotter, Mol. Cell. Biol., 9:3571, 1989.

Remington's Pharmaceutical Sciences, 15^{th} ed., pages 1035-1038 and 1570-1580, Mack Publishing Company, Easton, Pa., 1980.

Resendez Jr. et al., Mol. Cell. Biol., 8:4579, 1988.

Reuven et al., J. Biol. Chem. 274: 31763-31766, 1999.

Reuven et al., Mol. Cell 2: 191-199, 1998.

Richter et al., Mol. Gen. Genet. 231, 194-200, 1992.

Ridgeway, In: Vectors: A survey of molecular cloning vectors and their uses, Rodriguez R L, Denhardt D T, ed., Stoneham:Butterworth, pp. 467-492, 1988.

Ripe et al., Mol. Cell. Biol., 9:2224, 1989.

Rippe et al., Mol. Cell Biol., 10:689-695, 1990.

Rittling et al., Nucl. Acids Res., 17:1619, 1989.

Roest et al., Cell, 86:799-810, 1996.

Rosen et al., Cell, 41:813, 1988.

Rosenberg et al., Ann. Surg., 210:474, 1989.

Rosenberg et al., N. Engl. J. Med., 319:1676, 1988.

Roush et al., Mol. Gen. Gen. 257, 686-692, 1998.

Sachs, Cell 74, 413-421, 1993.

Saitou et al., Mol. Biol. Evol. 4, 406-425, 1987.

Sakai and Miyazaki Biochem. Biophys. Res. Commun. 237: 318-324, 1997.

Sakai et al., Genes and Dev., 2:1144, 1988.

Sambrook et al., In: Molecular Cloning: A Laboratory Manual, 2nd Ed., Cold Spring Harbor Press, Cold Spring Harbor, N.Y., 1989.

Sarver, et al., Science, 247:1222-1225, 1990.

Satake et al., J. Virology, 62:970, 1988.

Satumo et al., J. Mol. Biol. 283, 633-642, 1998.

Scanlonet al., Proc. Nat'l Acad. Sci. USA, 88:10591-10595, 1991.

Schaffner et al., J. Mol. Biol., 201:81, 1988.

Searle et al., Mol. Cell. Biol., 5:1480, 1985.

Serrano et al., Nature, 366:704-707, 1993.

Serrano et al., Science, 267:249-252, 1995.

Sharp and Marciniak, Cell, 59:229, 1989.

Shaul and Ben-Levy, EMBO J.,6:1913, 1987.

Sherman et al., Mol. Cell. Biol., 9:50, 1989.

Shinoda, K. et al., Colloidal Surfactant, Academic Press, especially “The Formation of Micelles”, Ch. 1, 1-96, 1963.

Shoemaker et al., Nature Genetics, 14:450-456, 1996.

Sioud et al., J. Mol. Biol., 223:831-835, 1992.

Sleigh and Lockett, J. EMBO, 4:3831, 1985.

Spalholz et al., Cell, 42:183, 1985.

Spandau and Lee, J. Virology, 62:427, 1988.

Spandidos and Wilkie, EMBO J., 2:1193, 1983.

Stephens and Hentschel, Biochem. J, 248:1, 1987.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Symons, Ann. Rev. Biochem., 61:641-671, 1992.

Symons, Nucl. Acids Res., 9:6527-6537, 1981.

Szoka et al., Proc. Natl. Acad. Sci., 75:4194-4198, 1978.

Takebe et al., Mol. Cell. Biol., 8:466, 1988.

Tang et al., Proc. Natl. Acad. Sci. USA 95: 9755-9760, 1998.

Tang et al., Nature 404: 1014-1018, 2000.

Tang et al., Proc. Natl. Acad. Sci. USA 96: 8919-8924, 1999.

Tavernier et al., Nature, 301:634, 1983.

Taylor and Kingston, Mol. Cell. Biol., 10: 165, 1990a.

Taylor and Kingston, Mol. Cell. Biol., 10:176, 1990b.

Taylor et al., J. Biol. Chem., 264:15160, 1989.

Temin, In. Gene Transfer, Kucherlapati (ed.), New York: Plenum Press, pp. 149-188, 1986.

Templeton et al., Nat. Biotechnol., 15(7):647-52, 1997.

Thiesen et al., J. Virology, 62:614, 1988.

Thomas et al., Biochemistry, 30:11751-11759, 1991.

Thompson et al. Nature Medicine, 1:277-278, 1995.

Thompson, et al., Nucleic Acids Res. 22, 4673-4680, 1994.

Tonk et al., Amer. Jour. Med. Genet. 61, 16-20, 1996.

Treisman, Cell, 42:889, 1985.

Tronche et al., Mol. Biol. Med., 7:173, 1990.

Tronche et al., Mol. Cell. Biol., 9:4759, 1989.

Trudel and Constantini, Genes and Dev., 6:954, 1987.

Tsujimoto and Croce, Proc. Natl. Acad. Sci. USA, 83(14):5214-8, 1986.

Tsujimoto et al., Science, 228(4706):1440-3, 1985.

Tsumaki et al., J Biol Chem. 273(36):22861-22864, 1998.

Tyndall et al., Nuc. Acids. Res., 9:6231, 1981.

Vannice and Levinson, J. Virology, 62:1305, 1988.

Vasseur et al., Proc. Nat'l Acad. Sci. USA., 77:1068, 1980.

Velasco-Miguel et al. Oncogene, 18, 127-137, 1999

Wada et al., Nucleic Acids Res., 18:2367-2411, 1990.

Wagner et al., Mol. Cell 40: 281-286, 1999.

Walker et al., Proc. Nat'l Acad. Sci. USA, 89:392-396 1992.

Wang and Calame, Cell, 47:241, 1986.

Wang et al., In Eukaryotic DNA Replication. A Practical Approach (ed. S. Cotterill), pp. 67-92. Oxford University Press, NY, 1999.

Wang et al., Science, 289:774-9, 2000.

Wawrzynczak & Thorpe, Cancer Treat. Res., 37:239-51, 1988.

Weber et al., Cell, 36:983, 1984.

Weinberg, Science, 254:1138-1145, 1991.

Weinberger et al. Mol. Cell. Biol., 8:988, 1984.

Winoto and Baltimore, Cell, 59:649, 1989.

Wolf et al., Comput. Appl. Biosci., 4(l):187-191, 1988.

Wong et al., Gene, 10:87-94, 1980.

Woodgate & Sedgwick, Mol. Microbiol. 6, 2213-2218., 1992

Wu et al., Biochem. Biophys. Res. Commun., 233(1):221-6, 1997.

Yuan and Altman, Science, 263:1269-1273, 1994.

Yuan et al., Proc. Nat'l Acad. Sci. USA, 89:8006-8010, 1992.

Yutzey et al. Mol. Cell. Biol., 9:1397, 1989.

Zaychikov et al., Science 273, 107-109, 1996.

Zhao-Emonet et al., Biochem. Biophys. Acta., 1442(2-3):109-19, 1998.

What is claimed is:

1. An isolated and purified polynucleotide comprising a nucleic acid sequence encoding a mammalian pol κ polypeptide.

2. The polynucleotide of claim 1, wherein the polypeptide is a murine polypeptide.

3. The polynucleotide of claim 1, wherein the polypeptide is a human polypeptide.

4. The polynucleotide of claim 1, comprising a nucleic acid sequence encoding at least 10 contiguous amino acid residues of SEQ ID NO:2 or SEQ ID NO:4.

5. The polynucleotide of claim 1, comprising a nucleic acid sequence encoding at least 20 contiguous amino acid residues of SEQ ID NO:2 or SEQ ID NO:4.

6. The polynucleotide of claim 1, comprising a nucleic acid sequence encoding at least 40 contiguous amino acid residues of SEQ ID NO:2 or SEQ ID NO:4.

7. The polynucleotide of claim 1, comprising a nucleic acid sequence encoding at least 60 contiguous amino acid residues of SEQ ID NO:2 or SEQ ID NO:4.

8. The polynucleotide of claim 1, comprising a nucleic acid sequence encoding at least 100 contiguous amino acid residues of SEQ ID NO:2 or SEQ ID NO:4.

9. The polynucleotide of claim 1, comprising a nucleic acid sequence encoding SEQ ID NO:2 or SEQ ID NO:4.

10. The polynucleotide of claim 1, comprising at least 20 contiguous bases of SEQ ID NO:1 or SEQ ID NO:3.

11. The polynucleotide of claim 1, comprising at least 30 contiguous bases of SEQ ID NO:1 or SEQ ID NO:3.

12. The polynucleotide of claim 1, comprising at least 50 contiguous bases of SEQ ID NO:1 or SEQ ID NO:3.

13. The polynucleotide of claim 1, comprising at least 80 contiguous bases of SEQ ID NO:1 or SEQ ID NO:3.

14. The polynucleotide of claim 1, comprising at least 100 contiguous bases of SEQ ID NO:1 or SEQ ID NO:3.

15. The polynucleotide of claim 1, comprising SEQ ID NO:1 or SEQ ID NO:3.

16. An isolated and purified polynucleotide encoding at least 20 contiguous nucleotides of SEQ ID NO: 1.

17. The polynucleotide of claim 16, comprising at least 30 contiguous bases of SEQ ID NO:1.

18. The polynucleotide of claim 17, comprising at least 50 contiguous bases of SEQ ID NO:1.

19. The polynucleotide of claim 18, comprising at least 80 contiguous bases of SEQ ID NO:1.

20. The polynucleotide of claim 19, comprising at least 100 contiguous bases of SEQ ID NO:1.

21. The polynucleotide of claim 16, comprising a nucleic acid sequence encoding at least 20 contiguous amino acid residues of SEQ ID NO:2.

22. The polynucleotide of claim 16, comprising a nucleic acid sequence encoding at least 40 contiguous amino acid residues of SEQ ID NO:2.

23. The polynucleotide of claim 16, comprising a nucleic acid sequence encoding at least 60 contiguous amino acid residues of SEQ ID NO:2.

24. The polynucleotide of claim 16, comprising a nucleic acid sequence encoding at least 100 contiguous amino acid residues of SEQ ID NO:2.

25. The polynucleotide of claim 16, comprising a nucleic acid sequence encoding SEQ ID NO:2.

26. An isolated and purified mammalian pol κ polypeptide comprising at least 10 contiguous amino acids of SEQ ID NO:2 or SEQ ID NO:4.

27. The polypeptide of claim 26, comprising at least 20 contiguous amino acids of SEQ ID NO:2 or SEQ ID NO:4.

28. The polypeptide of claim 27, comprising at least 30 contiguous amino acids of SEQ ID NO:2 or SEQ ID NO:4.

29. The polypeptide of claim 28, comprising at least 40 contiguous amino acids of SEQ ID NO:2 or SEQ ID NO:4.

30. The polypeptide of claim 29, comprising at least 75 contiguous amino acids of SEQ ID NO:2 or SEQ ID NO:4.

31. The polypeptide of claim 30, comprising at least 100 contiguous amino acids of SEQ ID NO:2 or SEQ ID NO:4.

32. The polypeptide of claim 31, comprising at least SEQ ID NO:2 or SEQ ID NO:4.

33. An expression vector comprising a nucleic acid sequence encoding a mammalian polκ polypeptide.

34. The polynucleotide of claim 33, wherein the polypeptide is a murine polypeptide.

35. The polynucleotide of claim 33, wherein the polypeptide is a human polypeptide.

36. The expression vector of claim 33, wherein the nucleic acid sequence comprises at least 20 contiguous bases of SEQ ID NO:1.

37. The expression vector of claim 36, wherein the nucleic acid sequence comprises at least 50 contiguous bases of SEQ ID NO:1.

38. The expression vector of claim 37, wherein the nucleic acid sequence comprises at least 100 contiguous bases of SEQ ID NO:1.

39. The expression vector of claim 33, wherein the nucleic acid sequence encodes at least 10 contiguous amino acids of SEQ ID NO:2.

40. The expression vector of claim 33, wherein the nucleic acid sequence encodes at least 40 contiguous amino acids of SEQ ID NO:2.

41. The expression vector of claim 33, wherein the nucleic acid sequence encodes at least 100 contiguous amino acids of SEQ ID NO:2.

42. The expression vector of claim 33, wherein the nucleic acid sequence encodes SEQ ID NO:2.

43. The expression vector of claim 42, wherein the nucleic acid sequence comprises a promoter operably linked to the pol κ-encoding nucleic acid sequence.

44. The expression vector of claim 42, wherein the expression vector is a viral vector.

45. A method of preparing recombinant pol κ comprising:
(a) transfecting a cell with a polynucleotide comprising a nucleic acid sequence encoding a polκ polypeptide to produce a transformed host cell; and
(b) maintaining the transformed host cell under biological conditions sufficient for expression of the pol κ polypeptide in the host cell.

46. The method of claim 45, wherein the nucleic acid sequence encodes at least 100 contiguous amino acids of SEQ ID NO:2.

47. The method of claim 45, wherein the nucleic acid sequence encodes SEQ ID NO:2.

48. A method of treating a pre-cancer or cancer cell comprising providing to the cell an effective amount of a pol κ modulator, wherein the modulator reduces polκ activity in the cell.

49. The method of claim 48, wherein the modulator reduces polκ activity by reducing DNA binding or polymerization of a nucleic acid molecule.

50. The method of claim 48, wherein the modulator decreases the amount of pol κ in the cell.

51. The method of claim 48, wherein the modulator decreases expression of pol κ.

52. The method of claim 48, wherein the modulator decreases transcription of pol κ.

53. The method of claim 48, wherein the modulator decreases translation of pol κ.

54. The method of claim 48, wherein the modulator specifically binds pol κ.

55. The method of claim 54, wherein the modulator is an antibody.

56. The method of claim 48, wherein the modulator is provided to the cell by an expression cassette comprising a nucleic acid segment encoding the modulator.

57. The method of claim 48, wherein the modulator of pol κ is a nucleic acid containing a promoter operably linked to a nucleic acid segment encoding at least 30 contiguous nucleotides of SEQ ID NO:1 or SEQ ID NO:3. [SEQ ID NO:1 will be human cDNA sequence; SEQ ID NO:3 will be mouse cDNA sequence].

58. The method of claim 57, wherein the nucleic acid segment is positioned, in reverse orientation, under the control of a promoter that directs expression of an antisense product.

59. The method of claim 48, wherein the cell is in an animal.

60. A method of treating a patient with cancer comprising administering to the a subject a pol κ modulator and a second anti-cancer treatment.

61. The method of claim 60, wherein the second anti-cancer treatment is surgery, gene therapy, chemotherapy, radiotherapy, or immunotherapy.

62. A method of treating a pre-cancer or cancer cell comprising contacting the cell with an effective amount of an expression vector comprising a polynucleotide encoding a polκ polypeptide under the transcriptional control of a promoter, wherein the cancer cell is conferred a therapeutic benefit.

63. A method of reducing DNA mutagenesis in a cell comprising administering a polκ modulator in an amount effective to reduce DNA mutagenesis in the cell.

64. A method of increasing DNA mutagenesis in a cell comprising providing to the cell an expression vector comprising a polynucleotide encoding a pol κ polypeptide under the transcriptional control of a promoter, wherein expression of the pol κ polypeptide is at a level effective to increase mutagenesis in the cell.

65. The method of claim 64, wherein the polκ polypeptide comprises at least 20 contiguous amino acids from SEQ ID NO:2.

66. The method of claim 64, wherein the polynucleotide comprises at least 40 contiguous nucleic acids from SEQ ID NO:1.

67. A method of treating a patient with pre-cancer or cancer comprising administering to the patient an amount of a polκ modulator effective to reduce polκ activity, thereby conferring a therapeutic benefit on the subject.

68. A method of identifying a modulator of a polκ polypeptide comprising:
(a) contacting the polκ polypeptide with a candidate substance; and
(b) assaying whether the candidate substance modulates the polκ polypeptide.

69. The method of claim 68, wherein the assaying compares the activity of the polκ polypeptide in the presence and absence of the candidate substance.

70. The method of claim 68, wherein the assaying is done by determining whether the candidate substance specifically interacts with the polκ polypeptide.

71. A method of diagnosing cancer in a subject comprising:
(a) obtaining a sample from the subject;
(b) evaluating pol κ in the sample.

72. The method of claim 71, wherein evaluating pol κ comprises assaying the level of pol κ activity.

73. The method of claim 71, wherein evaluating pol κ comprises assaying the amount of pol κ polypeptide.

74. The method of claim 73, wherein the assaying employs an antibody that specifically binds pol κ.

75. The method of claim 71, wherein evaluating pol κ comprises evaluating a genomic DNA sequence encoding pol κ.

76. A method of treating a trinucleotide repeat disease in a subject comprising administering to the subject an effective amount of an expression vector comprising a polynucleotide encoding a pol κ polypeptide under the transcriptional control of a promoter, wherein a pol κ polypeptide is expressed in the subject.
